SEQUENCE LISTING (459 sequences)

SEQ ID NO: 1 - DNA, 1525 bp, Triticum aestivum
  source 1..1525: mol_type = genomic DNA
atggggcggccgccgtgctgcgacaaggccaacgtgaagaaggggccgtggacggcggaggaggacgccaagctgctggcctacacctccaaccatggcaccggcaactggacctctgttccccagagggcaggtcggaaccctctcccccggccggccggatcggcgtctggcatcgaaatggtgttggttttgcatggtttctggctgatgtgtgttgcgtcatggatgggtgcgtgctgcagggctgaagcggtgcgggaagagctgcaggctgaggtacaccaactacctgaggcccaacctcaagcacgagaacttcacgcaggaggaggaggagctcatcgtcaccctccatgccatgctgggaagcaggtgcatctctgtacatgcatgtcatccatcggttactttctcgattttgtcatcaagttttcggtgaatgcgaaactttgctatggacagactactgtttctgtcatcattcctgctataaactttgcatccatatactctagcgtacgttcagatcttatagatttatggaagttctaaaaacctcacgttggtgtatcattcagacaattgctcatacaccggaaatcacgtcctacttctattcaagaatttactttagccgttcattcataaagtagaaacaaatgtagtttaatccagtaaccagtctcgtgtctacggttcttctgaatatattctgtttcctggtagaatgtagtgaaaagctgaatattgggtgaagaacagatctgaatagctgtcttaaattgatagtgtttgacagaaaatactccacactgagcgtaatataacatgaaagtacgtgtacgtgttgggtgcatgcaggtggtctctgatcgcgaaccagctgccggggcggacggacaacgacgtcaagaattactggaacaccaagctgagcaagaagctgcggcagcggggcatcgaccccatcacccatcgccccatcgccgacctcatgcagagcatcggcaccctccccatccgcccgccgcccagcgccgcgggtgcctcctcgtcctcctacatccccgtgaacccagcggcggcgccggggctccagccgctgcacgacgacgtcaaataccacgcagtcctgaaccaccaccagcagcaggtcatcacgctcctcgaccccgacgcgccaggggcggcggcgtccccggaccaccagctcaagtggagcgacttccttgccgacgacgccgccgccttcgaggcggcgccgcaggtggttcttggtcagtaccaggaggccgcggtggctggtggcggagcagcgtatggagacactgatagtattgcagccgatggtgtcggcgggggcggggaggatagcgcagcgtcagcgttcatcgacgcgatgctggacagcgacaaaaagatgggcgtggaccagctcatcgccgacctgctcgccgacccggcatactactacggcggaggctcttcctcttcgacgtcggagctggggcggggcggttga

SEQ ID NO: 2 - DNA, 1751 bp, Triticum aestivum
  source 1..1751: mol_type = genomic DNA
gcgacgtgcccagggcgcggtggccggtggatcgcaggggctgtcggttgggatgcatgggacggagagtggagcgtccaaactatactttactccatgggagggaactgggcgtcggggtcggtcggtcttcacgtgtcccgggaatgggaagtgcaactctgtgtggtgctgcatctgatgctcgtattgctgatgccatgtgtatccacaggagagatgctgcagcgtagctgctttggaagcatttctcggttctcagcgttggaagcataagataagtaacgcacggaagtaagttgaacacaaaatcagagtggcgcagcggaagtgtggtgggcccataacccataggtcccaggatcgaaacttggctctaatttgattttttgaatttttcttttgaatttgatatttctatctctttattttcttggattaaacttcgtttcttttttctcttcgcgaatggcttactctactttctgttctccttcaaatattgatcatctacacactttcctttacatgaagctttttacctgacgaactataagaacaagtagtacaaactgttaacgccacaccagaaatgctcggtgcttcttctctgaaaccaacacgagacgctgaaaaaggcattccaaaaaaaaaaacacgagacgctgaaaacagcatgcattttctccttgtaataccacaactcgaaacctggctctgatatgattttttgaatttttcttttggatttgatatttctatctgtttattttcctggatcgaaacttggctctgatatgattttttgaatttttcttttggatttgatatttctatctgtttattttcctggattaaacttcgtttcttttttctcttcgcgaatggcttactctactttctgttctcctccaaatattgatcatctacacactttcctttacacgaagctttttacctgacgaaccataagaacaagtagtacaaactgttaacgccacaccagaaatgctcggtgcttcttctctgaaaccaacacgagacgctgaaaaaggcattaaaaaaaacacgagacgctgaaaacagcatgcattttctccttgtaataccacaactgttaaggatcacatgtgcggcgccaacacaactgtcaagacgatcgtacaaacgcgatgagcagaggcgcctgtgatatgatcgcatcgccagctcccaggtgaaaaccgattccgcggcgctgatgtcgtgaaaatcatcatgtccaactcgtgaaaatatttgaaacagcccccggcccctccaccctcacacacggcgtttgattagattagatcggattgtattaacccaccaacccgcatctgcatacatatttgatccctccctgattaagcggttgatttgccggagggaaacagttgcaggcgtgccataaactagggttaaagaagcgtttccatcggatcgtcactaacccatgccatgccatcactagttaaatgaggaggggggaggtcatgcttccgtcgcgtgcccctccaccatctggacatgttgttgccgtgcagtgcccctctctctctcgccggagcgccccgccccctatatgtacgccggtgccaccctgcagtgtgatctcgtcggggggcaccggaggttggtgcggtgctggggttctcgagctatattatctatcaccgtttggctgcttgttgtagaaggggagggggaggagggtggtgccggccggg

SEQ ID NO: 3 - DNA, 923 bp, Triticum aestivum
  source 1..923: mol_type = genomic DNA
atggggcggccgccgtgctgcgacaaggccaacgtgaagaaggggccgtggacggcggaggaggacgccaagctgctggcctacacctccaaccatggcaccggcaactggacctctgttccccagagggcagggctgaagcggtgcgggaagagctgcaggctgaggtacaccaactacctgaggcccaacctcaagcacgagaacttcacgcaggaggaggaggagctcatcgtcaccctccatgccatgctgggaagcaggtggtctctgatcgcgaaccagctgccggggcggacggacaacgacgtcaagaattactggaacaccaagctgagcaagaagctgcggcagcggggcatcgaccccatcacccatcgccccatcgccgacctcatgcagagcatcggcaccctccccatccgcccgccgcccagcgccgcgggtgcctcctcgtcctcctacatccccgtgaacccagcggcggcgccggggctccagccgctgcacgacgacgtcaaataccacgcagtcctgaaccaccaccagcagcaggtcatcacgctcctcgaccccgacgcgccaggggcggcggcgtccccggaccaccagctcaagtggagcgacttccttgccgacgacgccgccgccttcgaggcggcgccgcaggtggttcttggtcagtaccaggaggccgcggtggctggtggcggagcagcgtatggagacactgatagtattgcagccgatggtgtcggcgggggcggggaggatagcgcagcgtcagcgttcatcgacgcgatgctggacagcgacaaaaagatgggcgtggaccagctcatcgccgacctgctcgccgacccggcatactactacggcggaggctcttcctcttcgacgtcggagctggggcggggcggttg

SEQ ID NO: 4 - protein, 307 aa, Triticum aestivum
MGRPPCCDKANVKKGPWTAEEDAKLLAYTSNHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLPIRPPPSAAGASSSSYIPVNPAAAPGLQPLHDDVKYHAVLNHHQQQVITLLDPDAPGAAASPDHQLKWSDFLADDAAAFEAAPQVVLGQYQEAAVAGGGAAYGDTDSIAADGVGGGGEDSAASAFIDAMLDSDKKMGVDQLIADLLADPAYYYGGGSSSSTSELGRGG

SEQ ID NO: 5 - DNA, 1530 bp, Triticum aestivum
  source 1..1530: mol_type = genomic DNA
atggggcggccgccgtgctgcgacaaggccaacgtgaagaaggggccgtggacggcggaggaggacgccaagctgctcgcctacacctccaaccatggcaccggcaactggacctctgttccccagagggcaggtcggaaccctccccccggccggccggatcgacgtccgtcatggaaatggtgttggtcttgcatggtttctggctgatgtgtgttgcatcatggatgcgtgcgtgctgcagggctgaagcggtgcgggaagagctgcaggctgaggtacaccaactacctgaggcccaacctcaagcacgagaacttcacgcaggaggaggaggagctcatcgtcaccctccatgccatgctgggaagcaggtgcgtctctctgtgtagtacatgcatgtttctgccatcagttactttctcgattttgtcatcaagttttcggtgaatgcgaaactttgctatggaccgactgctgtttctgtcatcattcctgctataaaccttgcatccatatactctagcgttcagatcttatagatttatggaagttctgaatatctcacgttggtgtatcagacaattgctcatataccggaaatcacgtcctacttctattcaagaatttacttcagtcgttcgttcaaaaatagaaagaaatgtagtttagtccagtgaccagtctcgtgtctacgtacggttcttctgaatatatatattctgtttcctggtagaatgtagtgaaaagctgaaaattcggtgaagaacatatctgaatagctgtcttaaattgatagtgtttgacagaaaatactccacactgagcgtaacgtgaaagtgcgtgtacgtgttgggtgcatgcaggtggtctctgatcgcgaaccagctgccggggcggacggacaacgacgtcaagaactactggaacaccaagctgagcaagaagctgaggcagcggggcatcgaccccatcacccaccgccccatcgccgatctcatgcagagcatcggcaccctcgccatccgcccgccaccgagcgccgcgggtgcctcctcctcctcctacctccccgtgaacccaccggcggcgccggggctccagccgctgcacgacgacgtcaaataccacacagtcctgaaccagcagcagcagcaggtcatcacgctcctcgaccccgacgcgccaggggcggcggcgtccccggagcaccagctcaagtggagcgacttcctcgcggacgacgccgcggccctcgaggcggcgccgcaggtcgttcttggtcagtaccaggaggccgcggtcgctggtggcggagcacacgcgtatggcgacactgacagtactgcagccgatggtgtcggcgggggcggggacgatagcgcagcgtcagcgttcatcgacgcgatgctggacagcgacaagaagatgggcgtggaccagctcatcgccgacctgctcgccgacccggcatactactacggcggaggctcttcctcttcgacgtcggagctggggtggggctgttga

SEQ ID NO: 6 - DNA, 1212 bp, Triticum aestivum
  source 1..1212: mol_type = genomic DNA
gcgacgtgcccacggcgcggtggccagtggatcgcaggggttgtctgtggggatgcccgggacggcgagtgccgaaactatactttactccatgggaattgagcgtcggggtcggtcggtcttcacgtgtcccgggaatgggaagtgcaactctgtgagcaacgggcgggtgggggtgctgcatctgaggctcgtaatgctggtgccatgtctatccacacgagagaagctgcagcgtacagtgtgaagcctgataggttggttgcgatgcttagcgttggaagagaaactactactagtaccgtacgggtctacgttagacacaaaaatcagagtggcgcagcggaagcgtggtgggcccataacccacaggtcccaggatcgaaacctggctctgatatgattatttttttgaatttttcttgattagatttgatacttctatctgtttcttttccctggattgagcttcgtttttttccctctctgcccgaatggcttgctcggtgcttcttcttccttcttctctaaaaccaacacgagacgctgagacagcatgcatgcattttctcctcgtaatactacaaactgtcaaggatcacatgtgcggcgccaacacaactgtcaagacgatcgtacaaacgcgatgagcagaggcgcctgcgatatgatcgcatcgccagctcccaggtgaaaaccgattccgcggcgctgatgtcgtgaaaatcatcatgtacaactcgtgaaaatatttgaaacagcccccggccctccaccctcacacacagcgtttgattagattagattagattaacccaccaacccgcatctgaatatttgatccctccctgattaagctgttgatttgccggagggaaacagttgcaggcgtgccataaactagggttaaagaagcgtttccatcggatcgtcactaacccatgccatcactagttaaatgaggaggggggaggtcatgcttccgtcgcgtgcccctccaccatctggacatgttgttgccgtgcccccatctctctcgccggagcgccccggccccttatatgtacaccggtgccaccctgcagtgtgcgatctcgtcggggggcaccggaggttggtgcagtgctggttctcgagctatacatatatatatatctatcaccgtttggctgcttgtagaaggggaggggaaggagggtggcgccggcctgg

SEQ ID NO: 7 - DNA, 927 bp, Triticum aestivum
  source 1..927: mol_type = genomic DNA
tcgttcttggtcagtaccaggatgccgcggtcgctggtggcggagcacacgcgtatggcgacactgacagtactgcagccgatggtgtcggcgtgggcggggaggatagcgcagcgtcagcgttcatcgacgcgatgctggacagcgacaagaagatgggcgtggaccagctcatcgccgacctgctcgccgacccggcatactactacggcgggggctcttcctcttcgacgtcggatctggggtggggctgttgaatggggcggccgccgtgctgcgacaaggcgaacgtgaagaaggggccgtggacggcggaggaggacgccaagctgctcgcctacacctccaaccatggcaccggcaactggacctctgttccccagagggcagggctgaagcggtgcgggaagagctgcaggctgaggtacaccaactacctgaggcccaatctcaagcacgagaacttcacgcaggaggaggaggagctcatcgtcaccctccatgccatgctgggaagcaggtggtctctgatcgcgaaccagctgccggggcggacggacaacgacgtcaagaactactggaacaccaagctgagcaagaagctgcggcagcggggcatcgaccccatcacccaccgccccatcgccgacctcatgcagagcatcggcaccctcgccatccgcccgccaccgagcgccgcgggtgcctcctcctcctcctacctccccgtgaacccagcggcggcgccggggctccagccgctgcacgacgacgtcaaataccacgcagtcctgaaccagcagcagcagcaggtcatcacgctcctcgacgccgacgcgccaggggcggcggcgtccccggaccacccgctcaagtggagcgacttcctcgccgacgacgccgccgccttcgaggcggcgccgcagg

SEQ ID NO: 8 - protein, 308 aa, Triticum aestivum
MGRPPCCDKANVKKGPWTAEEDAKLLAYTSNHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLAIRPPPSAAGASSSSYLPVNPAAAPGLQPLHDDVKYHAVLNQQQQQVITLLDADAPGAAASPDHPLKWSDFLADDAAAFEAAPQVVLGQYQDAAVAGGGAHAYGDTDSTAADGVGVGGEDSAASAFIDAMLDSDKKMGVDQLIADLLADPAYYYGGGSSSSTSDLGWGC

SEQ ID NO: 9 - DNA, 1530 bp, Triticum aestivum; identical to SEQ ID NO: 5
  source 1..1530: mol_type = genomic DNA

SEQ ID NO: 10 - DNA, 1102 bp, Triticum aestivum
  source 1..1102: mol_type = genomic DNA
cgacgtgcccacggcgcggtggccggtggatcgcaggggttgtcggtgggaatgcctgggacggagagcgtccaaactgtagtagtagtactttactccatgggaactgggcgtcggggtcggtcggtcttcacgtgtcccggggatgggaagtgcaactctgtgtgctgcatctgatgctcgtattgctggtgccatgtgtatccacaggagagatgctgctttgggagtctagagtgtgtgtcaagcggcattgctcggtgctcagcgttggaagcataagacaagtaccgcacgagagatgctgtttcttttcctggatttaacttcgtatcttcccaagtggcttactgactcagtagtacaaaacgttacacgccaaaccacaaaatgctcggtgcttcttctcccttcttctctgaaaccaacacgagacgctgagacagcatgcattttctcctcgtaatactgcaactgtcaagcatcacatctccggcgccaacgcaactgtcaatacgatcgtacaaaagcgatgagcagaggcgcctgcgatatgatcgcatcgccagctcccaggtgaaaaccgattccgcggcgctgatgtcgtgaaaatcatcatgtccaactcgtgaaaatatttgaaacagcccccggcccctccaccctcacacacagcgtttgattagattagatcggattaacccaccaacccgcatctgaatatttgatccctccctgattaagcggttgatttgccggagggaaacagttgcaggcgtgccataaactagggttaaagaagcgtttccatcggatcgtcactaacccatgcatgccatcacttgttaaatgaggaggggggaggtcatgcttccgtcgcgtgcccctccaccatctggacatgttgttgccgtgcagtgcccctctctctctcgccggagcgccccggcccctatatgtacaccggtgccaccctgcagtgcgatctcgtcggggggcaccgcaccggaggttggtgcagtgcgggggttctcgagctatatatctatcaccgtttggctgcttgtagaaggggaggggaaggagggtggcgccggccggg

SEQ ID NO: 11 - DNA, 927 bp, Triticum aestivum
  source 1..927: mol_type = genomic DNA
atggggcggccgccgtgctgcgacaaggccaacgtgaagaaggggccgtggacggcggaggaggacgccaagctgctcgcctacacctccaaccatggcaccggcaactggacctctgttccccagagggcagggctgaagcggtgcgggaagagctgcaggctgaggtacaccaactacctgaggcccaacctcaagcacgagaacttcacgcaggaggaggaggagctcatcgtcaccctccatgccatgctgggaagcaggtggtctctgatcgcgaaccagctgccggggcggacggacaacgacgtcaagaactactggaacaccaagctgagcaagaagctgaggcagcggggcatcgaccccatcacccaccgccccatcgccgatctcatgcagagcatcggcaccctcgccatccgcccgccaccgagcgccgcgggtgcctcctcctcctcctacctccccgtgaacccaccggcggcgccggggctccagccgctgcacgacgacgtcaaataccacacagtcctgaaccagcagcagcagcaggtcatcacgctcctcgaccccgacgcgccaggggcggcggcgtccccggagcaccagctcaagtggagcgacttcctcgcggacgacgccgcggccctcgaggcggcgccgcaggtcgttcttggtcagtaccaggaggccgcggtcgctggtggcggagcacacgcgtatggcgacactgacagtactgcagccgatggtgtcggcgggggcggggacgatagcgcagcgtcagcgttcatcgacgcgatgctggacagcgacaagaagatgggcgtggaccagctcatcgccgacctgctcgccgacccggcatactactacggcggaggctcttcctcttcgacgtcggagctggggtggggctgttga

SEQ ID NO: 12 - protein, 308 aa, Triticum aestivum
MGRPPCCDKANVKKGPWTAEEDAKLLAYTSNHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLAIRPPPSAAGASSSSYLPVNPPAAPGLQPLHDDVKYHTVLNQQQQQVITLLDPDAPGAAASPEHQLKWSDFLADDAAALEAAPQVVLGQYQEAAVAGGGAHAYGDTDSTAADGVGGGGDDSAASAFIDAMLDSDKKMGVDQLIADLLADPAYYYGGGSSSSTSELGWGC

SEQ ID NO: 13 - DNA, 1965 bp, Zea mays
  source 1..1965: mol_type = genomic DNA
atggggaggccgccgtgctgcgacaaggcgaacgtgaagaaggggccgtggacgccggaggaggacgccaagctgctggcctacacctccacccatggcaccggcaactggaccaacgtgccccaacgagcaggtgatcgtgccgccgtgcaccatgcattttgttgttttgtttgtggctcatgacgaggcgctggcggacggtgcggcatgatgtgatatgatgcgcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtacgcagtacagcgctgcagaatcatgttcatggccgtttgctttgattaattccacacaacatgcatgcatgcattcgcatcatccttcagcttcctcacctaggaaccggatagatccttcgtagtgctgcagataaatccgatttatcttctatctcaatgcggttttgaaactaagtatatcacattagatagtgttaattgctgaactgaagaagatctgaattatgaaagacacgtccgatcctgcagctcttacaaaggttttctctttttttaagaaaaaaaatctgcctccatttaccgtaggtgattcttcctggacatttttgttccgcggcaaattaaatagtaattgaacctatgtttcacatgagaaaattgctagtaatcgggtgtttggaaatgattctgaatcttgcggacttaaatctgaaaccaatcgtcccaatgcaattcgctagagcaattgatctgttcatttccaatcagtcaatcaccaagccctagaaaacggacagctagttcagtagttcccgcatcagcgccattgctgatggatcgaacagctgacgcgaatgaaaacgacatgacaccgtcggggagatcgttggatgagttccgagcgataacgaactgtacgggcagtgacatacacaatgcgtgcgcgcatgcaaagttgattggaatccaatgcgtccagctgataggagtatttacactacagatacactcatagttgctagggtaggtgatcttgagatgcatcttgatccctcgctagttagtactattcatgctatttgctgcagttaattaacgggtccggcctgcaatggaaattgtagtgcgctagaccgcgcgctgctgatctgggccacgaactgcgcgcgtttgcatgcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgattaa

SEQ ID NO: 14 - DNA, 966 bp, Zea mays
  source 1..966: mol_type = genomic DNA
tccatccggaaacgaaaccgctagagctaaggtctcaatcaacacagtgtcgagtgtggacaagttggtgctgctggactgctggcacatatacgaaaacgaaagcaggtcaggtggagagcagaggaggagccgtgccgcaggcagctattttatagctgctcgacgcgggtgctacacgcgtccagtgggaacggctgcctttctcggagcgcgtagcctggcgcgcgctggttggctccactctgcggcgttttttcaatttgtttttttactcctctttctgcggggaggcggcgagatgtcgggtggacggtggatcggcaaacgaggggcctgtcggtgtggacggtggagatgcactaagccttctttgagcaggtcacatcttttgcaagttcatggtgcccagtacgtagtggacagcagagaagctccgggcggggccaatgcaaaaccgatctggcggcgtcgatgtcgtgaaaaccgtcatgcccaactcgtgaaaattttaaaccccagcatcaccaggccgctagctccgtctctcaacgataagattacccacaccaacccgcgctcgccctaatgagctgttgatttgccggagggaagcagttgcgcgcgcgctctactatactgtcgccgccgccataaacaaagagggaaccagcgtctcttccctaatctaaccatctcctgcgtgattgacactaaccatgccgtggctagttaaatgacgggggcggggtcacgccttcgttgcgtgcctccacctccccccctcggcgcccccaacgacatgttgttaccgtggctgtggcagccggccggtctccttctccatccatatgtaccggcagcatcgtatcacctttttttctgcagcggtgatctcatctaggcgtcggtcagagctctctcgagctcgccagcggtggttggtcgtcgtcgtcgtcgtcgtcg

SEQ ID NO: 15 - DNA, 959 bp, Zea mays
  source 1..959: mol_type = genomic DNA
atggggaggccgccgtgctgcgacaaggcgaacgtgaagaaggggccgtggacgccggaggaggacgccaagctgctggcctacacctccacccatggcaccggcaactggaccaacgtgccccaacgagcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgatta

SEQ ID NO: 16 - protein, 319 aa, Zea mays
MGRPPCCDKANVKKGPWTPEEDAKLLAYTSTHGTGNWTNVPQRAGLKRCGKSCRLRYTNYLRPNLKHENFTQEEEDLIVTLHAMLGSRWSLIANQLPGRTDNDVKNYWNTKLSKKLRQRGIDPLTHRPIADLMHSIGALAIRPPQPATSPNGSAAYLPAPALPLVHDVAYHAAGMLPPTPAPPRQVVIARVEADAPASPTEHGHELKWSDFLADDAAAAAAAAAEAQQQLAVVGQYHHEANAGSSSAAAGGNDGCGIAVGGDDGAAAFIDAILDCDKETGVDQLIAELLADPAYYAGSSSSSSSSSGMGWAGMGLLNAD

SEQ ID NO: 17 - DNA, 2268 bp, Oryza sativa
  source 1..2268: mol_type = genomic DNA
atggggcggccgccgtgctgcgacaaggcgaacgtgaagaaggggccgtggacggcggaggaggacgccaagctgctggcgtacacctccacccacggcaccggcaactggacctccgttcctcagcgagcaggtcgtgaaaatcaacgatatcgatcgccgttgctgctgctgccgatgggatcgagattgcaattagagacgcatttgatcacatcagatcagatcaaaatatggtgtcattgtttatcacttttttttcccttttgttttgttctgtaaaaattgcatggggggtgatgtgggtgcaggtttgaagaggtgcgggaagagctgcaggctgaggtacaccaactacctgaggcccaacctgaagcacgagaacttcacgcaggaggaggaagagctcatcgtcaccctccacgccatgctgggcagcaggtatatcatcgtgttacgctcagcttgtcccgttatctcgtcatttcagacgtcaccatgcttttgcatgtgtggtgagtggtgtaatacactccgtccgttggaccctcctgcttgattgatctgtcgttgctgttttcgaattgataatccacgtgctagtttgtcatcgactcagatttggcgaatcggtgttcgtgatttgtatctgatttctgcgtgtaatgctaagcatgttgcgaactaccttgatcctataataagcgtaattataggtttcagtgtccaactttatctaacaatcttatttaaaatattttaatgattagtattttttttattgttattagatgataaaatatgaacagtactttatgcgtgtcttaacttttaaaaaaaattcataaactttttaaataagacggacaattaaatgttgggcacgtaaacttatagctgcacgtataataagacggagggagtaattcccttgaatcctaacatgagataaatagctagtgcacataattggctgctctaatgtactttggcagtatgatgctgatgcaattagcatttacatttaatgttctaaaaattttatggaatcaacctagatgtgaacgaaaatggcatgacaaaataatcaagatccctgagcggtaacaaactgatacacacacaatgcatgtaacgccactattcacagttaattagtactgtactactaattaacttagttaatactagcactagttattttacactctaaacaaatgctgcgagatcatctctaccctcaatcagaaatgggagctagtttgacacatcaacacacaatttggactgagatcattgagcatgcatgtaatttctactccatccgtttcagattataagatattttaactttgatcaaagttaaactgttttaaatttgactaagtttatagacaaatatagtaatatttacattaccaaattagttttattaaattaataataaaatatatttttataataaatttatcttaagtcaaaaatattattattttttctatcaaattaatcaaacttgaagcagtttaacttttatctaagtcaaaaacatccgagggagtataaccaaagttgtgcgcgtgcatcaatcaaatgcgcgtgattggcaggtggtcgctgatcgcgaaccagctgccggggaggacggacaacgacgtgaagaactactggaacaccaagctgagcaagaagctgcggcagcgtggcatcgaccccatcacccaccgccccatcgccgacctcatgcagagcatcggcacgctcgccatccgcccccctcccgccgccggcgccgcgccgccgccctgcctcccggtgttccacgacgcgccgtacttcgccgccctgcagcatcagcatcagcagcagcaggtcgtcacgcacgtcgacgccgacgcgcccgcgtcgcccgactcgcagcatctgcagctcaactggagcgacttcctcgccgacgacgccgcggggcacggcgccgacgcgccggcgccgcaggctgctctcggccagtatcaggaggggtcagcaccggcggcgactgccgtcgtgggcggaggccgcgcgttcggtgacgtcgacggtgcatcagctggcgtcggcgccggcacggacgacggcgccggggctgcgtcggcgttcattgacgcgatcctcgactgcgacaaggagatgggggtggaccagctcatcgccgagatgctcgccgacccggcatactacggcggcggtggcggctcctcctcgtcggagctcggctggggttgctaa

SEQ ID NO: 18 - DNA, 1316 bp, Oryza sativa
  source 1..1316: mol_type = genomic DNA
tggaacgcatggcaagaagggtagtagtacgccgtacgatcagacggttacgcgtgatgtaacgtgtcgacgatccatgatccatcgactaggaggtcatcaccggggcccacctgccccccgggagggaggttgcgttggtgggcccgggggagtcaaacgacggcggagatgagacggagagggccccgccgttatgctgtcgggctgtcggcgcttcacacagtggccgtccgtacgtgatgtcgcctcctccagccgtctccaagtacagctatactagtagagtagtatactactgctcctatactgtacagtataccccgtactgtactagtggcaatatcactcaaaacacatggagcattatgtatacatacaaccatcatgaatatatattcttctagaaacgaaaaaagcatgcacatcgcccctattttggggagttagttaattagaatattcagctgataggaatctttaaaagaatcggataattaattaaccataatttctgtcatgcagggtatcaaatgtaccacattaaatttttctagcaatgtaaaatctatgcatgcaccacactggacagcgaaatatatactcccttcgtactcataaagggaatcgttttggacagtgacacggtctccaaaacacaactttgactttttgtttctataaaaatatttattgaaaagtgatatatgtatacttttatgaaagtatttttcaagacaaatctattcatatattttttatattttcaaattcaataatttaaaaattattcatgatttatattctcaaggtttgacttaaatattatcctaaacgattttctttatgagtacggagggagtatacttacaattttgtacctctcgagtacgataaaatctctctccagattttgcgcgagaatatctgaacggtttgtagctgcattatctagaagatctcttgaaaatgaacatagttcatatattacctcatgtatgtggtgctatatatatatatgtttcactggatggttaattacttctgggaaactgttttaacatgcaacatgtactagctagctagctccatttctcttcattccattccagagagctcctctatttcttttactaatctttttcccctatcaaaaagccaccagctttctagtaagcaacactagtcactttaacctcctcccttgcttttgcttactacaccttgcatctctctctggtaaccgtatcgtggtggaaggaaaggaagaaaggagtgtactgggtagctcagctcagctcagctaggcagtggcc

SEQ ID NO: 19 - DNA, 579 bp, Oryza sativa
  source 1..579: mol_type = genomic DNA
atgtcagagcgtgtgttcgccgagctcgcgaccatccactaccaaaaaagccttccatgtcgccactcctttgacccccctcgcaccacaccaattctccatctatatatcatccaccttcttcttcctcctctcattgccattgtgtgtttgtgttacattgcaatcgtgccatttgaagaagaggaggagaggatgaggatgcaggtggtggagacggcggcggtggaggaggaggaggcggcggcggcgatgatgtcggtgtacgagagggtggcgaggatggcgagcgggaacgcggtggtggtgttcagcgcgagcgggtgctgcatgtgccacgtcgtcaagcgcctcctcctcggcctcggcgtcggccccgccgtctacgagctcgaccagctcgccgccgccgccgacatccaggccgcgctgtcgcagctcctcccgccgggccagccgccggtgcccgtcgtgttcgtcggcggcaggctcctcggcggcgtcgagaaggtgatggcgtgccacatcaatggcaccctcgtccccctcctcaagcaggccggcgccctctggctctga

SEQ ID NO: 20 - protein, 306 aa, Oryza sativa
MGRPPCCDKANVKKGPWTPEEDAKLLAYTSTHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLAIRPPPAAGAAPPPCLPVFHDAPYFAALQHQHQQQQVVTHVDADAPASPDSQHLQLNWSDFLADDAAGHGADAPAPQAALGQYQEGSAPAATAVVGGGRAFGDVDGASAGVGAGTDDGAGAASAFIDAILDCDKEMGVDQLIAEMLADPAYYGGGGGSSSSELGWGC

SEQ ID NO: 21 - DNA, 414 bp, Triticum aestivum
  source 1..414: mol_type = genomic DNA
atgttgaggatgcagcagcaggtggagggcgtggtgggcggcggcatcgtggccgaggcggaggaggcggcggtgtacgagcgggtggctcgcatggccagcggcaacgccgtggtcgtcttcagcgccagcggctgctgcatgtgccacgtcgtcaagcgcctcctgcttggcctgggagtcggccccaccgtctacgagttggaccagatgggcggcgccgggcgggagatccaggcggccctggcgcagctgctgccccccggacccggcgccggccaccaccagcagccgccggtgcccgtggtgttcgtcggcgggaggctcctgggaggcgtggagaaggtgatggcgtgccacatcaacggcacgctcgtcccgctcctcaaggacgccggcgcgctctggctctga

SEQ ID NO: 22 - DNA, 1052 bp, Triticum aestivum
  source 1..1052: mol_type = genomic DNA
gttatgcatgcggccggggtctggtgggggaatcgctcacgtgaaggcaatgtgggacgtgaaggaggtgacgatgggcgtgacggcgtttggtggcagtggggccgaggagtcaaacggcggctgagggggacgtcgtgctgtcggcttgtcggctgccgtgacgatcgacgcttgtcttgtcacgtcgtgtccatgcttcgttcccttcctccctccctccctccgtatagtcggcgttcaaattcttggcctgcagatacatgtaaattcctaagtttgaaggcacattggttcattataaagaaaatagttttgctgaccgagacttcgttatatctcagttgatgttatttttctttaattttgcatgaagattcgtacaaaaaatatattttcagttttttttctctttttcttcttacatactactgtatatcatttgagtgagacttggttaagtctcagtcgactgagacctaaccacaccctaaaagaaaagagtactattaattgattttgctgatgtgccacaacatgagtagattaggcggccgtgcatgcatgctaggatcggctcgccatgccatgcatccatccatctttcggccatccctttgcttttttccttctcctcgtctacggacaaaagctactaccgcatagctctttagcttgctcctcgctttactctctatctcatctcttgtaaccgtagtggaatggggtggagtagtggtggtcagagtgtgccgggttcgttgcggagctcgcgacaggctctgccaaaagcgatccatgtctctttgactccccacaccccacccccgtatactcccttcccttctcccatcacacttccattcgagagacacacacacacacccttcgacctgtccgcctctcctatataaacgcacgtccaccgctccctgttttcttcatcctcgttcccttgcactgcgccaacaaacaattaactacactctcactagctagctaggcgaggctaggccggcgagggagaaggagaaggagaagtaggcgccaag

SEQ ID NO: 23 - DNA, 414 bp, Triticum aestivum; identical to SEQ ID NO: 21
  source 1..414: mol_type = genomic DNA

SEQ ID NO: 24 - protein, 137 aa, Triticum aestivum
MLRMQQQVEGVVGGGIVAEAEEAAVYERVARMASGNAVVVFSASGCCMCHVVKRLLLGLGVGPTVYELDQMGGAGREIQAALAQLLPPGPGAGHHQQPPVPVVFVGGRLLGGVEKVMACHINGTLVPLLKDAGALWL

SEQ ID NO: 25 - DNA, 414 bp, Triticum aestivum
  source 1..414: mol_type = genomic DNA
atgttgaggatgcagcagcaggtggagggcgtggtgggcggcggcatcgtggcggaggcggaggaggcggcggtgtacgagagggtggctcgcatggccagcggcaacgcggtggtcgtcttcagcgccagcggctgctgcatgtgccacgtcgtcaagcgcctcctgcttggcctgggagtcggccccaccgtgtacgagttggaccagatgggcggcgccgggcgggagatccaggcggccctggcgcagctgctgccccccggacccggcgccggccaccaccagcagccgccggtgcccgtggtgttcgttggcgggaggctcctgggcggcgtggagaaggtgatggcgtgccacatcaacggcacgctcgtcccgctcctcaaggacgccggcgcgctctggctctga

SEQ ID NO: 26 - DNA, 1044 bp, Triticum aestivum
  source 1..1044: mol_type = genomic DNA
acacgctctacatgcatacatacatacatacatacatacggaggctgtcaatacggggactctattaccgttgctctactgcccgcaaccgactatggcccgttgtgctgatctattggttggttgtgtcgtgttaggcgttggtgacgaagacccaagagcgatggacggttatgcatgccgccggggcctggtggggggaatcgatcacgtgaaggcaatgtgggacgtgaaggaggtgacgatgggcgtgacggcgtttggtggctgtggggccgaggagtcaaacggcggctgagggggacgtcgtgctgtcggcttgtcggctgccgtgacgatcgacgcttgtcttgtcacgccgtgtccatgctccgttccctccctccgtatagtcggcgttcaaattcttgtcctgcagatacatgtaaattcctacagtagtactactactttagaattcgcctgtaaatcaagatggacattattttctgagagcgttgaacgcccattggttcattatattataggagtataaagaaaaggggtaattgattttgctgatgtgccacgacaggagtagattaggcggctgtgcatgcatgcatgctaggatcgcctcgccatgccatgccatgccatgcatccatccatctttcggccatcccttcgctttttccttctcctcgtctacgcacaaaagctaccgcatagctctttaacttgctcctcgctttactctcatctcttgtaaccgtagtggaatggggtggagtactagtggtcagagtgtgccgggttcgttgcggagctcgcgaccggctctgccaaaagcgatccatgtctctttgactccccacaccccaccccttcccttctcccatcacacttccgttcgagacacacacacacaccctgtccgcttctcctatcctatataaacgcacgtccaccgctccctgttttcttcatcctcgttcccttgcactgcgccaacaaacaattaactacactctcactctcactagctagctaggctaggccaag

SEQ ID NO: 27 - DNA, 414 bp, Triticum aestivum; identical to SEQ ID NO: 25
  source 1..414: mol_type = genomic DNA

SEQ ID NO: 28 - protein, 137 aa, Triticum aestivum; identical to SEQ ID NO: 24

SEQ ID NO: 29 - DNA, 414 bp, Triticum aestivum
  source 1..414: mol_type = genomic DNA
atgttgaggatgcagcagcaggtggagggcgtggtgggcggcggcatcatggcggaggcggaggaggcggcggtgtacgagcgggtggctcgcatggccagcggcaacgcggtggtcgtcttcagcgccagcggctgctgcatgtgccacgtcgtcaagcgcctcctgcttggcctgggggtcggccccaccgtctacgagttggaccagatgggcggcgccgggcgagagatccaggcggcgctggcgcagctgctgccccccggacccggcgccggccaccaccagcagccgccagtgcccgtggtgttcgtcggcgggaggctcctgggcggcgtggagaaggtgatggcgtgccacatcaacggcacgctcgtcccgctcctcaaggacgccggcgcgctctggctctga

SEQ ID NO: 30 - DNA, 1071 bp, Triticum aestivum
  source 1..1071: mol_type = genomic DNA
aagattgggacaaactttttcgctcggaggaagtatgatagacgctctacatacatatatacgtacggaggctgtcaatacggggactattaccgttgctctactgcgcgcaaccgactatggcccgttgtgctgatctattggttggttgtgtggtgtgagccgttggtgaggaagacccaagagcgatggacggttatgcatgccgcgggggcctggtgggggaatcgatcacgtgaaggcaatgtgggacgtgaaggaggtgacgatgggcgtgacggcgtttggtggcagtggggccgaggagtcaaacggcggctgagggggacgtcgtgctgtcggcttgtcggctgccgtgacgatcgacgattgtcttgtcacgccgtgtccatgctccgttccctccctccctccctccgtatagtcggcgttcaaattcttggcctgcagatacatgtaaattcctagtacttagaattcgcctgtaaatcgagatggacaatattttctgagagcgttaaaggcccattggttcattataaagaaaaggagtaactgattttgctgatgtgccacaacataagtagattaggcggccgtgcatgcatgcatgctacggagtaggatcggctcgccatgccatgcattgcatccatctttcggccatccctttgctttttccttctcctcgtctacgcacaaaagctaccgcatagctctttaacttgctcctcgctttactctcatctcttgtaaccgtagtggaatggggtggagtagtggtggctagagtgtgccgggttcgttgcggagctcgcgaccagctctgccaaaagcgatccatgtctctttgaccccccacaccccacccccgtatactcccttcccttctcccatcacacttccattcgagacacactcacccttcgacctgtccgcctctcctatataaacgcacgtccaccgctccctgttcctcatcctcgttcccttgcactgcgccaacaaacaattaactacactctcactagctagctaggctaggccggggagggagaaggaggcgccaag

SEQ ID NO: 31 - DNA, 414 bp, Triticum aestivum; identical to SEQ ID NO: 29
  source 1..414: mol_type = genomic DNA

SEQ ID NO: 32 - protein, 137 aa, Triticum aestivum
MLRMQQQVEGVVGGGIMAEAEEAAVYERVARMASGNAVVVFSASGCCMCHVVKRLLLGLGVGPTVYELDQMGGAGREIQAALAQLLPPGPGAGHHQQPPVPVVFVGGRLLGGVEKVMACHINGTLVPLLKDAGALWL

SEQ ID NO: 33 - DNA, 480 bp, Zea mays
  source 1..480: mol_type = genomic DNA
atgctgcggatggaggtgcagcagcagcagcaggagtcgggagtgagcggcggcgtggtggcggacgcggcggcggcatccggggcggatgccgcgccgacgacgacgacgatggtggccgcggcgccgcactcggcgtcggcgctggcggtgtacgagcgggtggcgcgcatggcgggcgggaacgcggtggtggtgttcagcgccagcggctgctgcatgtgccacgtcgtcaagcgcctgctgctgggcctcggcgtcggccccaccgtgtacgagctcgaccagatggccgccggcggcggcgggggcagggagatccaggcggcgctggcgcagctgctgccgccgggccagccgcccctgcccgtcgtcttcgtgggcggacgcctcctcggcggcgtcgagaaggtcatggcgtgccacatcaacggcaccctcgtcccgctcctcaagcaggccggcgcgctctggctctga

SEQ ID NO: 34 - DNA, 1132 bp, Zea mays
  source 1..1132: mol_type = genomic DNA
aattcgcgggacgtggcgttgtcggctccgtgtcggcggccgaaccaccacgaatcactgacgtatctcgtctcctctctcctctagactcccacgatacggccaacgaagtgtatgtacatatatacccatggtcatatggcaacaaacgccaacgccagcagagcactgcccggcggcctttttcccatctctctctctctctctgatggggtgtgcatgcctgactgactgatagatagatagatggtcaggtccgtctgatcctcatcggcctagctcaccccacgcgaaaaaagccactgctggctggcgcccagttgcgcttgcaacagtcactttaacgagctccgtccttgcgtttgccctcctcgctctgcccctgccgccgctgccgctgcgtggtggtgctggtgcatgaggcaggcaggcgtactagtgcatgcaattgcaatgcaaccgtaggagtgcgttgcgtaccctggtctgtccctgcggcctggcctgcccttgttcgttgcggatgcggggggtgccgggtgggtactgtactgtactactgggtagagagatactactagatagagagagagagaggtcggtcaccccgggcgcgggacacagcctctgcgaaaaagcgatccatgtcgcgcctagctttgacccggaacggatcccccaaccaggaaccagcagagcaggagggccaggccaccacctctcgccattccattcccggtcctagctagtcctgttctgttcctgtagcagtagcagtagctacggtactacgagtcctcctcgacgtcccaggcactactccactccacgcagcagcaggcagcgagcatctctcgaccagatgcatacaagctacaccctcctcggctccgatcctacccatgccggcccaggcggcctataaaagcgcacccccggcccgtcttcctcccactgcatgcccattgcccctcccccggccttcgccgtgccaacgacacacctcatcaccggccggaacattccacgaccgaagaaaccagtccctagctagtccacgcacgaccaacaaggcaggcgagcgacgacagtccaagcctccaagaagaagaagaagaagaagaagaagaag

SEQ ID NO: 35 - DNA, 480 bp, Zea mays; identical to SEQ ID NO: 33
  source 1..480: mol_type = genomic DNA

SEQ ID NO: 36 - protein, 155 aa, Zea mays
MLRMEVQQQESGVSGGVVADAAAAGSVAEAATTTMVAAAPHSASALAVYERVARMAGGNAVVVFSASGCCMCHVVKRLLLGLGVGPTVYELDQMAGGGGGREIQAALAQLLPPGQPPLPVVFVGGRLLGGVEKVMACHINGTLVPLLKQAGALWL

SEQ ID NO: 37 - DNA, 384 bp, Oryza sativa
  source 1..384: mol_type = genomic DNA
atgaggatgcaggtggtggagacggcggcggtggaggaggaggaggcggcggcggcgatgatgtcggtgtacgagagggtggcgaggatggcgagcgggaacgcggtggtggtgttcagcgcgagcgggtgctgcatgtgccacgtcgtcaagcgcctcctcctcggcctcggcgtcggccccgccgtctacgagctcgaccagctcgccgccgccgccgacatccaggccgcgctgtcgcagctcctcccgccgggccagccgccggtgcccgtcgtgttcgtcggcggcaggctcctcggcggcgtcgagaaggtgatggcgtgccacatcaatggcaccctcgtccccctcctcaagcaggccggcgccctctggctctga

SEQ ID NO: 38 - DNA, 1511 bp, Oryza sativa
  source 1..1511: mol_type = genomic DNA
tggaacgcatggcaagaagggtagtagtacgccgtacgatcagacggttacgcgtgatgtaacgtgtcgacgatccatgatccatcgactaggaggtcatcaccggggcccacctgccccccgggagggaggttgcgttggtgggcccgggggagtcaaacgacggcggagatgagacggagagggccccgccgttatgctgtcgggctgtcggcgcttcacacagtggccgtccgtacgtgatgtcgcctcctccagccgtctccaagtacagctatactagtagagtagtatactactgctcctatactgtacagtataccccgtactgtactagtggcaatatcactcaaaacacatggagcattatgtatacatacaaccatcatgaatatatattcttctagaaacgaaaaaagcatgcacatcgcccctattttggggagttagttaattagaatattcagctgataggaatctttaaaagaatcggataattaattaaccataatttctgtcatgcagggtatcaaatgtaccacattaaatttttctagcaatgtaaaatctatgcatgcaccacactggacagcgaaatatatactcccttcgtactcataaagggaatcgttttggacagtgacacggtctccaaaacacaactttgactttttgtttctataaaaatatttattgaaaagtgatatatgtatacttttatgaaagtatttttcaagacaaatctattcatatattttttatattttcaaattcaataatttaaaaattattcatgatttatattctcaaggtttgacttaaatattatcctaaacgattttctttatgagtacggagggagtatacttacaattttgtacctctcgagtacgataaaatctctctccagattttgcgcgagaatatctgaacggtttgtagctgcattatctagaagatctcttgaaaatgaacatagttcatatattacctcatgtatgtggtgctatatatatatatgtttcactggatggttaattacttctgggaaactgttttaacatgcaacatgtactagctagctagctccatttctcttcattccattccagagagctcctctatttcttttactaatctttttcccctatcaaaaagccaccagctttctagtaagcaacactagtcactttaacctcctcccttgcttttgcttactacaccttgcatctctctctggtaaccgtatcgtggtggaaggaaaggaagaaaggagtgtactgggtagctcagctcagctcagctaggcagtggccatgtcagagcgtgtgttcgccgagctcgcgaccatccactaccaaaaaagccttccatgtcgccactcctttgacccccctcgcaccacaccaattctccatctatatatcatccaccttcttcttcctcctctcattgccattgtgtgtttgtgttacattgcaatcgtgccatttgaagaagaggaggagagg

SEQ ID NO: 39 - DNA, 384 bp, Oryza sativa; identical to SEQ ID NO: 37
  source 1..384: mol_type = genomic DNA

SEQ ID NO: 40 - protein, 127 aa, Oryza sativa
MRMQVVETAAVEEEEAAAAMMSVYERVARMASGNAVVVFSASGCCMCHVVKRLLLGLGVGPAVYELDQLAAAADIQAALSQLLPPGQPPVPVVFVGGRLLGGVEKVMACHINGTLVPLLKQAGALWL

SEQ ID NO: 41 - DNA, 1926 bp, Triticum aestivum
  source 1..1926: mol_type = genomic DNA
atgagcagccccatggaggaagctcaccatggcatgccgtcgacgacgacggcgttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcatgtgctcaagaccaacttcaacaattaccccaaggtgaaactgaaagaacccctcagccttgtgaatttttttgccaaggttcagaagtttacactgacacaaatgtctgaaattgtacgtgtagggggaggtgtacaggtcctacatggacgtgctgctcggcgacggcatcttcaacgccgacggcgagctctggaggaagcagaggaagacggcgagcttcgagttcgcttccaagaacctgagagactttagcacgatcgtgttcagggagtactccctgaagctgcgcagcatcctgagccaggcttgcaaggccggcaaagtcgtggacatgcaggtaaccgaactcagtcccttggtcatctgaacattgatttcttggacaaaatttcaagattctgacgcgagcgagcgaattcaggagctgtacatgaggatgacgctggactcgatctgcaaggtggggttcggggtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtacgtgatcgtcgtcgtcaagctccggatcgctggtttgtgtaggtgccattgatcactgacacactagctgggtgcgcagatcaagcacgacatactgtcgcggttcatcgagctgggcgaggccggcggggacgacggcggcagcctgttcggggacgacaagggcctccgcgacgtggtgctcaacttcgtgatcgccgggcgggacaccacggccacgacgctctcctggttcacctacatggccatgacgcacccggccgtggccgagaagctccgccgcgagctggccgccttcgaggcggaccgcgcccgcgaggatggcgtcgcgctggtcccctgcagcgactcagacggcgacggctccgacgaggccttcgccgcccgcgtggcgcagttcgcggggctgctgagctacgacgggctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgcgttccgcaacgcgtcgccgttcaagttcacggcgttccaggcggggccgcggatctgcctcggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 42 - DNA, 1000 bp, Triticum aestivum
  source 1..1000: mol_type = genomic DNA
ctgttcggttattctttcattgtttgccatggagacatgaattatcttcatctgatacttgtcaaattagaagtgatcttcattagttgtacatcatccacagctgctacggtagtaccaacagaaaacggccagcttgaaagttttggaatgcaatgttagggagtactatttagtagagcatatatatgataaaagtgttctcatctgtggaacatatttatttggcagcactagatgcctcggcatattgcaaggtttttaatatttgcgatcttttctgtttcaagcttctaataaatagaaggtgaccactttcatcaaaattttcttctgtttagcttctgctacaaatttctaataaatatagaagggggaactttcagcaagattttttatatttgtgattttcaggctttttccatttagggagaacatcagagcaccccttgacagttgacaccccttcattcgaaatttctcaacttgttctgctttgacttcaaaaactgtttcactgaaagatgcactttgtattggttagtgcgggttcaataaagaccagatggaccataaccatggctccatggctccaactgtgaagatgacataatcacaacgctaactgtcatcaaacgcatcacctacatcccccgcaaaacgaaataaaaatgcatcagtgcatcacctacatttatagtaaaacagaaggaaaatgcagaatccatgacctagcttagcaccaagcacatactaacatacctagttatgcatataaaaatgagtgttttcttggtcagcagatcacaaaaaggacacaaacggtaggttccatctagtcagggggttaggttagggacgccatgtggatgaggcaatcttaattctcggccacaccaagattgtttggtgctcggcgccactaatgcccaatatattacctaaccgagccatccaaatgctacatagaattaatcctcctgtagactgaacccacttg

SEQ ID NO: 43 - DNA, 1665 bp, Triticum aestivum
  source 1..1665: mol_type = genomic DNA
atgagcagccccatggaggaagctcaccatggcatgccgtcgacgacgacggcgttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcatgtgctcaagaccaacttcaacaattaccccaagggggaggtgtacaggtcctacatggacgtgctgctcggcgacggcatcttcaacgccgacggcgagctctggaggaagcagaggaagacggcgagcttcgagttcgcttccaagaacctgagagactttagcacgatcgtgttcagggagtactccctgaagctgcgcagcatcctgagccaggcttgcaaggccggcaaagtcgtggacatgcaggagctgtacatgaggatgacgctggactcgatctgcaaggtggggttcggggtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaagatcaagcacgacatactgtcgcggttcatcgagctgggcgaggccggcggggacgacggcggcagcctgttcggggacgacaagggcctccgcgacgtggtgctcaacttcgtgatcgccgggcgggacaccacggccacgacgctctcctggttcacctacatggccatgacgcacccggccgtggccgagaagctccgccgcgagctggccgccttcgaggcggaccgcgcccgcgaggatggcgtcgcgctggtcccctgcagcgactcagacggcgacggctccgacgaggccttcgccgcccgcgtggcgcagttcgcggggctgctgagctacgacgggctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgcgttccgcaacgcgtcgccgttcaagttcacggcgttccaggcggggccgcggatctgcctcggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 44 - protein, 554 aa, Triticum aestivum
MSSPMEEAHHGMPSTTTAFFPLAGLHKFMAIFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLRSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPAVAEKLRRELAAFEADRAREDGVALVPCSDSDGDGSDEAFAARVAQFAGLLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRAPLA

SEQ ID NO: 45 - DNA, 1920 bp, Triticum aestivum
  source 1..1920: mol_type = genomic DNA
atgagcagccccatggaggaagctcaccttggcatgccgtcgacgacggccttcttcccgctggcagggctccacaagttcatggccgtcttcctcgtgttcctctcgtggatcctggtccactggtggagcctgaggaagcagaaggggccacggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcacgtgctcaagaccaacttcaacaattaccccaaggtgaaacaatcctcgagatgtcagtcaaggttcagtataatcggtactgacagtgttacaaatgtctgaaatctggaattgtgtgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatcctgagccaggcttgcaaggcaggcaaagttgtggacatgcaggtaactgaactctttcccttggtcatatgaacgttgatttcttggacaaaatctcaagattctgacgcgagcgagccaattcaggagctgtacatgaggatgacgctggactcgatctgcaaggtggggttcggggtggagatcggcacgctgtcgccggagctgccggagaacagcttcgcgcaggccttcgacgccgccaacatcatcgtgacgctgcggttcatcgacccgctgtggcgcgtgaagaaattcctgcacgtcggctcggaggcgctgctggagcagagcatcaagctcgtcgacgagttcacctacagcgtcatccgccggcgcaaggccgagatcgtgcaagcccgggccagcggcaagcaggagaaggtgcgtacgtggtcatcgtcattcgtcaagctcccgatcgctggtttgtgcagatgccactgatcactgacacattaactgggcgcgcagatcaagcacgacatactgtcgcggttcatcgagctgggcgaggccggcggcgacgacggcggcagcctgttcggggacgacaagggcctccgcgacgtggtgctcaacttcgtcatcgccgggcgggacacgacggccacgacgctctcctggttcacctacatggccatgacgcacccggccgtggccgagaagctccgccgcgagctggccgccttcgagtccgagcgcgcccgcgaggatggcgtcgctctggtcccctgcagcgacggcgagggctccgacgaggccttcgccgcccgcgtggcgcagttcgcgggactcctgagctacgacgggctcgggaagctggtgtacctccacgcgtgcgtgacggagacgctccgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccagagcggtggatcggcgacgacggcgccttccgcaacgcgtcgccgttcaagttcacggcgttccaggcggggccgcggatctgcctgggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggtgccgctcgcctga

SEQ ID NO: 46 - DNA, 1000 bp, Triticum aestivum
  source 1..1000: mol_type = genomic DNA
gttatcttcatcagatcattgtcaaatttaaagtgttcttcatttattttacatcatccacaactcctacggtagtaccaacagaaaatgatcagcttgaaagttttgaatgcaatgttagggagtactataaagtagagcatatatgtgatagaagtgttctgatctatggaacatatttatttggcagcagtagatgcttccacatatttcaaggtttttaatatttgtgatttttccctgttacaagtttctaataaatatcctcgcaaaaaaaagtttctaataaatagaagatggccactttctcaaaattttcatttgtcccaaagtggtcggatccttcccttgaccctgcgcaagcgggagctacatgcaccgggctgccctttagcttctgctaaaaatttctagcaagtatagaagggcggaactttcaacaaagatatgagaacatcagagcactccttgacaccccttcattccaaatttctcaacttgctctgctttgacttcaaaaactgtctcactgaaagatgcactttgtattggttagtgcgggttcattaaagatcagacggaccataaccatggttccaactgtgaagatgagaccatcacaatgctaactgtcatcaaatgcatcacctacattccctgcaaaataaaaataaaaatgcacgacctacatgtgcagtaaaacagaaggaaaatgcagaatccatgacctagctcagcatcaagcacatacaaacatatctagttatatgcatataaaaatcagtattttcttggtcagcagatcacaaaaaggacacaaacggtaggttccatctagtcagggggttaggttagggacaccatgtggatgaggcaatcttaattctcggccacaccaagattgtttggtgctcggcagcactaatgcccaatatattacctaaccgagccatccaaatgctacatagagttaatcctcctgtagacctgaacccccttc

SEQ ID NO: 47 - DNA, 1656 bp, Triticum aestivum
  source 1..1656: mol_type = genomic DNA
atgagcagccccatggaggaagctcaccttggcatgccgtcgacgacggccttcttcccgctggcagggctccacaagttcatggccgtcttcctcgtgttcctctcgtggatcctggtccactggtggagcctgaggaagcagaaggggccacggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcacgtgctcaagaccaacttcaacaattaccccaagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatcctgagccaggcttgcaaggcaggcaaagttgtggacatgcaggagctgtacatgaggatgacgctggactcgatctgcaaggtggggttcggggtggagatcggcacgctgtcgccggagctgccggagaacagcttcgcgcaggccttcgacgccgccaacatcatcgtgacgctgcggttcatcgacccgctgtggcgcgtgaagaaattcctgcacgtcggctcggaggcgctgctggagcagagcatcaagctcgtcgacgagttcacctacagcgtcatccgccggcgcaaggccgagatcgtgcaagcccgggccagcggcaagcaggagaagatcaagcacgacatactgtcgcggttcatcgagctgggcgaggccggcggcgacgacggcggcagcctgttcggggacgacaagggcctccgcgacgtggtgctcaacttcgtcatcgccgggcgggacacgacggccacgacgctctcctggttcacctacatggccatgacgcacccggccgtggccgagaagctccgccgcgagctggccgccttcgagtccgagcgcgcccgcgaggatggcgtcgctctggtcccctgcagcgacggcgagggctccgacgaggccttcgccgcccgcgtggcgcagttcgcgggactcctgagctacgacgggctcgggaagctggtgtacctccacgcgtgcgtgacggagacgctccgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccagagcggtggatcggcgacgacggcgccttccgcaacgcgtcgccgttcaagttcacggcgttccaggcggggccgcggatctgcctgggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggtgccgctcgcctga

SEQ ID NO: 48 - protein, 551 aa, Triticum aestivum
MSSPMEEAHLGMPSTTAFFPLAGLHKFMAVFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLSSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPAVAEKLRRELAAFESERAREDGVALVPCSDGEGSDEAFAARVAQFAGLLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRVPLA

SEQ ID NO: 49 - DNA, 1920 bp, Triticum aestivum
  source 1..1920: mol_type = genomic DNA
atgagcagccccatggaggaagctcacggcggcatgccgtcgacgacggccttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtgaccgtcgacatgcccttcacctcctacacctacatcgccgacccggtgaacgtcgagcatgtgctcaagaccaacttcaacaattaccccaaggtgaaacaatcctcgagatgtcagtaaaggttcagtataatcggtactgacagtgttacaaatgtctgaaatctgaaattgtatgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatactgagccaggcttgcaaggccggcaaagttgtggacatgcaggtaactgaactcattcccttggtcatctgaacgttgatttcttggacaaaatttcaagattctgacgcgagcgagcgaattcaggagctgtatatgaggatgacgctggactcgatctgcaaagtggggttcggagtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtgcgtggtcatcgtcattcgtcaagctcccggtcgctggtttgtgtagatgccatggatcactgacacactaactgggcgcgcagatcaagcacgacatactgtcgcggttcatcgagctgggcgaggccggcggcgacgacggcggcagtctgttcggggacgacaagggcctccgcgacgtggtgctcaacttcgtgatcgccgggcgggacaccacggccacgacgctgtcctggttcacctacatggccatgacgcacccggacgtggccgagaagctccgccgcgagctggccgccttcgaggcggagcgcgcccgcgaggatggcgtcgctctggtcccctgcggcgacggcgagggctccgacgaggccttcgctgcccgcgtggcgcagttcgcggggttcctgagctacgacggcctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgccttccgcaacgcgtcgccgttcaagttcacggcgttccaggcggggccgcggatttgcctcggcaaggactcggcgtacctgcagatgaagatggcgctggcaatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 50 - DNA, 1000 bp, Triticum aestivum
  source 1..1000: mol_type = genomic DNA
catgaattatcttcatctgacccttgtcaaatttgaagtgatcttcattagttgtacatcatccgcaactgctactgtagtaccaacagaaaacggtcagcttgaaagtttcggaatgcaatgttagggagtactattaagtagagcatatatggatagaagtgttctgatctgcgcaacatatttatttggcagcattagatgcttccacatatgctctgcaaggtttccaatgtttgtgattttttttccctgttacaagtttctactccctccgttcctaaatataagtctttgtagagatttcactatgaaccacatacggatgtatataaatgcattttagaagtagattcactcattttgctccatatgtagtccatagtgaaacctctacaaagacttgtatttaggacggatggagcaataaatagaaggtgatcattttcatcaaaaatttcatttgtttggtcctgttaaaaaattctaattaatatagaagggggaaactttcaacaatattttccatctttgtgattttcaggctttttccatttagggagaacatcagagcaccccttgacaccccttcattccaaatttctcaacttgctctgcttttgacttcaaaaactattggttagtgcgggttcattaaagatcagatggaccataaccatggctccaactgtgaagatgagatcatcacagtgctaattgtcaaaaaaatgcatcacctacatcccccgcaaaagaaaataaaaatgcatcacctacatgtacagtattttcttggtcagcagatcacaaaaaggacacaaacggtaggttccatctagtcagggggttaggttagggacaccatgtggatgaggcaatcttaattctcggccacaccaagattgtttggtgctcggcagcactaatgcccaatatattacctaaccgagccatccaaatgctacatacagttaatcctcctgtagactgaacccccttc

SEQ ID NO: 51 - DNA, 1656 bp, Triticum aestivum
  source 1..1656: mol_type = genomic DNA
atgagcagccccatggaggaagctcacggcggcatgccgtcgacgacggccttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtgaccgtcgacatgcccttcacctcctacacctacatcgccgacccggtgaacgtcgagcatgtgctcaagaccaacttcaacaattaccccaagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatactgagccaggcttgcaaggccggcaaagttgtggacatgcaggagctgtatatgaggatgacgctggactcgatctgcaaagtggggttcggagtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaagatcaagcacgacatactgtcgcggttcatcgagctgggcgaggccggcggcgacgacggcggcagtctgttcggggacgacaagggcctccgcgacgtggtgctcaacttcgtgatcgccgggcgggacaccacggccacgacgctgtcctggttcacctacatggccatgacgcacccggacgtggccgagaagctccgccgcgagctggccgccttcgaggcggagcgcgcccgcgaggatggcgtcgctctggtcccctgcggcgacggcgagggctccgacgaggccttcgctgcccgcgtggcgcagttcgcggggttcctgagctacgacggcctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgccttccgcaacgcgtcgccgttcaagttcacggcgttccaggcggggccgcggatttgcctcggcaaggactcggcgtacctgcagatgaagatggcgctggcaatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 52 - protein, 551 aa, Triticum aestivum
MSSPMEEAHGGMPSTTAFFPLAGLHKFMAIFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLSSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPDVAEKLRRELAAFEAERAREDGVALVPCGDGEGSDEAFAARVAQFAGFLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRAPLA

SEQ ID NO: 53 - DNA, 2026 bp, Zea mays
  source 1..2026: mol_type = genomic DNA
atggaggaagctcacctcacgccggcgacgccatcgccattcttcccactagcagggcctcacaagtacatcgcgctccttctggttgtcctctcatggatcctggtccagaggtggagcctgaggaagcagaaaggcccgagatcatggccagtcatcggcgcaacggtggagcagctgaggaactaccaccggatgcacgactggcttgtcgggtacctgtcacggcacaggacagtgaccgtcgacatgccgttcacttcctacacctacatcgctgacccggtgaatgtcgagcatgtcctcaagactaacttcaccaattaccccaaggtaaatgacctgaactcactgatgttcagtcttcggaaatcagagctgaaagctgaatcgaatgtgcctgaacaccgtgtagggaatcgtgtacagatcctacatggacgtgctcctcggtgacggcatcttcaacgccgacggcgagctgtggaggaagcagaggaagacggcgagtttcgagttcgcctccaagaacctgagggatttcagcgccattgtgttcagagagtactccctgaagctgtcgggtatactgagccaggcatccaaggcaggcaaagttgtggacatgcaggtgagatatcactggtcccttgccattgccaacatgagcatttcaacctgagacacgagagctaccttgccgattcaggaactttacatgaggatgacgctggactccatctgcaaggttgggttcggggtcgagatcggcacgctgtcgccagatctccccgagaacagcttcgcgcaggcgttcgatgccgccaacatcatcatcacgctgcggttcatcgacccgctgtggcgcatcaagaggttcttccacgtcgggtcagaggccctcctagcgcagagcatcaagctcgtggacgagttcacctacagcgtgatccgccggaggaaggccgagatcgtcgaggtccgggccagcggcaaacaggagaaggtacgtgcacatgactgtttcgattcttcagttcatcgtcttggccgggatggacctgatcctgattgattatatatccatgtgacttgtgagggcaaattaaaatgggcagatgaagcacgacatcctgtcacggttcatcgagctgggcgaggccggcgacgacggcggcggcttcggggacgataagagcctccgggacgtggtgctcaacttcgtgatcgccgggcgggacacgacggcgacgacgctgtcgtggttcacgcacatggccatgtcccacccggacgtggccgagaagctgcgccgcgagctgtgcgcgttcgaggcggagcgcgcgcgcgaggagggcgtcacgctcgtgctctgcggcggcgctgacgccgacgacaaggcgttcgccgcccgcgtggcgcagttcgcgggcctcctcacctacgacagcctcggcaagctggtctacctccacgcctgcgtcaccgagacgctccgcctgtaccccgccgtccctcaggtgagcgcgcccgtcacgtcacgacctccggtccgcgatgcaacgcatatgtggctgtccgcagagcacagcatgcagtgagtggacctgaatgcaatgcacatgcacttgcgcgcgcgcaggaccccaaggggatcctggaggacgacgtgctgccggacgggacgaaggtgagggccggcgggatggtgacgtacgtgccctactcgatggggcggatggagtacaactggggccccgacgcggcgagcttccggccggagcggtggatcaacgaggatggcgcgttccgcaacgcgtcgccgttcaagttcacggcgttccaggcggggccgaggatctgcctgggcaaggactcggcgtacctgcagatgaagatggcgctggccatcctcttccgcttctacagcttccggctgctggaggggcacccggtgcagtaccgcatgatgaccatcctctccatggcgcacggcctcaaggtccgcgtctctagggccgtctga

SEQ ID NO: 54 - DNA, 1001 bp, Zea mays
  source 1..1001: mol_type = genomic DNA
ccagggatggcaatggctgtggcaatcggctagaggtggaggacaaggtggtgaggattgggagggcaacctatggcaagttggtgaagaggcacgcaatgagagatctcttcagacttacactggatgccgccaacaaattcaacctttagattttgatactgtcactcctactttattccttggttgggcaacttccaataggctcatgttaatcaatgattagtaattattcagcaaatattcttgtttgtttgacatttataagaagtggggtgagacggattaaatatcatccatgagagctttatcttcatgctctcttgattttggtttcagatcattctttcagtgttcacaataattttctcagtttggtccatgtaatttttgaagtgaggttccttaaatttcattatgcttcctttcttttctagactagcaattgcatgacttttcactttggcttcacaaattgactcacaagaaaacaaattcacttttgggttcacaaattcctcttcaggatgtacttttcacttgaacttcatgtataggaacaaggaatggctcagtttttaaggaacaatgtacagatttcatttcagaactctttctggttggttgagtttcagactttttgtaccaagctgatggatcacaatacttgtttccaaagtctaataacagaaactggcaactcctaattgataatataaaagaataaaatacagtatcagatgtctcattttcttggttggcagatcacaaaaaggaacacaaaggctaagcctcctacttgttcgggagttaggtcagggacaccatatgaatgaaagaaatcttaatttggggtcacaccaagattgtctctctcgaggttgggggatccctaaggttggtagtagcaatacccaatatatcacctaacaaacccaatccatgctacatacatacatagcatccatcacttgtagactggacccttcatcaagagcacc

SEQ ID NO: 55 - DNA, 1632 bp, Zea mays
  source 1..1632: mol_type = genomic DNA
atggaggaagctcacctcacgccggcgacgccatcgccattcttcccactagcagggcctcacaagtacatcgcgctccttctggttgtcctctcatggatcctggtccagaggtggagcctgaggaagcagaaaggcccgagatcatggccagtcatcggcgcaacggtggagcagctgaggaactaccaccggatgcacgactggcttgtcgggtacctgtcacggcacaggacagtgaccgtcgacatgccgttcacttcctacacctacatcgctgacccggtgaatgtcgagcatgtcctcaagactaacttcaccaattaccccaagggaatcgtgtacagatcctacatggacgtgctcctcggtgacggcatcttcaacgccgacggcgagctgtggaggaagcagaggaagacggcgagtttcgagttcgcctccaagaacctgagggatttcagcgccattgtgttcagagagtactccctgaagctgtcgggtatactgagccaggcatccaaggcaggcaaagttgtggacatgcaggaactttacatgaggatgacgctggactccatctgcaaggttgggttcggggtcgagatcggcacgctgtcgccagatctccccgagaacagcttcgcgcaggcgttcgatgccgccaacatcatcatcacgctgcggttcatcgacccgctgtggcgcatcaagaggttcttccacgtcgggtcagaggccctcctagcgcagagcatcaagctcgtggacgagttcacctacagcgtgatccgccggaggaaggccgagatcgtcgaggtccgggccagcggcaaacaggagaagatgaagcacgacatcctgtcacggttcatcgagctgggcgaggccggcgacgacggcggcggcttcggggacgataagagcctccgggacgtggtgctcaacttcgtgatcgccgggcgggacacgacggcgacgacgctgtcgtggttcacgcacatggccatgtcccacccggacgtggccgagaagctgcgccgcgagctgtgcgcgttcgaggcggagcgcgcgcgcgaggagggcgtcacgctcgtgctctgcggcggcgctgacgccgacgacaaggcgttcgccgcccgcgtggcgcagttcgcgggcctcctcacctacgacagcctcggcaagctggtctacctccacgcctgcgtcaccgagacgctccgcctgtaccccgccgtccctcaggaccccaaggggatcctggaggacgacgtgctgccggacgggacgaaggtgagggccggcgggatggtgacgtacgtgccctactcgatggggcggatggagtacaactggggccccgacgcggcgagcttccggccggagcggtggatcaacgaggatggcgcgttccgcaacgcgtcgccgttcaagttcacggcgttccaggcggggccgaggatctgcctgggcaaggactcggcgtacctgcagatgaagatggcgctggccatcctcttccgcttctacagcttccggctgctggaggggcacccggtgcagtaccgcatgatgaccatcctctccatggcgcacggcctcaaggtccgcgtctctagggccgtctga

SEQ ID NO: 56 - protein, 543 aa, Zea mays
MEEAHLTPATPSPFFPLAGPHKYIALLLVVLSWILVQRWSLRKQKGPRSWPVIGATVEQLRNYHRMHDWLVGYLSRHRTVTVDMPFTSYTYIADPVNVEHVLKTNFTNYPKGIVYRSYMDVLLGDGIFNADGELWRKQRKTASFEFASKNLRDFSAIVFREYSLKLSGILSQASKAGKVVDMQELYMRMTLDSICKVGFGVEIGTLSPDLPENSFAQAFDAANIIITLRFIDPLWRIKRFFHVGSEALLAQSIKLVDEFTYSVIRRRKAEIVEVRASGKQEKMKHDILSRFIELGEAGDDGGGFGDDKSLRDVVLNFVIAGRDTTATTLSWFTHMAMSHPDVAEKLRRELCAFEAERAREEGVTLVLCGGADADDKAFAARVAQFAGLLTYDSLGKLVYLHACVTETLRLYPAVPQDPKGILEDDVLPDGTKVRAGGMVTYVPYSMGRMEYNWGPDAASFRPERWINEDGAFRNASPFKFTAFQAGPRICLGKDSAYLQMKMALAILFRFYSFRLLEGHPVQYRMMTILSMAHGLKVRVSRAV

SEQ ID NO: 57 - DNA, 1930 bp, Oryza sativa
  source 1..1930: mol_type = genomic DNA
atgaagagccccatggaggaagctcatgcaatgccagtgacatcattcttcccagtagcaggaatccacaagctcatagctatcttccttgttgtcctctcatggatcttggtccacaagtggagcctgaggaaccagaaagggccaagatcatggccaatcatcggcgcgacagtggagcaactgaagaactaccacaggatgcatgactggcttgtcgagtacttgtcgaaggacaggacggtgaccgtcgacatgcctttcacctcctacacctacattgccgacccggtgaacgtcgagcatgtcctgaagaccaacttcaccaattaccccaaggtaaaagaaccataggatcttcagtgtactgtaaaatgtgccttgcacagtactaacactgacacaaaaaatgtctgaaaatatgcagggtgaagtgtacaggtcttacatggatgtgctgctcggtgatggcatattcaatgccgacggcgagatgtggaggaagcaaaggaagacggcgagcttcgagtttgcctccaagaacttgagagacttcagcactgtggtgttcagggagtactccctgaagctatcaagcattctgagccaagcatgcaaggccggcagagttgtagacatgcaggtaaccaactgaattccttgcctaatacctaaacatttcttgagaaaccaaattgttcagaattctgatgcaagaactaaccaaaattcaggaattgttcatgaggatgacactggactcgatctgcaaggtcgggtttggggttgagatcgggacgctgtcacctgatctcccggagaacagctttgcccaggcattcgacgctgccaacatcatcgtcacgctgcggttcatcgatcctctgtggcgtctgaagaagttcttgcacgtcggatcagaggctctcctcgagcagagcatgaagctggttgatgacttcacctacagcgtgatccgccgccgcaaggctgagatcttgcaggctcgagccagcggcaagcaagagaaggtgatccttcctctcttgctcaaagaatcagtagaactgaactgacatggtaatggtgatgatcagatcggaaaaggttttgtttcttgatatcgttgatttgtaatggcgagcagatcaagcacgacatactgtcgcggttcatcgagctcggggaggccggcggcgacgaggggggcggcagcttcggggacgacaagagcctccgcgacgtggtgctcaacttcgtgatcgccgggcgtgacacgacggcgacgacgctgtcgtggttcacgtacatggcgatgacgcacccggccgtcgccgacaagctccggcgcgagctggccgcgttcgaggatgagcgcgcgcgcgaggagggcgtcgcgctcgccgacgccgccggcgaggcgtcgttcgcggcgcgcgtggcgcagttcgcgtcgctgctgagctacgacgcggtggggaagctggtgtacctgcacgcgtgcgtgacggagacgctccgcctctacccggcggtgccgcaggaccccaaggggatcgtggaggacgacgtgctccccgacggcaccaaggtgcgcgccggcgggatggtgacgtacgtgccctactccatggggaggatggagtacaactggggccccgacgcggcgagcttccggccggagcggtggctcagcggcgacggcggcgcgttccggaacgcgtcgccgttcaagttcaccgcgttccaggccgggccgcggatctgcctcggcaaggactccgcctacctccagatgaagatggcgctcgccatcctcttccgcttctacaccttcgacctcgtcgaggaccaccccgtcaagtaccggatgatgaccatcctctccatggctcacggcctcaaggtccgcgtctccacctccgtctga

SEQ ID NO: 58 - DNA, 946 bp, Oryza sativa
  source 1..946: mol_type = genomic DNA
ggtttcagacttgtttcagtgttgacattggttatttctcaattcattcgagtatttgttgttacatcacaaaggataagttctatagaaaaaatcttccttttcaagtgatgttctttaattttctgtagaattgtgccctgcaatttctcaaatctttgatagatggcttatttgtattgactggaaaagaaattagttgtcaataactagaagctttagagatgcaaagtattggatatatcttggcaatagtattttatattgcttgtttatgtgagaatgttttaactagatggcaactgatttctgggacaaaatcgcttctacaatagcattttatggaactcgtactcgtcgatagcatttcttggatttgggtgtttgtaaatggcatttcttggattttctcttcattaaaatagcctattcagatgaagtagaattcaggtgaagtagaaaccaactactttgggttcacaatttatatttcttttgaggataccccatttcattttagttgtcatcaaagactagacaatatcgacagaaaatggtaagcctggtttcagttggtgacaatttaacagaattcagatggatatggttctgatattagaaggtggcatacctttagtcgctgcaaacgcttcagttatctgaacaaaacaacgaacttggctgagcaggggaaaaaaatactgtagcattcattttgtgtttacatgagtaacgattcttttctaggtggacagatcacaaaaagaaaactaaagctaagatccaactcctaagggtgttaggttagggacaccatatgaatgagacaatcttaattcttggtcacacaaagattgtctcaaggttggtagcatcagtgcccaatatatcacctaactatgccatccaaaatgctacatagcatctcttgtagactgaacccttc

SEQ ID NO: 59 - DNA, 1635 bp, Oryza sativa
  source 1..1635: mol_type = genomic DNA
atgaagagccccatggaggaagctcatgcaatgccagtgacatcattcttcccagtagcaggaatccacaagctcatagctatcttccttgttgtcctctcatggatcttggtccacaagtggagcctgaggaaccagaaagggccaagatcatggccaatcatcggcgcgacagtggagcaactgaagaactaccacaggatgcatgactggcttgtcgagtacttgtcgaaggacaggacggtgaccgtcgacatgcctttcacctcctacacctacattgccgacccggtgaacgtcgagcatgtcctgaagaccaacttcaccaattaccccaagggtgaagtgtacaggtcttacatggatgtgctgctcggtgatggcatattcaatgccgacggcgagatgtggaggaagcaaaggaagacggcgagcttcgagtttgcctccaagaacttgagagacttcagcactgtggtgttcagggagtactccctgaagctatcaagcattctgagccaagcatgcaaggccggcagagttgtagacatgcaggaattgttcatgaggatgacactggactcgatctgcaaggtcgggtttggggttgagatcgggacgctgtcacctgatctcccggagaacagctttgcccaggcattcgacgctgccaacatcatcgtcacgctgcggttcatcgatcctctgtggcgtctgaagaagttcttgcacgtcggatcagaggctctcctcgagcagagcatgaagctggttgatgacttcacctacagcgtgatccgccgccgcaaggctgagatcttgcaggctcgagccagcggcaagcaagagaagatcaagcacgacatactgtcgcggttcatcgagctcggggaggccggcggcgacgaggggggcggcagcttcggggacgacaagagcctccgcgacgtggtgctcaacttcgtgatcgccgggcgtgacacgacggcgacgacgctgtcgtggttcacgtacatggcgatgacgcacccggccgtcgccgacaagctccggcgcgagctggccgcgttcgaggatgagcgcgcgcgcgaggagggcgtcgcgctcgccgacgccgccggcgaggcgtcgttcgcggcgcgcgtggcgcagttcgcgtcgctgctgagctacgacgcggtggggaagctggtgtacctgcacgcgtgcgtgacggagacgctccgcctctacccggcggtgccgcaggaccccaaggggatcgtggaggacgacgtgctccccgacggcaccaaggtgcgcgccggcgggatggtgacgtacgtgccctactccatggggaggatggagtacaactggggccccgacgcggcgagcttccggccggagcggtggctcagcggcgacggcggcgcgttccggaacgcgtcgccgttcaagttcaccgcgttccaggccgggccgcggatctgcctcggcaaggactccgcctacctccagatgaagatggcgctcgccatcctcttccgcttctacaccttcgacctcgtcgaggaccaccccgtcaagtaccggatgatgaccatcctctccatggctcacggcctcaaggtccgcgtctccacctccgtctga

SEQ ID NO: 60 - protein, 544 aa, Oryza sativa
MKSPMEEAHAMPVTSFFPVAGIHKLIAIFLVVLSWILVHKWSLRNQKGPRSWPIIGATVEQLKNYHRMHDWLVEYLSKDRTVTVDMPFTSYTYIADPVNVEHVLKTNFTNYPKGEVYRSYMDVLLGDGIFNADGEMWRKQRKTASFEFASKNLRDFSTVVFREYSLKLSSILSQACKAGRVVDMQELFMRMTLDSICKVGFGVEIGTLSPDLPENSFAQAFDAANIIVTLRFIDPLWRLKKFLHVGSEALLEQSMKLVDDFTYSVIRRRKAEILQARASGKQEKIKHDILSRFIELGEAGGDEGGGSFGDDKSLRDVVLNFVIAGRDTTATTLSWFTYMAMTHPAVADKLRRELAAFEDERAREEGVALADAAGEASFAARVAQFASLLSYDAVGKLVYLHACVTETLRLYPAVPQDPKGIVEDDVLPDGTKVRAGGMVTYVPYSMGRMEYNWGPDAASFRPERWLSGDGGAFRNASPFKFTAFQAGPRICLGKDSAYLQMKMALAILFRFYTFDLVEDHPVKYRMMTILSMAHGLKVRVSTSV

SEQ ID NO: 61 - DNA, 1483 bp, Triticum aestivum
  source 1..1483: mol_type = genomic DNA
atggaagagaagaagccgcggcggcagggagccgcaggacgcgatggcatcgtgcagtacccgcacctcttcatcgcggccctggcgctggccctggtcctcatggaccccttccacctcggcccgctggccgggatcgactaccggccggtgaagcacgagctggcgccgtacagggaggtcatgcagcgctggccgagggacaacggcagccgcctcaggctcggcaggctcgagttcgtcaacgaggtgttcgggccagagtccatcgagttcgaccgccagggccgcgggccctacgccgggctcgccgacggccgcgtcgtgcggtggatgggggacaaggccgggtgggagacgttcgccgtcatgaatcctgactggtattggcttactgcagaaaaaccatagcttacctgtgtgtgtgcaaactaaaatagttttttcggaaaaaaaaaggtcggagaaagtttgtgctaacggagtggagtcgacgacgaagaagcagcacgggaaggagaagtggtgcggccggcctctcgggctgaggttccacagggagaccggcgagctcttcatcgccgacgcgtactatgggctcatggccgttggcgaaagcggcggcgtggcgacctccctggcgagggaggccggcggggacccggtccacttcgccaacgacctcgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatctcacaggaccagcaatttctcctcttctccgagacaacaaactgcaggtgagataaactcaggttttcagtatgatccggctcgagagatccaggaactgatgacgcctttattaatcggctcatgcatgcacactaggatcatgaggtactggctggaaggtccaagagcgggccaggtggaggtgttcgcgaacctgccggggttccccgacaacgtgcgcttgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtggtcgaggtactcgaggaccggggcggcgaggtgatgaagctggtgagcgaggtgagggaggtggaccggaggctgtggatcgggaccgttgcgcacaaccacatcgccacgatcccttatccgttggactag

SEQ ID NO: 62 - DNA, 1000 bp, Triticum aestivum
  source 1..1000: mol_type = genomic DNA
tagtagagatgcttgaagaaataaaccagacttttcttaagcatcagtgcttatttatacatggcagacgcttaattagacgtctctcctataaaaataagcaccggtgcttgagaaaaatccggtttatttttgtaagcacctctcctaagcaccttgcattgtacaaggtcttggagaaataaaccaggctttctttaagtatccgtgcttatttatacaggacagacgcttaattagacgtttctcctgtagaaataggcacaaatgcttcaaaaaaatccgatttgtttttataagcacctagcattgtacgaggccttacgtatttgttgggtgcttaaaaaggaagagaaagaaagaaagaaagcgatctagaaatttaaacactgaagggacccatgtcgtcaccctagggccttccgaaacgtaggaccgaccctacacgcaccgcattacgccaattatctctccctctaatccccttataattacctctataacatctgtcaataactaaatcattatcacgaatgataccgaattcttgactgctcccttgctcttctgcttctttctcctccaaagtttgctcttctctccctgatcctgatcctcaccagatcaggtcatgcatgataattggctcggtatatcctcctggatcactttatgcttgctttttttgagaatccactttatgcttgttgacctgtacatcttgcatcactatccaagcaacgaaggcatgcaaatcccaaattccaaaagcgccatatccccttagctgttctgaaccgaaatacacctactcccaaacgatcacaccgacccatgcaacctccgtgcgtgccgggataatattgtcacgctagctgactcatgcaactcccgtgcatgtcggtatatattttcggggcaaatccattaagaatttaagatcacattgcccgcgcttttttcgtccgcatgcaaactagagccactgccctctacctcc

SEQ ID NO: 63 - DNA, 1242 bp, Triticum aestivum
  source 1..1242: mol_type = genomic DNA
atggaagagaagaagccgcggcggcagggagccgcaggacgcgatggcatcgtgcagtacccgcacctcttcatcgcggccctggcgctggccctggtcctcatggaccccttccacctcggcccgctggccgggatcgactaccggccggtgaagcacgagctggcgccgtacagggaggtcatgcagcgctggccgagggacaacggcagccgcctcaggctcggcaggctcgagttcgtcaacgaggtgttcgggccagagtccatcgagttcgaccgccagggccgcgggccctacgccgggctcgccgacggccgcgtcgtgcggtggatgggggacaaggccgggtgggagacgttcgccgtcatgaatcctgactggtcggagaaagtttgtgctaacggagtggagtcgacgacgaagaagcagcacgggaaggagaagtggtgcggccggcctctcgggctgaggttccacagggagaccggcgagctcttcatcgccgacgcgtactatgggctcatggccgttggcgaaagcggcggcgtggcgacctccctggcgagggaggccggcggggacccggtccacttcgccaacgacctcgacatccacatgaacggctcgatattcttcaccgacacgagcacgagatacagcagaaaggaccatttgaacattttgctggaaggagaaggcacggggaggctgctgagatatgaccgagaaaccggtgccgttcatgtcgtgctcaacgggctggtcttcccaaacggcgtgcagatctcacaggaccagcaatttctcctcttctccgagacaacaaactgcaggatcatgaggtactggctggaaggtccaagagcgggccaggtggaggtgttcgcgaacctgccggggttccccgacaacgtgcgcttgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtggtcgaggtactcgaggaccggggcggcgaggtgatgaagctggtgagcgaggtgagggaggtggaccggaggctgtggatcgggaccgttgcgcacaaccacatcgccacgatcccttatccgttggactag

SEQ ID NO: 64 - protein, 413 aa, Triticum aestivum
MEEKKPRRQGAAGRDGIVQYPHLFIAALALALVLMDPFHLGPLAGIDYRPVKHELAPYREVMQRWPRDNGSRLRLGRLEFVNEVFGPESIEFDRQGRGPYAGLADGRVVRWMGDKAGWETFAVMNPDWSEKVCANGVESTTKKQHGKEKWCGRPLGLRFHRETGELFIADAYYGLMAVGESGGVATSLAREAGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 65 - DNA, 1481 bp, Triticum aestivum
  source 1..1481: mol_type = genomic DNA
atggaagagaagaagccgcggcggcagggagccgcagtacgcgatggcatcgtgcagtacccgcacctcttcatcgcggccctggcgctggccctggtcgtcatggaccccttccacctcggcccgctggctgggatcgactaccggccggtgaagcacgagctggcgccatacagggaggtcatgcagcgctggccgagggacaacggcagccgcctcaggctcggcaggctcgagttcgtcaacgaggtgttcgggccggagtccatcgagttcgacagccagggccgcgggccctacgccgggctcgccgacggccgcgtcgtgcggtggatgggggacaagaccgggtgggagacgttcgccgtcatgaatcctgactggtaattggcttactgcagataaatccatagcttacctgtgtgtttgcaaactaaaatgatttcttgggaaaaaaaaaggtcggagaaagtttgtgctaacggagtggagtcaacgacgaagaagcagcacgggaaggagaagtggtgcggccggcctctcgggctgaggttccacagggagaccggcgagctcttcatcgccgacgcgtactatgggctcatggccgtcggcgaaagcggcggcgtggcgacctccctggcaagggaggtcggcggggacccggtccacttcgccaacgacctcgacatccacatgaacggctcgatattcttcaccgacacgagcacgagatacagcagaaagtgagcggagtactgtcgctgatctccatttttgttcttgagatgttgtgtttgagtgtctgacaccatgactgatcatgcagggatcatttgaacattttgctagaaggagaaggcacggggaggctgctgagatatgaccgagaaaccggtgccgttcatgtcgtgctcaacgggctggtcttcccaaacggcgtgcagatttcacaggaccagcaatttctcctcttctccgagacaaccaactgcaggtgagataaactcaggttttcagtatgatccggctcgagagatccaggaactgatgacggatcatgcatgcacgctaggatcatgaggtactggctggaaggtccaagagcgggccaggtggaggtgttcgcgaacctgccggggttccccgacaacgtgcgcctgaacagcaaggggcagttctgggtggcgatcgactgctgccggacgccgacgcaggaggtgttcgcgaggtggccgtggctgcggaccgcctacttcaagatcccggtgtcgatgaagacgctggggaagatggtgagcatgaagatgtacacgcttctcgcgctcctcgacggcgaggggaacgtggtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 66 - DNA, 1000 bp, Triticum aestivum
  source 1..1000: mol_type = genomic DNA
gggattcaggatcaaggatgccttgtttgttgtcgccattgcgtgtggcaggccagcaacctactgttgtgtcgtgtgcccgtgccggcgacccagctatccggcatccgatgggagcaagttcatccgttctttcaaaaaatcccagctacctattcatccatgatccatcccccaatttcagtgtacacgattagggtttcgatgtataaattttaatttagtctgtcacaagtacgtattcatccatcctaattttgtgtgtcctattcatgcctagggttctcatgtataaatttctaattcttcgtgttctcttttcttcataattttaggatattagcccgccttacaatgttgtctaagacccgtaaaagaaacaatgttctctaagaagcatttgccgggtgcttaaaaaagaagaaaagaaagaaagaaagtgatctgaaaattcaaacactgaaggggcccatgtcgtcgacctagggccttccgaaacgtagaaccaaacctacacgcaccgcattacgccaattatctctccctctaatcctctgacaatttcctttataatgactgtcaataactaaatccttatcacgaatgagaccgaattttgctcttctctccctgtatcctgatcctcaccagatcaggtcatgcatgataattggctcggtatatcctcctggatcactttatgcttgttgacctgtacatcttgcatcactttccaagcaacaaaggcatgcaagtctcaaattccaaaaaggccatatccccttagctgttctgaaccgaaatacacctactcccaaacgatcacaccgacccatgcaacctccgtgcatgtcgggataatcttgtgacgctagctaactcatgcaactcccgtgcatgtcggaatatattttcggggcaaatccattaagaatttaagatcacgttgcccgcgcttttttcgtctgcatgcaaacgagaaccactgccctctgcctcc

SEQ ID NO: 67 - DNA, 1242 bp, Triticum aestivum
  source 1..1242: mol_type = genomic DNA
atggaagagaagaagccgcggcggcagggagccgcagtacgcgatggcatcgtgcagtacccgcacctcttcatcgcggccctggcgctggccctggtcgtcatggaccccttccacctcggcccgctggctgggatcgactaccggccggtgaagcacgagctggcgccatacagggaggtcatgcagcgctggccgagggacaacggcagccgcctcaggctcggcaggctcgagttcgtcaacgaggtgttcgggccggagtccatcgagttcgacagccagggccgcgggccctacgccgggctcgccgacggccgcgtcgtgcggtggatgggggacaagaccgggtgggagacgttcgccgtcatgaatcctgactggtcggagaaagtttgtgctaacggagtggagtcaacgacgaagaagcagcacgggaaggagaagtggtgcggccggcctctcgggctgaggttccacagggagaccggcgagctcttcatcgccgacgcgtactatgggctcatggccgtcggcgaaagcggcggcgtggcgacctccctggcaagggaggtcggcggggacccggtccacttcgccaacgacctcgacatccacatgaacggctcgatattcttcaccgacacgagcacgagatacagcagaaaggatcatttgaacattttgctagaaggagaaggcacggggaggctgctgagatatgaccgagaaaccggtgccgttcatgtcgtgctcaacgggctggtcttcccaaacggcgtgcagatttcacaggaccagcaatttctcctcttctccgagacaaccaactgcaggatcatgaggtactggctggaaggtccaagagcgggccaggtggaggtgttcgcgaacctgccggggttccccgacaacgtgcgcctgaacagcaaggggcagttctgggtggcgatcgactgctgccggacgccgacgcaggaggtgttcgcgaggtggccgtggctgcggaccgcctacttcaagatcccggtgtcgatgaagacgctggggaagatggtgagcatgaagatgtacacgcttctcgcgctcctcgacggcgaggggaacgtggtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 68 - protein, 413 aa, Triticum aestivum
MEEKKPRRQGAAVRDGIVQYPHLFIAALALALVVMDPFHLGPLAGIDYRPVKHELAPYREVMQRWPRDNGSRLRLGRLEFVNEVFGPESIEFDSQGRGPYAGLADGRVVRWMGDKTGWETFAVMNPDWSEKVCANGVESTTKKQHGKEKWCGRPLGLRFHRETGELFIADAYYGLMAVGESGGVATSLAREVGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 69 - DNA, 1471 bp, Triticum aestivum
  source 1..1471: mol_type = genomic DNA
atggaagagaagaaaccgcggcggcagggagccgcagtacgcgatggcatcgtgcagtacccgcacctcttcatcgcggccctggcgctggccctggtcctcatggacccgttccacctcggcccgctggccgggatcgactaccgaccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccttacgccgggctcgccgacggccgcgtcgtgcggtggatgggggacaaggccgggtgggagacgttcgccgtcatgaatcctgactggtactggcttactgcagaaaaacccatagcttacctgtgtgtgtgcagactaaaatagtttctttcataaaaaaaaggtcggagaaagtttgtgctaacggagtggagtcgacgacgaagaagcagcacgggaaggagaagtggtgcggccggcctctcggcctgaggttccacagggagaccggcgagctcttcatcgccgacgcgtactatgggctcatggccgtcggcgaaaggggcggcgtggcgacctccctggcgagggaggccggcggggacccggtccacttcgccaacgaccttgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatatcacaggaccagcaatttctcctcttctccgagacaacaaactgcaggtgagataaactcaggttttcagtatgatccggctcgagagatccaggaactgatgacggctcatgcatgcacactaggatcatgaggtactggctggaaggtccaagagcgggccaggtggaggtgttcgcgaacctgccggggttccccgacaatgtgcgcctgaacagcaaggggcagttctgggtggccatcgactgctgccgtacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 70 - DNA, 1000 bp, Triticum aestivum
  source 1..1000: mol_type = genomic DNA
tctaaggctttgtacaatgcaaggtgcttagtagagatgcttggagaaataaaccatacttttcttaagcatcggtgcttatttatacatggcagacgcttaattagacgcctctcctgtaaaaataagcacccgtgctttagaaaaatccggtttatttttgtaagcacctctcctaagcaccttgcattgtacaaggccttggagaaataaagcaggctttctttaagtatcggtgcttatttgtacaggtcagacgcttaattaggcgtctctcctgtagaaataggcaccgatgcttcaaaaaaaaacccgctctatttttctaagcacataacattgtacaagaccttaagcatttgtcgggtgcttaaaagaaagaaaaagaaagaaagaatgcgatctgaaaatttaaacactgaagggacccatgtcgtcgccctagggccttcctaaacgtaggaccgaccctgcatgcaccgcattacgccaattatctctccctctaatcttcttacaattatctccataacaactgctaataactaaatcattatcacgaatgaggctgaattcttgacttctcccttgctcttctgcttctttctcctccaaagtttgctcttctctccctgtatactgatcctcaccagatcaggtcatgcatgaaaattggctcggtatcctcctggatcactttatgcttgttgacctgtacatcttgcatcactatccaagcaacgaaggcatgcaagtcccaaattccaaaagcgccatatccccttagctgttctgaaccgaaatacacctactcccaaacgatcacaccgacccatgcaacctccgtgcgtgtcgggataatcttgtgacgctagctgactcatgcaactcccgtgcgtgtcggaatatattttcggagcaaatccattaagaatttaagatcacattgcccgcgcttttttcgtctgcatgcaaaacagagccactgccctctacctcc

SEQ ID NO: 71 - DNA, 1242 bp, Triticum aestivum
  source 1..1242: mol_type = genomic DNA
atggaagagaagaaaccgcggcggcagggagccgcagtacgcgatggcatcgtgcagtacccgcacctcttcatcgcggccctggcgctggccctggtcctcatggacccgttccacctcggcccgctggccgggatcgactaccgaccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccttacgccgggctcgccgacggccgcgtcgtgcggtggatgggggacaaggccgggtgggagacgttcgccgtcatgaatcctgactggtcggagaaagtttgtgctaacggagtggagtcgacgacgaagaagcagcacgggaaggagaagtggtgcggccggcctctcggcctgaggttccacagggagaccggcgagctcttcatcgccgacgcgtactatgggctcatggccgtcggcgaaaggggcggcgtggcgacctccctggcgagggaggccggcggggacccggtccacttcgccaacgaccttgacatccacatgaacggctcgatattcttcaccgacacgagcacgagatacagcagaaaggaccatttgaacattttgctggaaggagaaggcacggggaggctgctgagatatgaccgagaaaccggtgccgttcatgtcgtgctcaacgggctggtcttcccaaacggcgtgcagatatcacaggaccagcaatttctcctcttctccgagacaacaaactgcaggatcatgaggtactggctggaaggtccaagagcgggccaggtggaggtgttcgcgaacctgccggggttccccgacaatgtgcgcctgaacagcaaggggcagttctgggtggccatcgactgctgccgtacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 72 - protein, 413 aa, Triticum aestivum
MEEKKPRRQGAAVRDGIVQYPHLFIAALALALVLMDPFHLGPLAGIDYRPVKHELAPYREVMQRWPRDNGSRLRLGRLEFVNEVFGPESIEFDRQGRGPYAGLADGRVVRWMGDKAGWETFAVMNPDWSEKVCANGVESTTKKQHGKEKWCGRPLGLRFHRETGELFIADAYYGLMAVGERGGVATSLAREAGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 73 - DNA, 1551 bp, Zea mays
  source 1..1551: mol_type = genomic DNA
atggagaagaggaacctgcagtggcggcgagggcgtgatggcatcgtgcagtaccctcacctcttcttcgcggccctggcgctggccctcctagtcgcggacccgttcggcctcagtccgctggccgaggtcgactaccggccggtgaagcacgagctcgcgccgtacggggaggtcatgggcagctggcccagagacaatgccagccggctcaggcgcgggaggctggagttcgtcggcgaggtgttcgggccggagtctatcgagttcgatctccagggccgcgggccgtacgccggcctcgccgacggccgcgtcgtgcggtggatgggcgaggaggccgggtgggagacgttcgccgtcatgaatcctgactggtaagtgctcgatatcgctccggcgtccactcgttacatgctataatatagtagtactaagatattttgatctgattttttgcattcttgggagaaacgtcatgcaaaatttgttgtttcttggcaaaggtcagaagaagtctgtgccaatggagtgaactcaacgacgaggaagcagcacgagaaggaggagttctgcggccggccgctcggcctgaggttccacggggagaccggcgagctctacgtcgccgacgcgtactacggtctcatggtcgttggccagagcggcggcgtggcgtcctccgtcgcgagggaagccgacggggaccccatccggttcgcgaacgacctcgatgtgcacaggaatggatccgtattcttcactgacacgagcatgagatacagcagaaagtgagcaaagcgacgtaacaatccggcttctcattttcaaacgcctctgtattctctgctgaaagagtagctcaccagacaagagctgaatttgcagggaccatctgaacatcctgttagaaggagaaggcaccgggaggctgctcaggtatgatccagaaacaagcggtgtccatgtcgtgctcaaggggctggtgttcccaaacggcgtgcagatctcagaggaccatcagtttcttctcttctccgagacaacaaactgcaggtaacaaaaatactatctgacgatgctcatgattctaccgtatccatagtcatgaacacaaaccacacgaatctggccttgaccaggataatgaggtactggctggaaggcccaagagcgggcgaggtagaggtgttcgcgaacctgccgggcttccccgacaacgtgcgctccaacggcaggggccagttctgggtggcgatcgactgctgccggacgccggcgcaggaggtgttcgccaagaggccgtggctccggaccctgtacttcaagttcccgctgtcgctcaaggtgctcacttggaaggccgccaggaggatgcacacggtgctcgcgctcctcgacggcgaagggcgcgtcgtggaggtgctcgaggaccggggccacgaggtgatgaagctggtgagcgaggtgcgggaggtgggccgcaagctgtggatcggaaccgtggcgcacaaccacatcgccaccatcccctaccctttagaggactaa

SEQ ID NO: 74 - DNA, 1001 bp, Zea mays
  source 1..1001: mol_type = genomic DNA
cagaaatgcgaggaccagccatgtctagtgtccactattggcactacccagaacaagatttaaaaaaataaccaaagtaactaatccactcgaaagctatcatgtaatgtttaaagaaacatctattaaaaccacgatcctcttaaaaaacaagcatatttcgaaagagacaaattatgttacagtttacaaacatctaagagcgacaaattatatcgaaaggtaagctatgacgttcagatttttctttttcattcttgttattttgttattgtttttatatacattttcttctcttacaatagagtgattttcttccgattttataaaatgactataaagtcatttttatataagagcacgcatgtcgtagattctcgttcaaaaatctttctgatttttttaagagctagtttggcaaccctgtttctttcaaagaattttgattttttcaaaaaaaattagtttattttctctttataaaatagaaaacacttagaaaaatagagttgccagactagccctagaatgttttcccaataaattacaatcactgtgtataattatttggccagccccataaattatttaaaccgaaactgaaatcgagcgaaaccaaatctgagctatttctctagattagtaaaaagggagagagagaggaagaaatcagttttaagtcattgtccctgagatgtgcggtttggcaacgatagccaccgtaatcatagctcataggtgcctacgtcaggttcggcagctctcgtgtcatctcacatggcatactacatgcttgttcaaccgttcgtcttgttccatcgtccaagccttgcctattctgaaccaagaggatacctactcccaaacaatccatcttactcatgcaacttccatgcaaacacgcacatatgtttcctgaaccaatccattaaagatcacaacagctagcgttctcccgctagcttccctctctcctctgccgatctttttcgtccaccagc

SEQ ID NO: 75 - DNA, 1239 bp, Zea mays
  source 1..1239: mol_type = genomic DNA
atggagaagaggaacctgcagtggcggcgagggcgtgatggcatcgtgcagtaccctcacctcttcttcgcggccctggcgctggccctcctagtcgcggacccgttcggcctcagtccgctggccgaggtcgactaccggccggtgaagcacgagctcgcgccgtacggggaggtcatgggcagctggcccagagacaatgccagccggctcaggcgcgggaggctggagttcgtcggcgaggtgttcgggccggagtctatcgagttcgatctccagggccgcgggccgtacgccggcctcgccgacggccgcgtcgtgcggtggatgggcgaggaggccgggtgggagacgttcgccgtcatgaatcctgactggtcagaagaagtctgtgccaatggagtgaactcaacgacgaggaagcagcacgagaaggaggagttctgcggccggccgctcggcctgaggttccacggggagaccggcgagctctacgtcgccgacgcgtactacggtctcatggtcgttggccagagcggcggcgtggcgtcctccgtcgcgagggaagccgacggggaccccatccggttcgcgaacgacctcgatgtgcacaggaatggatccgtattcttcactgacacgagcatgagatacagcagaaaggaccatctgaacatcctgttagaaggagaaggcaccgggaggctgctcaggtatgatccagaaacaagcggtgtccatgtcgtgctcaaggggctggtgttcccaaacggcgtgcagatctcagaggaccatcagtttcttctcttctccgagacaacaaactgcaggataatgaggtactggctggaaggcccaagagcgggcgaggtagaggtgttcgcgaacctgccgggcttccccgacaacgtgcgctccaacggcaggggccagttctgggtggcgatcgactgctgccggacgccggcgcaggaggtgttcgccaagaggccgtggctccggaccctgtacttcaagttcccgctgtcgctcaaggtgctcacttggaaggccgccaggaggatgcacacggtgctcgcgctcctcgacggcgaagggcgcgtcgtggaggtgctcgaggaccggggccacgaggtgatgaagctggtgagcgaggtgcgggaggtgggccgcaagctgtggatcggaaccgtggcgcacaaccacatcgccaccatcccctaccctttagaggactaa

SEQ ID NO: 76 - protein, 412 aa, Zea mays
MEKRNLQWRRGRDGIVQYPHLFFAALALALLVADPFGLSPLAEVDYRPVKHELAPYGEVMGSWPRDNASRLRRGRLEFVGEVFGPESIEFDLQGRGPYAGLADGRVVRWMGEEAGWETFAVMNPDWSEEVCANGVNSTTRKQHEKEEFCGRPLGLRFHGETGELYVADAYYGLMVVGQSGGVASSVAREADGDPIRFANDLDVHRNGSVFFTDTSMRYSRKDHLNILLEGEGTGRLLRYDPETSGVHVVLKGLVFPNGVQISEDHQFLLFSETTNCRIMRYWLEGPRAGEVEVFANLPGFPDNVRSNGRGQFWVAIDCCRTPAQEVFAKRPWLRTLYFKFPLSLKVLTWKAARRMHTVLALLDGEGRVVEVLEDRGHEVMKLVSEVREVGRKLWIGTVAHNHIATIPYPLED

SEQ ID NO: 77 - DNA, 1561 bp, Oryza sativa
  source 1..1561: mol_type = genomic DNA
atggaagagaagaagcagcagcagcagcgtccacagagagggcgcgatggcatcctgcagtatccgcaccttttcttcgcggcgctggcgctggccctgctcctcaccgacccgttccacctcggcccgctcgccggggtggactaccggccggtgaggcacgagctggcgccgtaccgcgaggtgatggcgcggtggccgcgggacaacggcagccggctcaggcacggcaggctggagttcgtcggagaggtgttcgggccggagtccatcgagttcgaccgccacggccgcggcccctacgccggcctcgccgacggccgcgtcgtgcggtggatgggggaggacgccgggtgggagacgttcgccgtcatgagccctgactggtaacgaacacctcgcctgcattttgctctcgccctccacgaaaacacctctcgtagcagtgtacaattacgtgttcttatattgcaaaaaaaggtcggagaaagtttgtgccaatggggtggagtcgacgacgaagaagcagcacgagatggagcgacggtgcggccggcctctcgggctgaggtttcacggcgagaccggcgagctctacgtcgccgacgcgtactacgggctcatgtccgtcggtccgaacggcggggtggcgacctctctcgcgagagaagtcggcgggagcccggtcaacttcgcgaacgacctcgacatccaccgcaacggctccgtgttcttcaccgacacgagcacgagatacaacagaaagtgtgcagctgcagtatcactctcttcagttgtatcgattctctatttccttctatcgttcaagattttctgattagaatcagttgtgcagggatcatctgaacgttctgctagaaggtgaaggcacagggaggctgctcagatatgacccagaaaccaaagctgcccatgtcgtgctgagcgggctggtcttcccgaatggcgtgcagatttctgacgaccagcagttcctcctcttctccgaaacaacaaactgcaggtgaaatggcacaagctttcacaggttctgaaaatactaaaggttaaacaagattcagaattgattaacattgcacgcatatgctgttctaggataatgcggtactggctggaagggccaagagccgggcaggtggaggtgttcgccgacctgccggggttcccggacaacgtgcgactgagcagcggcggcggcggcggacggttctgggtggcgatcgactgctgcaggacggcggcgcaggaggtgttcgccaagcggccgtggctgcgaacgctctacttcaagctgcccctgacgatgcggacgctggggaagatggtcagcatgcggatgcacaccctcgtcgcgctcctcgacggcgaaggggacgtcgtcgaggtgctcgaggaccggggcggcgaggtgatgcggctggtgagcgaggtgagggaggtggggcgcaagctgtggatcggcaccgtggctcataaccacatcgccacgatcccttacccgttggaagagcagagtagcagcaacgtgcttggtgattga

SEQ ID NO: 78 - DNA, 930 bp, Oryza sativa
  source 1..930: mol_type = genomic DNA
ttatcgaaaccatatataaatgacttttaagataatctccggttttttttaatatgaaacgtcattgatttttaagatatatttgaccattttcaaacctaagaggtggtttattttccctcaagagaacgcgccgttatttttttacgtcacctaaaaaacataattttttggaaaaaaatatatgaaaagacagatgaatatatgattttttttaaaaatacagattcaaactttacttacacaaatagaaacaaaaaacaataaatttgcttatgaatggtcaaattcattatttttatttgtacctatgtaagtcaaatttaaacttcatggtcattgatatatttataaaaaatagttttttagttgaattttaaagtaacaaaaaaaacaaagggacaaatagacttccctataacccaacgcatgctcaaccacagtaaatgttttagctaagacttagagttagttttcttgcaacatcgacacaccattgcctgtgtcttgtgatttttcctgcacgtttagaagggtggcatcgattgaacttctggacacttggagttcttccttcttcgtgatgcaccttttgctttacagcgctagcaatggccatggtcagggccctcagccctggccgggttcggttacatgcatcgtgatatgcttgttgacctgtgcatcttgcaccgtcatcccagcaatgcaaaacatgcaaatccccacttcgaaagcacaagattccttggctattccgaaccaacagaacacctactcccaaacaatcacgctgactcatgcaacctccatgcatcgaatatatattttcgctgcaaaggcattaagaatttaagttaagatcaccgtctccaccaattccgtctttcctctgcgtgcaaattccgtcttccctcgctcctgatctcc

SEQ ID NO: 79 - DNA, 1284 bp, Oryza sativa
  source 1..1284: mol_type = genomic DNA
atggaagagaagaagcagcagcagcagcgtccacagagagggcgcgatggcatcctgcagtatccgcaccttttcttcgcggcgctggcgctggccctgctcctcaccgacccgttccacctcggcccgctcgccggggtggactaccggccggtgaggcacgagctggcgccgtaccgcgaggtgatggcgcggtggccgcgggacaacggcagccggctcaggcacggcaggctggagttcgtcggagaggtgttcgggccggagtccatcgagttcgaccgccacggccgcggcccctacgccggcctcgccgacggccgcgtcgtgcggtggatgggggaggacgccgggtgggagacgttcgccgtcatgagccctgactggtcggagaaagtttgtgccaatggggtggagtcgacgacgaagaagcagcacgagatggagcgacggtgcggccggcctctcgggctgaggtttcacggcgagaccggcgagctctacgtcgccgacgcgtactacgggctcatgtccgtcggtccgaacggcggggtggcgacctctctcgcgagagaagtcggcgggagcccggtcaacttcgcgaacgacctcgacatccaccgcaacggctccgtgttcttcaccgacacgagcacgagatacaacagaaaggatcatctgaacgttctgctagaaggtgaaggcacagggaggctgctcagatatgacccagaaaccaaagctgcccatgtcgtgctgagcgggctggtcttcccgaatggcgtgcagatttctgacgaccagcagttcctcctcttctccgaaacaacaaactgcaggataatgcggtactggctggaagggccaagagccgggcaggtggaggtgttcgccgacctgccggggttcccggacaacgtgcgactgagcagcggcggcggcggcggacggttctgggtggcgatcgactgctgcaggacggcggcgcaggaggtgttcgccaagcggccgtggctgcgaacgctctacttcaagctgcccctgacgatgcggacgctggggaagatggtcagcatgcggatgcacaccctcgtcgcgctcctcgacggcgaaggggacgtcgtcgaggtgctcgaggaccggggcggcgaggtgatgcggctggtgagcgaggtgagggaggtggggcgcaagctgtggatcggcaccgtggctcataaccacatcgccacgatcccttacccgttggaagagcagagtagcagcaacgtgcttggtgattga

SEQ ID NO: 80 - protein, 427 aa, Oryza sativa
MEEKKQQQQRPQRGRDGILQYPHLFFAALALALLLTDPFHLGPLAGVDYRPVRHELAPYREVMARWPRDNGSRLRHGRLEFVGEVFGPESIEFDRHGRGPYAGLADGRVVRWMGEDAGWETFAVMSPDWSEKVCANGVESTTKKQHEMERRCGRPLGLRFHGETGELYVADAYYGLMSVGPNGGVATSLAREVGGSPVNFANDLDIHRNGSVFFTDTSTRYNRKDHLNVLLEGEGTGRLLRYDPETKAAHVVLSGLVFPNGVQISDDQQFLLFSETTNCRIMRYWLEGPRAGQVEVFADLPGFPDNVRLSSGGGGGRFWVAIDCCRTAAQEVFAKRPWLRTLYFKLPLTMRTLGKMVSMRMHTLVALLDGEGDVVEVLEDRGGEVMRLVSEVREVGRKLWIGTVAHNHIATIPYPLEEQSSSNVLGD

SEQ ID NO: 81 - DNA, 23 bp, Triticum aestivum
  source 1..23: mol_type = genomic DNA
gcggccgccgtgctgcgacaagg

SEQ ID NO: 82 - DNA, 23 bp, Triticum aestivum
  source 1..23: mol_type = genomic DNA
gcgtcctcctccgccgtccacgg

SEQ ID NO: 83 - DNA, 23 bp, Triticum aestivum
  source 1..23: mol_type = genomic DNA
gaagaaggggccgtggacggcgg

SEQ ID NO: 84 - DNA, 23 bp, Triticum aestivum
  source 1..23: mol_type = genomic DNA
gaaggggccgtggacggcggagg

SEQ ID NO: 85 - DNA, 23 bp, Triticum aestivum
  source 1..23: mol_type = genomic DNA
ggggccgtggacggcggaggagg

SEQ ID NO: 86 - DNA, 23 bp, Triticum aestivum
  source 1..23: mol_type = genomic DNA
ggtgccatggttggaggtgtagg

SEQ ID NO: 87 - DNA, 23 bp, Triticum aestivum
  source 1..23: mol_type = genomic DNA
gttgccggtgccatggttggagg

SEQ ID NO: 88 - DNA, 23 bp, Triticum aestivum
  source 1..23: mol_type = genomic DNA
gggaacagaggtccagttgccgg

SEQ ID NO: 89 - DNA, 23 bp, Triticum aestivum
  source 1..23: mol_type = genomic DNA
gcacgagaacttcacgcaggagg

SEQ ID NO: 90 - DNA, 23 bp, Triticum aestivum
  source 1..23: mol_type = genomic DNA
gaacttcacgcaggaggaggagg

SEQ ID NO: 91 - DNA, 23 bp, Triticum aestivum
  source 1..23: mol_type = genomic DNA
gcttcccagcatggcatggaggg

SEQ ID NO: 92 - DNA, 23 bp, Triticum aestivum
  source 1..23: mol_type = genomic DNA
gtcaccctccatgccatgctggg

SEQ ID NO: 93 - DNA, 23 bp, Triticum aestivum
  source 1..23: mol_type = genomic DNA
gaaccagctgccggggcggacgg

SEQ ID NO: 94 - DNA, 23 bp, Triticum aestivum
  source 1..23: mol_type = genomic DNA
tgaggtacaccaactacctgagg

SEQ ID NO: 95 - DNA, 23 bp, Triticum aestivum
  source 1..23: mol_type = genomic DNA
gccaggggcggcggcgtccccgg

SEQ ID NO: 96 - DNA, 23 bp, Zea mays
  source 1..23: mol_type = genomic DNA
ggtgccatgggtggaggtgtagg

SEQ ID NO: 97 - DNA, 23 bp, Zea mays
  source 1..23: mol_type = genomic DNA
gttgccggtgccatgggtggagg

SEQ ID NO: 98 - DNA, 23 bp, Zea mays
  source 1..23: mol_type = genomic DNA
ggtccagttgccggtgccatggg

SEQ ID NO: 99 - DNA, 23 bp, Zea mays
  source 1..23: mol_type = genomic DNA
gggcacgttggtccagttgccgg

SEQ ID NO: 100 - DNA, 23 bp, Zea mays
  source 1..23: mol_type = genomic DNA
gcggcaagagctgcaggctgagg

SEQ ID NO: 101 - DNA, 23 bp, Zea mays
  source 1..23: mol_type = genomic DNA
gaagttctcgtgcttcaggttgg

SEQ ID NO: 102 - DNA, 23 bp, Zea mays
  source 1..23: mol_type = genomic DNA
gggtgaagttctcgtgcttcagg

SEQ ID NO: 103 - DNA, 23 bp, Zea mays
  source 1..23: mol_type = genomic DNA
gatgaggtcttcctcctcctggg

SEQ ID NO: 104 - DNA, 23 bp, Zea mays
  source 1..23: mol_type = genomic DNA
gaagcacgagaacttcacccagg

SEQ ID NO: 105 - DNA, 23 bp, Zea mays
  source 1..23: mol_type = genomic DNA
gcacgagaacttcacccaggagg

SEQ ID NO: 106 - DNA, 23 bp, Zea mays
  source 1..23: mol_type = genomic DNA
gcttccgagcatggcgtggaggg

SEQ ID NO: 107 - DNA, 23 bp, Zea mays
  source 1..23: mol_type = genomic DNA
gtcaccctccacgccatgctcgg

SEQ ID NO: 108 - DNA, 23 bp, Zea mays
  source 1..23: mol_type = genomic DNA
gaaccagctgccgggaaggacgg

SEQ ID NO: 109 - DNA, 23 bp, Zea mays
  source 1..23: mol_type = genomic DNA
gcaagaagctgcggcagcgcggg

SEQ ID NO: 110 - DNA, 23 bp, Zea mays
  source 1..23: mol_type = genomic DNA
gcgatggggcggtgggtgagggg

SEQ ID NO: 111 - DNA, 27 bp, Oryza sativa
  source 1..27: mol_type = genomic DNA
gctgcgacaaggcgaacgtgaagaagg

SEQ ID NO: 112 - DNA, 28 bp, Oryza sativa
  source 1..28: mol_type = genomic DNA
gctgcgacaaggcgaacgtgaagaaggg

SEQ ID NO: 113 - DNA, 27 bp, Oryza sativa
  source 1..27: mol_type = genomic DNA
ggcgtacacctccacccacggcaccgg

SEQ ID NO: 114 - DNA, 26 bp, Oryza sativa
  source 1..26: mol_type = genomic DNA
gcgccggcgccgcaggctgctctcgg

SEQ ID NO: 115 - DNA, 26 bp, Oryza sativa
  source 1..26: mol_type = genomic DNA
gcaggctgctctcggccagtatcagg

SEQ ID NO: 116 - DNA, 26 bp, Oryza sativa
  source 1..26: mol_type = genomic DNA
ggctgaggtacaccaactacctgagg

SEQ ID NO: 117 - DNA, 23 bp, Oryza sativa
  source 1..23: mol_type = genomic DNA
gaagcacgagaacttcacgcagg

SEQ ID NO: 118 - DNA, 26 bp, Oryza sativa
  source 1..26: mol_type = genomic DNA
gaagcacgagaacttcacgcaggagg

SEQ ID NO: 119 - DNA, 26 bp, Oryza sativa
  source 1..26: mol_type = genomic DNA
gcacgagaacttcacgcaggaggagg

SEQ ID NO: 120 - DNA, 22 bp, Oryza sativa
  source 1..22: mol_type = genomic DNA
gtcaccctccacgccatgctgg

SEQ ID NO: 121 - DNA, 23 bp, Oryza sativa
  source 1..23: mol_type = genomic DNA
gtcaccctccacgccatgctggg

SEQ ID NO: 122 - DNA, 26 bp, Oryza sativa
  source 1..26: mol_type = genomic DNA
ggctgctctcggccagtatcaggagg

SEQ ID NO: 123 - DNA, 27 bp, Oryza sativa
  source 1..27: mol_type = genomic DNA
ggtcgctgatcgcgaaccagctgccgg

SEQ ID NO: 124 - DNA, 27 bp, Oryza sativa
  source 1..27: mol_type = genomic DNA
gactcgcagcatctgcagctcaactgg

SEQ ID NO: 125 - DNA, 23 bp, Oryza sativa
  source 1..23: mol_type = genomic DNA
gccgacctcatgcagagcatcgg

SEQ ID NO: 126 - DNA, 23 bp, Triticum aestivum
  source 1..23: mol_type = genomic DNA
gttgaggatgcagcagcaggtgg

SEQ ID NO: 127 - DNA, 23 bp, Triticum aestivum
  source 1..23: mol_type = genomic DNA
gaggatgcagcagcaggtggagg

SEQ ID NO: 128 - DNA, 23 bp, Triticum aestivum
  source 1..23: mol_type = genomic DNA
gcagcagcaggtggagggcgtgg

SEQ ID NO: 129 - DNA, 23 bp, Triticum aestivum
  source 1..23: mol_type = genomic DNA
gcagcaggtggagggcgtggtgg

SEQ ID NO: 130 - DNA, 23 bp, Triticum aestivum
  source 1..23: mol_type = genomic DNA
gtggagggcgtggtgggcggcgg

SEQ ID NO: 131 - DNA, 23 bp, Triticum aestivum
  source 1..23: mol_type = genomic DNA
gtggcacatgcagcagccgctgg

SEQ ID NO: 132 - DNA, 23 bp, Triticum aestivum
  source 1..23: mol_type = genomic DNA
gtggtcgtcttcagcgccagcgg

SEQ ID NO: 133 - DNA, 23 bp, Triticum aestivum
  source 1..23: mol_type = genomic DNA
gcaggaggcgcttgacgacgtgg

SEQ ID NO: 134 - DNA, 23 bp, Triticum aestivum
  source 1..23: mol_type = genomic DNA
gtcgtcaagcgcctcctgcttgg

SEQ ID NO: 135 - DNA, 23 bp, Triticum aestivum
  source 1..23: mol_type = genomic DNA
gtggccggcgccgggtccggggg

SEQ ID NO: 136 - DNA, 23 bp, Triticum aestivum
  source 1..23: mol_type = genomic DNA
ggtggccggcgccgggtccgggg

SEQ ID NO: 137 - DNA, 23 bp, Triticum aestivum
  source 1..23: mol_type = genomic DNA
gtggtggccggcgccgggtccgg

SEQ ID NO: 138 - DNA, 23 bp, Triticum aestivum
  source 1..23: mol_type = genomic DNA
ggacgagcgtgccgttgatgtgg

SEQ ID NO: 139 - DNA, 23 bp, Triticum aestivum
  source 1..23: mol_type = genomic DNA
gtgatggcgtgccacatcaacgg

SEQ ID NO: 140 - DNA, 23 bp, Triticum aestivum
  source 1..23: mol_type = genomic DNA
gcgccggcgtccttgaggagcgg

SEQ ID NO: 141 - DNA, 23 bp, Zea mays
  source 1..23: mol_type = genomic DNA
gtcgggagtgagcggcggcgtgg

SEQ ID NO: 142 - DNA, 23 bp, Zea mays
  source 1..23: mol_type = genomic DNA
gggagtgagcggcggcgtggtgg

SEQ ID NO: 143 - DNA, 23 bp, Zea mays
  source 1..23: mol_type = genomic DNA
gcggacgcggcggcggcatccgg

SEQ ID NO: 144 - DNA, 23 bp, Zea mays
  source 1..23: mol_type = genomic DNA
ggacgcggcggcggcatccgggg

SEQ ID NO: 145 - DNA, 23 bp, Zea mays
  source 1..23: mol_type = genomic DNA
gccaccatcgtcgtcgtcgtcgg

SEQ ID NO: 146 - DNA, 23 bp, Zea mays
  source 1..23: mol_type = genomic DNA
gccgacgacgacgacgatggtgg

SEQ ID NO: 147 - DNA, 23 bp, Zea mays
  source 1..23: mol_type = genomic DNA
gacgacgacgatggtggccgcgg

SEQ ID NO: 148 - DNA, 23 bp, Zea mays
  source 1..23: mol_type = genomic DNA
gccagcgccgacgccgagtgcgg

SEQ ID NO: 149 - DNA, 23 bp, Zea mays
  source 1..23: mol_type = genomic DNA
ggtggccgcggcgccgcactcgg

SEQ ID NO: 150 - DNA, 23 bp, Zea mays
  source 1..23: mol_type = genomic DNA
gccgcactcggcgtcggcgctgg

SEQ ID NO: 151 - DNA, 23 bp, Zea mays
  source 1..23: mol_type = genomic DNA
gcactcggcgtcggcgctggcgg

SEQ ID NO: 152 - DNA, 23 bp, Zea mays
  source 1..23: mol_type = genomic DNA
ggcgctggcggtgtacgagcggg

SEQ ID NO: 153 - DNA, 23 bp, Zea mays
  source 1..23: mol_type = genomic DNA
gctggcggtgtacgagcgggtgg

SEQ ID NO: 154 - DNA, 23 bp, Zea mays
  source 1..23: mol_type = genomic DNA
gtacgagcgggtggcgcgcatgg

SEQ ID NO: 155 - DNA, 23 bp, Zea mays
  source 1..23: mol_type = genomic DNA
gagcgggtggcgcgcatggcggg

SEQ ID NO: 156 - DNA, 28 bp, Oryza sativa
  source 1..28: mol_type = genomic DNA
gcgatgatgtcggtgtacgagagggtgg

SEQ ID NO: 157 - DNA, 26 bp, Oryza sativa
  source 1..26: mol_type = genomic DNA
gcggtggtggtgttcagcgcgagcgg

SEQ ID NO: 158 - DNA, 27 bp, Oryza sativa
  source 1..27: mol_type = genomic DNA
gcggtggtggtgttcagcgcgagcggg

SEQ ID NO: 159 - DNA, 26 bp, Oryza sativa
  source 1..26: mol_type = genomic DNA
gctcgccgccgccgccgacatccagg

SEQ ID NO: 160 - DNA, 26 bp, Oryza sativa
  source 1..26: mol_type = genomic DNA
gcgctgtcgcagctcctcccgccggg

SEQ ID NO: 161 - DNA, 27 bp, Oryza sativa
  source 1..27: mol_type = genomic DNA
gttcgtcggcggcaggctcctcggcgg

SEQ ID NO: 162 - DNA, 27 bp, Oryza sativa
  source 1..27: mol_type = genomic DNA
gaaggtgatggcgtgccacatcaatgg

SEQ ID NO: 163 - DNA, 28 bp, Oryza sativa
  source 1..28: mol_type = genomic DNA
ggcaccctcgtccccctcctcaagcagg

SEQ ID NO: 164 - DNA, 23 bp, Oryza sativa
  source 1..23: mol_type = genomic DNA
gtccccctcctcaagcaggccgg

SEQ ID NO: 165 - DNA, 20 bp, Oryza sativa
  source 1..20: mol_type = genomic DNA
gaggggggtcaaaggagtgg

SEQ ID NO: 166 - DNA, 20 bp, Oryza sativa
  source 1..20: mol_type = genomic DNA
ggtgcgaggggggtcaaagg

SEQ ID NO: 167 - DNA, 23 bp, Oryza sativa
  source 1..23: mol_type = genomic DNA
gaattggtgtggtgcgagggggg

SEQ ID NO: 168 - DNA, 21 bp, Oryza sativa
  source 1..21: mol_type = genomic DNA
gaattggtgtggtgcgagggg

SEQ ID NO: 169 - DNA, 21 bp, Oryza sativa
  source 1..21: mol_type = genomic DNA
gtcgagctcgtagacggcggg

SEQ ID NO: 170 - DNA, 20 bp, Oryza sativa
  source 1..20: mol_type = genomic DNA
gtcgagctcgtagacggcgg

SEQ ID NO: 171 - DNA, 23 bp, Triticum aestivum
  source 1..23: mol_type = genomic DNA
ggcagggctccacaagttcatgg

SEQ ID NO: 172 - DNA, 23 bp, Triticum aestivum
  source 1..23: mol_type = genomic DNA
gcttcctcaggctccaccagtgg

SEQ ID NO: 173 - DNA, 23 bp, Triticum aestivum
  source 1..23: mol_type = genomic DNA
gtggagcctgaggaagcagaagg

SEQ ID NO: 174 - DNA, 23 bp, Triticum aestivum
  source 1..23: mol_type = genomic DNA
ggagcctgaggaagcagaagggg

SEQ ID NO: 175 - DNA, 23 bp, Triticum aestivum
  source 1..23: mol_type = genomic DNA
gccggtcatcggcgcgacgctgg

SEQ ID NO: 176 - DNA, 23 bp, Triticum aestivum
  source 1..23: mol_type = genomic DNA
gcgcgacgctggagcagctgagg

SEQ ID NO: 177 - DNA, 23 bp, Triticum aestivum
  source 1..23: mol_type = genomic DNA
gtacctgtccaagcaccggacgg

SEQ ID NO: 178 - DNA, 23 bp, Triticum aestivum
  source 1..23: mol_type = genomic DNA
ggaggtgaagggcatgtcgacgg

SEQ ID NO: 179 - DNA, 23 bp, Triticum aestivum
  source 1..23: mol_type = genomic DNA
gatgtaggtgtaggaggtgaagg

SEQ ID NO: 180 - DNA, 23 bp, Triticum aestivum
  source 1..23: mol_type = genomic DNA
gtcggcgatgtaggtgtaggagg

SEQ ID NO: 181 - DNA, 23 bp, Triticum aestivum
  source 1..23: mol_type = genomic DNA
gttcaccgggtcggcgatgtagg

SEQ ID NO: 182 - DNA, 23 bp, Triticum aestivum
  source 1..23: mol_type = genomic DNA
gccgagcagcacgtccatgtagg

SEQ ID NO: 183 - DNA, 23 bp, Triticum aestivum
  source 1..23: mol_type = genomic DNA
ggaggtgtacaggtcctacatgg

SEQ ID NO: 184 - DNA, 23 bp, Triticum aestivum
  source 1..23: mol_type = genomic DNA
gcgagctctggaggaagcagagg

SEQ ID NO: 185 - DNA, 23 bp, Triticum aestivum
  source 1..23: mol_type = genomic DNA
gcctgcgcgaagctgttctccgg

SEQ ID NO: 186 - DNA, 23 bp, Zea mays
  source 1..23: mol_type = genomic DNA
gcgatggcgtcgccggcgtgagg

SEQ ID NO: 187 - DNA, 23 bp, Zea mays
  source 1..23: mol_type = genomic DNA
ggaggaagctcacctcacgccgg

SEQ ID NO: 188 - DNA, 23 bp, Zea mays
  source 1..23: mol_type = genomic DNA
gctagtgggaagaatggcgatgg

SEQ ID NO: 189 - DNA, 23 bp, Zea mays
  source 1..23: mol_type = genomic DNA
ggccctgctagtgggaagaatgg

SEQ ID NO: 190 - DNA, 23 bp, Zea mays
  source 1..23: mol_type = genomic DNA
ggttgtcctctcatggatcctgg

SEQ ID NO: 191 - DNA, 23 bp, Zea mays
  source 1..23: mol_type = genomic DNA
gcttcctcaggctccacctctgg

SEQ ID NO: 192 - DNA, 23 bp, Zea mays
  source 1..23: mol_type = genomic DNA
gccagtcatcggcgcaacggtgg

SEQ ID NO: 193 - DNA, 23 bp, Zea mays
  source 1..23: mol_type = genomic DNA
gcgcaacggtggagcagctgagg

SEQ ID NO: 194 - DNA, 23 bp, Zea mays
  source 1..23: mol_type = genomic DNA
ggatgcacgactggcttgtcggg

SEQ ID NO: 195 - DNA, 23 bp, Zea mays
  source 1..23: mol_type = genomic DNA
ggcttgtcgggtacctgtcacgg

SEQ ID NO: 196 - DNA, 23 bp, Zea mays
  source 1..23: mol_type = genomic DNA
ggaagtgaacggcatgtcgacgg

SEQ ID NO: 197 - DNA, 23 bp, Zea mays
  source 1..23: mol_type = genomic DNA
ggacatgctcgacattcaccggg

SEQ ID NO: 198 - DNA, 23 bp, Zea mays
  source 1..23: mol_type = genomic DNA
gacggcatcttcaacgccgacgg

SEQ ID NO: 199 - DNA, 23 bp, Zea mays
  source 1..23: mol_type = genomic DNA
gcgagctgtggaggaagcagagg

SEQ ID NO: 200 - DNA, 23 bp, Zea mays
  source 1..23: mol_type = genomic DNA
gtggaggaagcagaggaagacgg

SEQ ID NO: 201 - DNA, 27 bp, Zea mays
  source 1..27: mol_type = genomic DNA
gtactccatccgccccatcgagtaggg

SEQ ID NO: 202 - DNA, 24 bp, Zea mays
  source 1..24: mol_type = genomic DNA
gcacgtacgtcaccatcccgccgg

SEQ ID NO: 203 - DNA, 24 bp, Zea mays
  source 1..24: mol_type = genomic DNA
gacgtacgtgccctactcgatggg

SEQ ID NO: 204 - DNA, 25 bp, Oryza sativa
  source 1..25: mol_type = genomic DNA
gtggattcctgctactgggaagaat

SEQ ID NO: 205 - DNA, 24 bp, Oryza sativa
  source 1..24: mol_type = genomic DNA
ggccttgcatgcttggctcagaat

SEQ ID NO: 206 - DNA, 32 bp, Oryza sativa
  source 1..32: mol_type = genomic DNA
gtcctgaagaccaacttcaccaattaccccaa

SEQ ID NO: 207 - DNA, 31 bp, Oryza sativa
  source 1..31: mol_type = genomic DNA
ggaagacggcgagcttcgagtttgcctccaa

SEQ ID NO: 208 - DNA, 31 bp, Oryza sativa
  source 1..31: mol_type = genomic DNA
gcctctacccggcggtgccgcaggaccccaa

SEQ ID NO: 209 - DNA, 31 bp, Oryza sativa
  source 1..31: mol_type = genomic DNA
gacatcattcttcccagtagcaggaatccac

SEQ ID NO: 210 - DNA, 27 bp, Oryza sativa
  source 1..27: mol_type = genomic DNA
gttgtcctctcatggatcttggtccac

SEQ ID NO: 211 - DNA, 32 bp, Oryza sativa
  source 1..32: mol_type = genomic DNA
gcgcgacagtggagcaactgaagaactaccac

SEQ ID NO: 212 - DNA, 32 bp, Oryza sativa
  source 1..32: mol_type = genomic DNA
ggcgggatggtgacgtacgtgccctactccat

SEQ ID NO: 213 - DNA, 32 bp, Oryza sativa
  source 1..32: mol_type = genomic DNA
gcctacctccagatgaagatggcgctcgccat

SEQ ID NO: 214 - DNA, 32 bp, Oryza sativa
  source 1..32: mol_type = genomic DNA
gaccaccccgtcaagtaccggatgatgaccat

SEQ ID NO: 215 - DNA, 20 bp, Oryza sativa
  source 1..20: mol_type = genomic DNA
acgtacgtgccctactccat

SEQ ID NO: 216 - DNA, 23 bp, Triticum aestivum
  source 1..23: mol_type = genomic DNA
gggccgcgatgaagaggtgcggg

SEQ ID NO: 217 - DNA, 23 bp, Triticum aestivum
  source 1..23: mol_type = genomic DNA
gcgccagggccgcgatgaagagg

SEQ ID NO: 218 - DNA, 23 bp, Triticum aestivum
  source 1..23: mol_type = genomic DNA
gtacccgcacctcttcatcgcgg

SEQ ID NO: 219 - DNA, 23 bp, Triticum aestivum
  source 1..23: mol_type = genomic DNA
gcacctcttcatcgcggccctgg

SEQ ID NO: 220 - DNA, 23 bp, Triticum aestivum
  source 1..23: mol_type = genomic DNA
gccagcgggccgaggtggaaggg

SEQ ID NO: 221 - DNA, 23 bp, Triticum aestivum
  source 1..23: mol_type = genomic DNA
ggcgccagctcgtgcttcaccgg

SEQ ID NO: 222 - DNA, 23 bp, Triticum aestivum
  source 1..23: mol_type = genomic DNA
ggtcatgcagcgctggccgaggg

SEQ ID NO: 223 - DNA, 24 bp, Triticum aestivum
  source 1..24: mol_type = genomic DNA
gcagcgctggccgagggacaacgg

SEQ ID NO: 224 - DNA, 23 bp, Triticum aestivum
  source 1..23: mol_type = genomic DNA
gggacaacggcagccgcctcagg

SEQ ID NO: 225 - DNA, 23 bp, Triticum aestivum
  source 1..23: mol_type = genomic DNA
gcagccgcctcaggctcggcagg

SEQ ID NO: 226 - DNA, 23 bp, Triticum aestivum
  source 1..23: mol_type = genomic DNA
gagttcgtcaacgaggtgttcgg

SEQ ID NO: 227 - DNA, 23 bp, Triticum aestivum
  source 1..23: mol_type = genomic DNA
gacgcggccgtcggcgagcccgg

SEQ ID NO: 228 - DNA, 23 bp, Triticum aestivum
  source 1..23: mol_type = genomic DNA
ggccgcgtcgtgcggtggatggg

SEQ ID NO: 229 - DNA, 23 bp, Triticum aestivum
  source 1..23: mol_type = genomic DNA
gccgcgtcgtgcggtggatgggg

SEQ ID NO: 230 - DNA, 23 bp, Triticum aestivum
  source 1..23: mol_type = genomic DNA
gacggcgaacgtctcccacccgg

SEQ ID NO: 231 - DNA, 23 bp, Triticum aestivum
  source 1..23: mol_type = genomic DNA
gaaagtttgtgctaacggagtgg

SEQ ID NO: 232 - DNA, 23 bp, Zea mays
  source 1..23: mol_type = genomic DNA
ggaacctgcagtggcggcgaggg

SEQ ID NO: 233 - DNA, 23 bp, Zea mays
  source 1..23: mol_type = genomic DNA
gggccgcgaagaagaggtgaggg

SEQ ID NO: 234 - DNA, 23 bp, Zea mays
  source 1..23: mol_type = genomic DNA
gcgccagggccgcgaagaagagg

SEQ ID NO: 235 - DNA, 23 bp, Zea mays
  source 1..23: mol_type = genomic DNA
gtaccctcacctcttcttcgcgg

SEQ ID NO: 236 - DNA, 23 bp, Zea mays
  source 1..23: mol_type = genomic DNA
gactaggagggccagcgccaggg

SEQ ID NO: 237 - DNA, 23 bp, Zea mays
  source 1..23: mol_type = genomic DNA
gaacgggtccgcgactaggaggg

SEQ ID NO: 238 - DNA, 23 bp, Zea mays
  source 1..23: mol_type = genomic DNA
ggccgaacgggtccgcgactagg

SEQ ID NO: 239 - DNA, 23 bp, Zea mays
  source 1..23: mol_type = genomic DNA
ggcgctggccctcctagtcgcgg

SEQ ID NO: 240 - DNA, 23 bp, Zea mays
  source 1..23: mol_type = genomic DNA
gccagcggactgaggccgaacgg

SEQ ID NO: 241 - DNA, 23 bp, Zea mays
  source 1..23: mol_type = genomic DNA
ggcgcgagctcgtgcttcaccgg

SEQ ID NO: 242 - DNA, 23 bp, Zea mays
  source 1..23: mol_type = genomic DNA
ggccgaggtcgactaccggccgg

SEQ ID NO: 243 - DNA, 23 bp, Zea mays
  source 1..23: mol_type = genomic DNA
gcacgagctcgcgccgtacgggg

SEQ ID NO: 244 - DNA, 23 bp, Zea mays
  source 1..23: mol_type = genomic DNA
gcgccgtacggggaggtcatggg

SEQ ID NO: 245 - DNA, 23 bp, Zea mays
  source 1..23: mol_type = genomic DNA
gagccggctggcattgtctctgg

SEQ ID NO: 246 - DNA, 23 bp, Zea mays
  source 1..23: mol_type = genomic DNA
ggcccagagacaatgccagccgg

SEQ ID NO: 247 - DNA, 22 bp, Zea mays
  source 1..22: mol_type = genomic DNA
gctggccgaggtcgactaccgg

SEQ ID NO: 248 - DNA, 23 bp, Zea mays
  source 1..23: mol_type = genomic DNA
ggccgaggtcgactaccggccgg

SEQ ID NO: 249 - DNA, 23 bp, Zea mays
  source 1..23: mol_type = genomic DNA
ggcgcgagctcgtgcttcaccgg

SEQ ID NO: 250 - DNA, 32 bp, Oryza sativa
  source 1..32: mol_type = genomic DNA
gcgtactacgggctcatgtccgtcggtccgaa

SEQ ID NO: 251 - DNA, 33 bp, Oryza sativa
  source 1..33: mol_type = genomic DNA
gggaggctgctcagatatgacccagaaaccaaa

SEQ ID NO: 252 - DNA, 29 bp, Oryza sativa
  source 1..29: mol_type = genomic DNA
gtcgtgctgagcgggctggtcttcccgaa

SEQ ID NO: 253 - DNA, 31 bp, Oryza sativa
  source 1..31: mol_type = genomic DNA
gctggccctgctcctcaccgacccgttccac

SEQ ID NO: 254 - DNA, 29 bp, Oryza sativa
  source 1..29: mol_type = genomic DNA
gtcggagaggtgttcgggccggagtccat

SEQ ID NO: 255 - DNA, 24 bp, Oryza sativa
  source 1..24: mol_type = genomic DNA
gtgcctcaccggccggtagtccac

SEQ ID NO: 256 - DNA, 26 bp, Oryza sativa
  source 1..26: mol_type = genomic DNA
gccggctgccgttgtcccgcggccac

SEQ ID NO: 257 - DNA, 26 bp, Oryza sativa
  source 1..26: mol_type = genomic DNA
gtctcccacccggcgtcctcccccat

SEQ ID NO: 258 - DNA, 25 bp, Oryza sativa
  source 1..25: mol_type = genomic DNA
gctcatgacggcgaacgtctcccac

SEQ ID NO: 259 - DNA, 27 bp, Oryza sativa
  source 1..27: mol_type = genomic DNA
gcttcttcgtcgtcgactccaccccat

SEQ ID NO: 260 - DNA, 27 bp, Oryza sativa
  source 1..27: mol_type = genomic DNA
gacttctctcgcgagagaggtcgccac

SEQ ID NO: 261 - DNA, 24 bp, Oryza sativa
  source 1..24: mol_type = genomic DNA
ggtcgtcagaaatctgcacgccat

SEQ ID NO: 262 - DNA, 30 bp, Oryza sativa
  source 1..30: mol_type = genomic DNA
gaaccccggcaggtcggcgaacacctccac

SEQ ID NO: 263 - DNA, 26 bp, Oryza sativa
  source 1..26: mol_type = genomic DNA
gcttgaagtagagcgttcgcagccac

SEQ ID NO: 264 - DNA, 24 bp, Oryza sativa
  source 1..24: mol_type = genomic DNA
ggtgtgcatccgcatgctgaccat

SEQ ID NO: 265 - DNA, 44 bp, Triticum aestivum
  source 1..44: mol_type = genomic DNA
tacagggaggtcatgcagcgctggccgagggacaacggcagccg

SEQ ID NO: 266 - DNA, 37 bp, Triticum aestivum
  source 1..37: mol_type = genomic DNA
tacagggaggtcatgcagcgagggacaacggcagccg

SEQ ID NO: 267 - DNA, 20 bp, synthetic construct
  misc_feature 1..20: note = DNA version of guideRNA to target DNA target
  source 1..20: mol_type = other DNA
gcggccgccgtgctgcgaca

SEQ ID NO: 268 - DNA, 20 bp, synthetic construct
  misc_feature 1..20: note = DNA version of guideRNA to target DNA target
  source 1..20: mol_type = other DNA
gcgtcctcctccgccgtcca

SEQ ID NO: 269 - DNA, 20 bp, synthetic construct
  misc_feature 1..20: note = DNA version of guideRNA to target DNA target
  source 1..20: mol_type = other DNA
gaagaaggggccgtggacgg

SEQ ID NO: 270 - DNA, 20 bp, synthetic construct
  misc_feature 1..20: note = DNA version of guideRNA to target DNA target
  source 1..20: mol_type = other DNA
gaaggggccgtggacggcgg

SEQ ID NO: 271 - DNA, 20 bp, synthetic construct
  misc_feature 1..20: note = DNA version of guideRNA to target DNA target
  source 1..20: mol_type = other DNA
ggggccgtggacggcggagg

SEQ ID NO: 272 - DNA, 20 bp, synthetic construct
  misc_feature 1..20: note = DNA version of guideRNA to target DNA target
  source 1..20: mol_type = other DNA
ggtgccatggttggaggtgt

SEQ ID NO: 273 - DNA, 20 bp, synthetic construct
  misc_feature 1..20: note = DNA version of guideRNA to target DNA target
  source 1..20: mol_type = other DNA
gttgccggtgccatggttgg

SEQ ID NO: 274 - DNA, 20 bp, synthetic construct
  misc_feature 1..20: note = DNA version of guideRNA to target DNA target
  source 1..20: mol_type = other DNA
gggaacagaggtccagttgc

SEQ ID NO: 275 - DNA, 20 bp, synthetic construct
  misc_feature 1..20: note = DNA version of guideRNA to target DNA target
  source 1..20: mol_type = other DNA
gcacgagaacttcacgcagg

SEQ ID NO: 276 - DNA, 20 bp, synthetic construct
  misc_feature 1..20: note = DNA version of guideRNA to target DNA target
  source 1..20: mol_type = other DNA
gaacttcacgcaggaggagg

SEQ ID NO: 277 - DNA, 20 bp, synthetic construct
  misc_feature 1..20: note = DNA version of guideRNA to target DNA target
  source 1..20: mol_type = other DNA
gcttcccagcatggcatgga

SEQ ID NO: 278 - DNA, 20 bp, synthetic construct
  misc_feature 1..20: note = DNA version of guideRNA to target DNA target
  source 1..20: mol_type = other DNA
gtcaccctccatgccatgct

SEQ ID NO: 279 - DNA, 20 bp, synthetic construct
  misc_feature 1..20: note = DNA version of guideRNA to target DNA target
  source 1..20: mol_type = other DNA
gaaccagctgccggggcgga

SEQ ID NO: 280 - DNA, 20 bp, synthetic construct
  misc_feature 1..20: note = DNA version of guideRNA to target DNA target
  source 1..20: mol_type = other DNA
tgaggtacaccaactacctg

SEQ ID NO: 281 - DNA, 20 bp, synthetic construct
  misc_feature 1..20: note = DNA version of guideRNA to target DNA target
  source 1..20: mol_type = other DNA
gccaggggcggcggcgtccc

SEQ ID NO: 282 - DNA, 20 bp, synthetic construct
  misc_feature 1..20: note = DNA version of guideRNA to target DNA target
  source 1..20: mol_type = other DNA
ggtgccatgggtggaggtgt

SEQ ID NO: 283 - DNA, 20 bp, synthetic construct
  misc_feature 1..20: note = DNA version of guideRNA to target DNA target
  source 1..20: mol_type = other DNA
gttgccggtgccatgggtgg

SEQ ID NO: 284 - DNA, 20 bp, synthetic construct
  misc_feature 1..20: note = DNA version of guideRNA to target DNA target
  source 1..20: mol_type = other DNA
ggtccagttgccggtgccat

SEQ ID NO: 285 - DNA, 20 bp, synthetic construct
  misc_feature 1..20: note = DNA version of guideRNA to target DNA target
  source 1..20: mol_type = other DNA
gggcacgttggtccagttgc

SEQ ID NO: 286 - DNA, 20 bp, synthetic construct
  misc_feature 1..20: note = DNA version of guideRNA to target DNA target
  source 1..20: mol_type = other DNA
gcggcaagagctgcaggctg

SEQ ID NO: 287 - DNA, 20 bp, synthetic construct
  misc_feature 1..20: note = DNA version of guideRNA to target DNA target
  source 1..20: mol_type = other DNA
gaagttctcgtgcttcaggt

SEQ ID NO: 288 - DNA, 20 bp, synthetic construct
  misc_feature 1..20: note = DNA version of guideRNA to target DNA target
  source 1..20: mol_type = other DNA
gggtgaagttctcgtgcttc

SEQ ID NO: 289 - DNA, 20 bp, synthetic construct
  misc_feature 1..20: note = DNA version of guideRNA to target DNA target
  source 1..20: mol_type = other DNA
gatgaggtcttcctcctcct

SEQ ID NO: 290 - DNA, 20 bp, synthetic construct
  misc_feature 1..20: note = DNA version of guideRNA to target DNA target
  source 1..20: mol_type = other DNA
gaagcacgagaacttcaccc

SEQ ID NO: 291 - DNA, 20 bp, synthetic construct
  misc_feature 1..20: note = DNA version of guideRNA to target DNA target
  source 1..20: mol_type = other DNA
gcacgagaacttcacccagg

SEQ ID NO: 292 - DNA, 20 bp, synthetic construct
  misc_feature 1..20: note = DNA version of guideRNA to target DNA target
  source 1..20: mol_type = other DNA
gcttccgagcatggcgtgga

SEQ ID NO: 293 - DNA, 20 bp, synthetic construct
  misc_feature 1..20: note = DNA version of guideRNA to target DNA target
  source 1..20: mol_type = other DNA
gtcaccctccacgccatgct

SEQ ID NO: 294 - DNA, 20 bp, synthetic construct
  misc_feature 1..20: note = DNA version of guideRNA to target DNA target
  source 1..20: mol_type = other DNA
gaaccagctgccgggaagga

SEQ ID NO: 295 - DNA, 20 bp, synthetic construct
  misc_feature 1..20: note = DNA version of guideRNA to target DNA target
  source 1..20: mol_type = other DNA
gcaagaagctgcggcagcgc

SEQ ID NO: 296 - DNA, 20 bp, synthetic construct
  misc_feature 1..20: note = DNA version of guideRNA to target DNA target
  source 1..20: mol_type = other DNA
gcgatggggcggtgggtgag

SEQ ID NO: 297 - DNA, 24 bp, synthetic construct
  misc_feature 1..24: note = DNA version of guideRNA to target DNA target
  source 1..24: mol_type = other DNA
gctgcgacaaggcgaacgtgaaga

SEQ ID NO: 298 - DNA, 25 bp, synthetic construct
  misc_feature 1..25: note = DNA version of guideRNA to target DNA target
  source 1..25: mol_type = other DNA
gctgcgacaaggcgaacgtgaagaa

SEQ ID NO: 299 - DNA, 24 bp, synthetic construct
  misc_feature 1..24: note = DNA version of guideRNA to target DNA target
  source 1..24: mol_type = other DNA
ggcgtacacctccacccacggcac

SEQ ID NO: 300 - DNA, 23 bp, synthetic construct
  misc_feature 1..23: note = DNA version of guideRNA to target DNA target
  source 1..23: mol_type = other DNA
gcgccggcgccgcaggctgctct

SEQ ID NO: 301 - DNA, 23 bp, synthetic construct
  misc_feature 1..23: note = DNA version of guideRNA to target DNA target
  source 1..23: mol_type = other DNA
gcaggctgctctcggccagtatc

SEQ ID NO: 302 - DNA, 23 bp, synthetic construct
  misc_feature 1..23: note = DNA version of guideRNA to target DNA target
  source 1..23: mol_type = other DNA
ggctgaggtacaccaactacctg

SEQ ID NO: 303 - DNA, 20 bp, synthetic construct
  misc_feature 1..20: note = DNA version of guideRNA to target DNA target
  source 1..20: mol_type = other DNA
gaagcacgagaacttcacgc

SEQ ID NO: 304 - DNA, 23 bp, synthetic construct
  misc_feature 1..23: note = DNA version of guideRNA to target DNA target
  source 1..23: mol_type = other DNA
gaagcacgagaacttcacgcagg

SEQ ID NO: 305 - DNA, 23 bp, synthetic construct
  misc_feature 1..23: note = DNA version of guideRNA to target DNA target
  source 1..23: mol_type = other DNA
gcacgagaacttcacgcaggagg

SEQ ID NO: 306 - DNA, 19 bp, synthetic construct
  misc_feature 1..19: note = DNA version of guideRNA to target DNA target
  source 1..19: mol_type = other DNA
gtcaccctccacgccatgc

SEQ ID NO: 307 - DNA, 20 bp, synthetic construct
  misc_feature 1..20: note = DNA version of guideRNA to target DNA target
  source 1..20: mol_type = other DNA
gtcaccctccacgccatgct

SEQ ID NO: 308 - DNA, 23 bp, synthetic construct
  misc_feature 1..23: note = DNA version of guideRNA to target DNA target
  source 1..23: mol_type = other DNA
ggctgctctcggccagtatcagg

SEQ ID NO: 309 - DNA, 24 bp, synthetic construct
  misc_feature 1..24: note = DNA version of guideRNA to target DNA target
  source 1..24: mol_type = other DNA
ggtcgctgatcgcgaaccagctgc

SEQ ID NO: 310 - DNA, 24 bp, synthetic construct
  misc_feature 1..24: note = DNA version of guideRNA to target DNA target
  source 1..24: mol_type = other DNA
gactcgcagcatctgcagctcaac

SEQ ID NO: 311 - DNA, 20 bp, synthetic construct
  misc_feature 1..20: note = DNA version of guideRNA to target DNA target
  source 1..20: mol_type = other DNA
gccgacctcatgcagagcat

SEQ ID NO: 312 - DNA, 20 bp, synthetic construct
  misc_feature 1..20: note = DNA version of guideRNA to target DNA target
  source 1..20: mol_type = other DNA
gttgaggatgcagcagcagg

SEQ ID NO: 313 - DNA, 20 bp, synthetic construct
  misc_feature 1..20: note = DNA version of guideRNA to target DNA target
  source 1..20: mol_type = other DNA
gaggatgcagcagcaggtgg

SEQ ID NO: 314 - DNA, 20 bp, synthetic construct
  misc_feature 1..20: note = DNA version of guideRNA to target DNA target
  source 1..20: mol_type = other DNA
gcagcagcaggtggagggcg

SEQ ID NO: 315 - DNA, 20 bp, synthetic construct
  misc_feature 1..20: note = DNA version of guideRNA to target DNA target
  source 1..20: mol_type = other DNA
gcagcaggtggagggcgtgg

SEQ ID NO: 316 - DNA, 20 bp, synthetic construct
  misc_feature 1..20: note = DNA version of guideRNA to target DNA target
  source 1..20: mol_type = other DNA
gtggagggcgtggtgggcgg

SEQ ID NO: 317 - DNA, 20 bp, synthetic construct
  misc_feature 1..20: note = DNA version of guideRNA to target DNA target
  source 1..20: mol_type = other DNA
gtggcacatgcagcagccgc

SEQ ID NO: 318 - DNA, 20 bp, synthetic construct
  misc_feature 1..20: note = DNA version of guideRNA to target DNA target
  source 1..20: mol_type = other DNA
gtggtcgtcttcagcgccag

SEQ ID NO: 319 - DNA, 20 bp, synthetic construct
  misc_feature 1..20: note = DNA version of guideRNA to target DNA target
  source 1..20: mol_type = other DNA
gcaggaggcgcttgacgacg

SEQ ID NO: 320 - DNA, 20 bp, synthetic construct
  misc_feature 1..20: note = DNA version of guideRNA to target DNA target
  source 1..20: mol_type = other DNA
gtcgtcaagcgcctcctgct

SEQ ID NO: 321 - DNA, 20 bp, synthetic construct
  misc_feature 1..20: note = DNA version of guideRNA to target DNA target
  source 1..20: mol_type = other DNA
gtggccggcgccgggtccgg

SEQ ID NO: 322 - DNA, 20 bp, synthetic construct
  misc_feature 1..20: note = DNA version of guideRNA to target DNA target
  source 1..20: mol_type = other DNA
ggtggccggcgccgggtccg

SEQ ID NO: 323 - DNA, 20 bp, synthetic construct
  misc_feature 1..20: note = DNA version of guideRNA to target DNA target
  source 1..20: mol_type = other DNA
gtggtggccggcgccgggtc

SEQ ID NO: 324 - DNA, 20 bp, synthetic construct
  misc_feature 1..20: note = DNA version of guideRNA to target DNA target
  source 1..20: mol_type = other DNA
ggacgagcgtgccgttgatg

SEQ ID NO: 325 - DNA, 20 bp, synthetic construct
  misc_feature 1..20: note = DNA version of guideRNA to target DNA target
  source 1..20: mol_type = other DNA
gtgatggcgtgccacatcaa

SEQ ID NO: 326 - DNA, 20 bp, synthetic construct
  misc_feature 1..20: note = DNA version of guideRNA to target DNA target
  source 1..20: mol_type = other DNA
gcgccggcgtccttgaggag

SEQ ID NO: 327 - DNA, 20 bp, synthetic construct
  misc_feature 1..20: note = DNA version of guideRNA to target DNA target
  source 1..20: mol_type = other DNA
gtcgggagtgagcggcggcg

SEQ ID NO: 328 - DNA, 20 bp, synthetic construct
  misc_feature 1..20: note = DNA version of guideRNA to target DNA target
  source 1..20: mol_type = other DNA
gggagtgagcggcggcgtgg

SEQ ID NO: 329 - DNA, 20 bp, synthetic construct
  misc_feature 1..20: note = DNA version of guideRNA to target DNA target
  source 1..20: mol_type = other DNA
gcggacgcggcggcggcatc

SEQ ID NO: 330 - DNA, 20 bp, synthetic construct
  misc_feature 1..20: note = DNA version of guideRNA to target DNA target
  source 1..20: mol_type = other DNA
ggacgcggcggcggcatccg

SEQ ID NO: 331 - DNA, 20 bp, synthetic construct
  misc_feature 1..20: note = DNA version of guideRNA to target DNA target
  source 1..20: mol_type = other DNA
gccaccatcgtcgtcgtcgt

SEQ ID NO: 332 - DNA, 20 bp, synthetic construct
  misc_feature 1..20: note = DNA version of guideRNA to target DNA target
  source 1..20: mol_type = other DNA
gccgacgacgacgacgatgg

SEQ ID NO: 333 - DNA, 20 bp, synthetic construct
  misc_feature 1..20: note = DNA version of guideRNA to target DNA target
  source 1..20: mol_type = other DNA
gacgacgacgatggtggccg

SEQ ID NO: 334 - DNA, 20 bp, synthetic construct
  misc_feature 1..20: note = DNA version of guideRNA to target DNA target
  source 1..20: mol_type = other DNA
gccagcgccgacgccgagtg

SEQ ID NO: 335 - DNA, 20 bp, synthetic construct
  misc_feature 1..20: note = DNA version of guideRNA to target DNA target
  source 1..20: mol_type = other DNA
ggtggccgcggcgccgcact

SEQ ID NO: 336 - DNA, 20 bp, synthetic construct
  misc_feature 1..20: note = DNA version of guideRNA to target DNA target
  source 1..20: mol_type = other DNA
gccgcactcggcgtcggcgc

SEQ ID NO: 337 - DNA, 20 bp, synthetic construct
  misc_feature 1..20: note = DNA version of guideRNA to target DNA target
  source 1..20: mol_type = other DNA
gcactcggcgtcggcgctgg

SEQ ID NO: 338 - DNA, 20 bp, synthetic construct
  misc_feature 1..20: note = DNA version of guideRNA to target DNA target
  source 1..20: mol_type = other DNA
ggcgctggcggtgtacgagc

SEQ ID NO: 339 - DNA, 20 bp, synthetic construct
  misc_feature 1..20: note = DNA version of guideRNA to target DNA target
  source 1..20: mol_type = other DNA
gctggcggtgtacgagcggg

SEQ ID NO: 340 - DNA, 20 bp, synthetic construct
  misc_feature 1..20: note = DNA version of guideRNA to target DNA target
  source 1..20: mol_type = other DNA
gtacgagcgggtggcgcgca

SEQ ID NO: 341 - DNA, 20 bp, synthetic construct
  misc_feature 1..20: note = DNA version of guideRNA to target DNA target
  source 1..20: mol_type = other DNA
gagcgggtggcgcgcatggc

SEQ ID NO: 342 - DNA, 25 bp, synthetic construct
  misc_feature 1..25: note = DNA version of guideRNA to target DNA target
  source 1..25: mol_type = other DNA
gcgatgatgtcggtgtacgagaggg

SEQ ID NO: 343 - DNA, 23 bp, synthetic construct
  misc_feature 1..23: note = DNA version of guideRNA to target DNA target
  source 1..23: mol_type = other DNA
gcggtggtggtgttcagcgcgag

SEQ ID NO: 344 - DNA, 24 bp, synthetic construct
  misc_feature 1..24: note = DNA version of guideRNA to target DNA target
  source 1..24: mol_type = other DNA
gcggtggtggtgttcagcgcgagc

SEQ ID NO: 345 - DNA, 23 bp, synthetic construct
  misc_feature 1..23: note = DNA version of guideRNA to target DNA target
  source 1..23: mol_type = other DNA
gctcgccgccgccgccgacatcc

SEQ ID NO: 346 - DNA, 23 bp, synthetic construct
  misc_feature 1..23: note = DNA version of guideRNA to target DNA target
  source 1..23: mol_type = other DNA
gcgctgtcgcagctcctcccgcc

SEQ ID NO: 347 - DNA, 24 bp, synthetic construct
  misc_feature 1..24: note = DNA version of guideRNA to target DNA target
  source 1..24: mol_type = other DNA
gttcgtcggcggcaggctcctcgg

SEQ ID NO: 348 - DNA, 24 bp, synthetic construct
  misc_feature 1..24: note = DNA version of guideRNA to target DNA target
  source 1..24: mol_type = other DNA
gaaggtgatggcgtgccacatcaa

SEQ ID NO: 349 - DNA, 25 bp, synthetic construct
  misc_feature 1..25: note = DNA version of guideRNA to target DNA target
  source 1..25: mol_type = other DNA
ggcaccctcgtccccctcctcaagc

SEQ ID NO: 350 - DNA, 20 bp, synthetic construct
  misc_feature 1..20: note = DNA version of guideRNA to target DNA target
  source 1..20: mol_type = other DNA
gtccccctcctcaagcaggc

SEQ ID NO: 351 - DNA, 17 bp, synthetic construct
  misc_feature 1..17: note = DNA version of guideRNA to target DNA target
  source 1..17: mol_type = other DNA
gaggggggtcaaaggag

SEQ ID NO: 352 - DNA, 17 bp, synthetic construct
  misc_feature 1..17: note = DNA version of guideRNA to target DNA target
  source 1..17: mol_type = other DNA
ggtgcgaggggggtcaa

SEQ ID NO: 353 - DNA, 20 bp, synthetic construct
  misc_feature 1..20: note = DNA version of guideRNA to target DNA target
  source 1..20: mol_type = other DNA
gaattggtgtggtgcgaggg

SEQ ID NO: 354 - DNA, 18 bp, synthetic construct
  misc_feature 1..18: note = DNA version of guideRNA to target DNA target
  source 1..18: mol_type = other DNA
gaattggtgtggtgcgag

SEQ ID NO: 355 - DNA, 18 bp, synthetic construct
  misc_feature 1..18: note = DNA version of guideRNA to target DNA target
  source 1..18: mol_type = other DNA
gtcgagctcgtagacggc

SEQ ID NO: 356 - DNA, 17 bp, synthetic construct
  misc_feature 1..17: note = DNA version of guideRNA to target DNA target
  source 1..17: mol_type = other DNA
gtcgagctcgtagacgg

SEQ ID NO: 357 - DNA, 20 bp, synthetic construct
  misc_feature 1..20: note = DNA version of guideRNA to target DNA target
  source 1..20: mol_type = other DNA
ggcagggctccacaagttca

SEQ ID NO: 358 - DNA, 20 bp, synthetic construct
  misc_feature 1..20: note = DNA version of guideRNA to target DNA target
  source 1..20: mol_type = other DNA
gcttcctcaggctccaccag

SEQ ID NO: 359 - DNA, 20 bp, synthetic construct
  misc_feature 1..20: note = DNA version of guideRNA to target DNA target
  source 1..20: mol_type = other DNA
gtggagcctgaggaagcaga

SEQ ID NO: 360 - DNA, 20 bp, synthetic construct
  misc_feature 1..20: note = DNA version of guideRNA to target DNA target
  source 1..20: mol_type = other DNA
ggagcctgaggaagcagaag

SEQ ID NO: 361 - DNA, 20 bp, synthetic construct
  misc_feature 1..20: note = DNA version of guideRNA to target DNA target
  source 1..20: mol_type = other DNA
gccggtcatcggcgcgacgc

SEQ ID NO: 362 - DNA, 20 bp, synthetic construct
  misc_feature 1..20: note = DNA version of guideRNA to target DNA target
  source 1..20: mol_type = other DNA
gcgcgacgctggagcagctg

SEQ ID NO: 363 - DNA, 20 bp, synthetic construct
  misc_feature 1..20: note = DNA version of guideRNA to target DNA target
  source 1..20: mol_type = other DNA
gtacctgtccaagcaccgga

SEQ ID NO: 364 - DNA, 20 bp, synthetic construct
  misc_feature 1..20: note = DNA version of guideRNA to target DNA target
  source 1..20: mol_type = other DNA
ggaggtgaagggcatgtcga

SEQ ID NO: 365 - DNA, 20 bp, synthetic construct
  misc_feature 1..20: note = DNA version of guideRNA to target DNA target
  source 1..20: mol_type = other DNA
gatgtaggtgtaggaggtga

SEQ ID NO: 366 - DNA, 20 bp, synthetic construct
  misc_feature 1..20: note = DNA version of guideRNA to target DNA target
  source 1..20: mol_type = other DNA
gtcggcgatgtaggtgtagg

SEQ ID NO: 367 - DNA, 20 bp, synthetic construct
  misc_feature 1..20: note = DNA version of guideRNA to target DNA target
  source 1..20: mol_type = other DNA
gttcaccgggtcggcgatgt

SEQ ID NO: 368 - DNA, 20 bp, synthetic construct
  misc_feature 1..20: note = DNA version of guideRNA to target DNA target
  source 1..20: mol_type = other DNA
gccgagcagcacgtccatgt

SEQ ID NO: 369 - DNA, 20 bp, synthetic construct
  misc_feature 1..20: note = DNA version of guideRNA to target DNA target
  source 1..20: mol_type = other DNA
ggaggtgtacaggtcctaca

SEQ ID NO: 370 - DNA, 20 bp, synthetic construct
  misc_feature 1..20: note = DNA version of guideRNA to target DNA target
  source 1..20: mol_type = other DNA
gcgagctctggaggaagcag

SEQ ID NO: 371 - DNA, 20 bp, synthetic construct
  misc_feature 1..20: note = DNA version of guideRNA to target DNA target
  source 1..20: mol_type = other DNA
gcctgcgcgaagctgttctc

SEQ ID NO: 372 - DNA, 20 bp, synthetic construct
  misc_feature 1..20: note = DNA version of guideRNA to target DNA target
  source 1..20: mol_type = other DNA
gcgatggcgtcgccggcgtg

SEQ ID NO: 373 - DNA, 20 bp, synthetic construct
  misc_feature 1..20: note = DNA version of guideRNA to target DNA target
  source 1..20: mol_type = other DNA
ggaggaagctcacctcacgc

SEQ ID NO: 374 - DNA, 20 bp, synthetic construct
  misc_feature 1..20: note = DNA version of guideRNA to target DNA target
  source 1..20: mol_type = other DNA
gctagtgggaagaatggcga

SEQ ID NO: 375 - DNA, 20 bp, synthetic construct
  misc_feature 1..20: note = DNA version of guideRNA to target DNA target
  source 1..20: mol_type = other DNA
ggccctgctagtgggaagaa

SEQ ID NO: 376 - DNA, 20 bp, synthetic construct
  misc_feature 1..20: note = DNA version of guideRNA to target DNA target
  source 1..20: mol_type = other DNA
ggttgtcctctcatggatcc

SEQ ID NO: 377 - DNA, 20 bp, synthetic construct
  misc_feature 1..20: note = DNA version of guideRNA to target DNA target
  source 1..20: mol_type = other DNA
gcttcctcaggctccacctc

SEQ ID NO: 378 - DNA, 20 bp, synthetic construct
  misc_feature 1..20: note = DNA version of guideRNA to target DNA target
  source 1..20: mol_type = other DNA
gccagtcatcggcgcaacgg

SEQ ID NO: 379 - DNA, 20 bp, synthetic construct
  misc_feature 1..20: note = DNA version of guideRNA to target DNA target
  source 1..20: mol_type = other DNA
gcgcaacggtggagcagctg

SEQ ID NO: 380 - DNA, 20 bp, synthetic construct
  misc_feature 1..20: note = DNA version of guideRNA to target DNA target
  source 1..20: mol_type = other DNA
ggatgcacgactggcttgtc

SEQ ID NO: 381 - DNA, 20 bp, synthetic construct
  misc_feature 1..20: note = DNA version of guideRNA to target DNA target
  source 1..20: mol_type = other DNA
ggcttgtcgggtacctgtca

SEQ ID NO: 382 - DNA, 20 bp, synthetic construct
  misc_feature 1..20: note = DNA version of guideRNA to target DNA target
  source 1..20: mol_type = other DNA
ggaagtgaacggcatgtcga

SEQ ID NO: 383 - DNA, 20 bp, synthetic construct
  misc_feature 1..20: note = DNA version of guideRNA to target DNA target
  source 1..20: mol_type = other DNA
ggacatgctcgacattcacc

SEQ ID NO: 384 - DNA, 20 bp, synthetic construct
  misc_feature 1..20: note = DNA version of guideRNA to target DNA target
  source 1..20: mol_type = other DNA
gacggcatcttcaacgccga

SEQ ID NO: 385 - DNA, 20 bp, synthetic construct
  misc_feature 1..20: note = DNA version of guideRNA to target DNA target
  source 1..20: mol_type = other DNA
gcgagctgtggaggaagcag

SEQ ID NO: 386 - DNA, 20 bp, synthetic construct
  misc_feature 1..20: note = DNA version of guideRNA to target DNA target
  source 1..20: mol_type = other DNA
gtggaggaagcagaggaaga

SEQ ID NO: 387 - DNA, 24 bp, synthetic construct
  misc_feature 1..24: note = DNA version of guideRNA to target DNA target
  source 1..24: mol_type = other DNA
gtactccatccgccccatcgagta

SEQ ID NO: 388 - DNA, 21 bp, synthetic construct
  misc_feature 1..21: note = DNA version of guideRNA to target DNA target
  source 1..21: mol_type = other DNA
gcacgtacgtcaccatcccgc

SEQ ID NO: 389 - DNA, 21 bp, synthetic construct
  misc_feature 1..21: note = DNA version of guideRNA to target DNA target
  source 1..21: mol_type = other DNA
gacgtacgtgccctactcgat

SEQ ID NO: 390 - DNA, 18 bp, synthetic construct
  misc_feature 1..18: note = DNA version of guideRNA to target DNA target
  source 1..18: mol_type = other DNA
gtggattcctgctactgg

SEQ ID NO: 391 - DNA, 17 bp, synthetic construct
  misc_feature 1..17: note = DNA version of guideRNA to target DNA target
  source 1..17: mol_type = other DNA
ggccttgcatgcttggc

SEQ ID NO: 392 - DNA, 24 bp, synthetic construct
  misc_feature 1..24: note = DNA version of guideRNA to target DNA target
  source 1..24: mol_type = other DNA
gtcctgaagaccaacttcaccaat

SEQ ID NO: 393 - DNA, 23 bp, synthetic construct
  misc_feature 1..23: note = DNA version of guideRNA to target DNA target
  source 1..23: mol_type = other DNA
ggaagacggcgagcttcgagttt

SEQ ID NO: 394 - DNA, 23 bp, synthetic construct
  misc_feature 1..23: note = DNA version of guideRNA to target DNA target
  source 1..23: mol_type = other DNA
gcctctacccggcggtgccgcag

SEQ ID NO: 395 - DNA, 24 bp, synthetic construct
  misc_feature 1..24: note = DNA version of guideRNA to target DNA target
  source 1..24: mol_type = other DNA
gacatcattcttcccagtagcagg

SEQ ID NO: 396 - DNA, 20 bp, synthetic construct
  misc_feature 1..20: note = DNA version of guideRNA to target DNA target
  source 1..20: mol_type = other DNA
gttgtcctctcatggatctt

SEQ ID NO: 397 - DNA, 25 bp, synthetic construct
  misc_feature 1..25: note = DNA version of guideRNA to target DNA target
  source 1..25: mol_type = other DNA
gcgcgacagtggagcaactgaagaa

SEQ ID NO: 398 - DNA, 25 bp, synthetic construct
  misc_feature 1..25: note = DNA version of guideRNA to target DNA target
  source 1..25: mol_type = other DNA
ggcgggatggtgacgtacgtgccct

SEQ ID NO: 399 - DNA, 25 bp, synthetic construct
  misc_feature 1..25: note = DNA version of guideRNA to target DNA target
  source 1..25: mol_type = other DNA
gcctacctccagatgaagatggcgc

SEQ ID NO: 400 - DNA, 25 bp, synthetic construct
  misc_feature 1..25: note = DNA version of guideRNA to target DNA target
  source 1..25: mol_type = other DNA
gaccaccccgtcaagtaccggatga

SEQ ID NO: 401 - DNA, 20 bp, synthetic construct
  misc_feature 1..20: note = DNA version of guideRNA to target DNA target
  source 1..20: mol_type = other DNA
acgtacgtgccctactccat

SEQ ID NO: 402 - DNA, 20 bp, synthetic construct
  misc_feature 1..20: note = DNA version of guideRNA to target DNA target
  source 1..20: mol_type = other DNA
gggccgcgatgaagaggtgc

SEQ ID NO: 403 - DNA, 20 bp, synthetic construct
  misc_feature 1..20: note = DNA version of guideRNA to target DNA target
  source 1..20: mol_type = other DNA
gcgccagggccgcgatgaag

SEQ ID NO: 404 - DNA, 20 bp, synthetic construct
  misc_feature 1..20: note = DNA version of guideRNA to target DNA target
  source 1..20: mol_type = other DNA
gtacccgcacctcttcatcg

SEQ ID NO: 405 - DNA, 20 bp, synthetic construct
  misc_feature 1..20: note = DNA version of guideRNA to target DNA target
  source 1..20: mol_type = other DNA
gcacctcttcatcgcggccc

SEQ ID NO: 406 - DNA, 20 bp, synthetic construct
  misc_feature 1..20: note = DNA version of guideRNA to target DNA target
  source 1..20: mol_type = other DNA
gccagcgggccgaggtggaa

SEQ ID NO: 407 - DNA, 20 bp, synthetic construct
  misc_feature 1..20: note = DNA version of guideRNA to target DNA target
  source 1..20: mol_type = other DNA
ggcgccagctcgtgcttcac

SEQ ID NO: 408 - DNA, 20 bp, synthetic construct
  misc_feature 1..20: note = DNA version of guideRNA to target DNA target
  source 1..20: mol_type = other DNA
ggtcatgcagcgctggccga

SEQ ID NO: 409 - DNA, 21 bp, synthetic construct
  misc_feature 1..21: note = DNA version of guideRNA to target DNA target
  source 1..21: mol_type = other DNA
gcagcgctggccgagggacaa

SEQ ID NO: 410 - DNA, 20 bp, synthetic construct
  misc_feature 1..20: note = DNA version of guideRNA to target DNA target
  source 1..20: mol_type = other DNA
gggacaacggcagccgcctc

SEQ ID NO: 411 - DNA, 20 bp, synthetic construct
  misc_feature 1..20: note = DNA version of guideRNA to target DNA target
  source 1..20: mol_type = other DNA
gcagccgcctcaggctcggc

SEQ ID NO: 412 - DNA, 20 bp, synthetic construct
  misc_feature 1..20: note = DNA version of guideRNA to target DNA target
  source 1..20: mol_type = other DNA
gagttcgtcaacgaggtgtt

SEQ ID NO: 413 - DNA, 20 bp, synthetic construct
  misc_feature 1..20: note = DNA version of guideRNA to target DNA target
  source 1..20: mol_type = other DNA
gacgcggccgtcggcgagcc

SEQ ID NO: 414 - DNA, 20 bp, synthetic construct
  misc_feature 1..20: note = DNA version of guideRNA to target DNA target
  source 1..20: mol_type = other DNA
ggccgcgtcgtgcggtggat

SEQ ID NO: 415 - DNA, 20 bp, synthetic construct
  misc_feature 1..20: note = DNA version of guideRNA to target DNA target
  source 1..20: mol_type = other DNA
gccgcgtcgtgcggtggatg

SEQ ID NO: 416 - DNA, 20 bp, synthetic construct
  misc_feature 1..20: note = DNA version of guideRNA to target DNA target
  source 1..20: mol_type = other DNA
gacggcgaacgtctcccacc

SEQ ID NO: 417 - DNA, 20 bp, synthetic construct
  misc_feature 1..20: note = DNA version of guideRNA to target DNA target
  source 1..20: mol_type = other DNA
gaaagtttgtgctaacggag

SEQ ID NO: 418 - DNA, 20 bp, synthetic construct
  misc_feature 1..20: note = DNA version of guideRNA to target DNA target
  source 1..20: mol_type = other DNA
ggaacctgcagtggcggcga

SEQ ID NO: 419 - DNA, 20 bp, synthetic construct
  misc_feature 1..20: note = DNA version of guideRNA to target DNA target
  source 1..20: mol_type = other DNA
gggccgcgaagaagaggtga

SEQ ID NO: 420 - DNA, 20 bp, synthetic construct
  misc_feature 1..20: note = DNA version of guideRNA to target DNA target
  source 1..20: mol_type = other DNA
gcgccagggccgcgaagaag

SEQ ID NO: 421 - DNA, 20 bp, synthetic construct
  misc_feature 1..20: note = DNA version of guideRNA to target DNA target
  source 1..20: mol_type = other DNA
gtaccctcacctcttcttcg

SEQ ID NO: 422 - DNA, 20 bp, synthetic construct
  misc_feature 1..20: note = DNA version of guideRNA to target DNA target
  source 1..20: mol_type = other DNA
gactaggagggccagcgcca

SEQ ID NO: 423 - DNA, 20 bp, synthetic construct
  misc_feature 1..20: note = DNA version of guideRNA to target DNA target
  source 1..20: mol_type = other DNA
gaacgggtccgcgactagga

SEQ ID NO: 424 - DNA, 20 bp, synthetic construct
  misc_feature 1..20: note = DNA version of guideRNA to target DNA target
  source 1..20: mol_type = other DNA
ggccgaacgggtccgcgact

SEQ ID NO: 425 - DNA, 20 bp, synthetic construct
  misc_feature 1..20: note = DNA version of guideRNA to target DNA target
  source 1..20: mol_type = other DNA
ggcgctggccctcctagtcg

SEQ ID NO: 426 - DNA, 20 bp, synthetic construct
  misc_feature 1..20: note = DNA version of guideRNA to target DNA target
  source 1..20: mol_type = other DNA
gccagcggactgaggccgaa

SEQ ID NO: 427 - DNA, 20 bp, synthetic construct
  misc_feature 1..20: note = DNA version of guideRNA to target DNA target
  source 1..20: mol_type = other DNA
ggcgcgagctcgtgcttcac

SEQ ID NO: 428 - DNA, 20 bp, synthetic construct
  misc_feature 1..20: note = DNA version of guideRNA to target DNA target
  source 1..20: mol_type = other DNA
ggccgaggtcgactaccggc

SEQ ID NO: 429 - DNA, 20 bp, synthetic construct
  misc_feature 1..20: note = DNA version of guideRNA to target DNA target
  source 1..20: mol_type = other DNA
gcacgagctcgcgccgtacg

SEQ ID NO: 430 - DNA, 20 bp, synthetic construct
  misc_feature 1..20: note = DNA version of guideRNA to target DNA target
  source 1..20: mol_type = other DNA
gcgccgtacggggaggtcat

SEQ ID NO: 431 - DNA, 20 bp, synthetic construct
  misc_feature 1..20: note = DNA version of guideRNA to target DNA target
  source 1..20: mol_type = other DNA
gagccggctggcattgtctc

SEQ ID NO: 432 - DNA, 20 bp, synthetic construct
  misc_feature 1..20: note = DNA version of guideRNA to target DNA target
  source 1..20: mol_type = other DNA
ggcccagagacaatgccagc

SEQ ID NO: 433 - DNA, 19 bp, synthetic construct
  misc_feature 1..19: note = DNA version of guideRNA to target DNA target
  source 1..19: mol_type = other DNA
gctggccgaggtcgactac

SEQ ID NO: 434 - DNA, 20 bp, synthetic construct
  misc_feature 1..20: note = DNA version of guideRNA to target DNA target
  source 1..20: mol_type = other DNA
ggccgaggtcgactaccggc

SEQ ID NO: 435 - DNA, 20 bp, synthetic construct
  misc_feature 1..20: note = DNA version of guideRNA to target DNA target
  source 1..20: mol_type = other DNA
ggcgcgagctcgtgcttcac

SEQ ID NO: 436 - DNA, 24 bp, synthetic construct
  misc_feature 1..24: note = DNA version of guideRNA to target DNA target
  source 1..24: mol_type = other DNA
gcgtactacgggctcatgtccgtc

SEQ ID NO: 437 - DNA, 25 bp, synthetic construct
  misc_feature 1..25: note = DNA version of guideRNA to target DNA target
  source 1..25: mol_type = other DNA
gggaggctgctcagatatgacccag

SEQ ID NO: 438 - DNA, 21 bp, synthetic construct
  misc_feature 1..21: note = DNA version of guideRNA to target DNA target
  source 1..21: mol_type = other DNA
gtcgtgctgagcgggctggtc

SEQ ID NO: 439 - DNA, 24 bp, synthetic construct
  misc_feature 1..24: note = DNA version of guideRNA to target DNA target
  source 1..24: mol_type = other DNA
gctggccctgctcctcaccgaccc

SEQ ID NO: 440 - DNA, 22 bp, synthetic construct
  misc_feature 1..22: note = DNA version of guideRNA to target DNA target
  source 1..22: mol_type = other DNA
gtcggagaggtgttcgggccgg

SEQ ID NO: 441 - DNA, 17 bp, synthetic construct
  misc_feature 1..17: note = DNA version of guideRNA to target DNA target
  source 1..17: mol_type = other DNA
gtgcctcaccggccggt

SEQ ID NO: 442 - DNA, 19 bp, synthetic construct
  misc_feature 1..19: note = DNA version of guideRNA to target DNA target
  source 1..19: mol_type = other DNA
gccggctgccgttgtcccg

SEQ ID NO: 443 - DNA, 19 bp, synthetic construct
  misc_feature 1..19: note = DNA version of guideRNA to target DNA target
  source 1..19: mol_type = other DNA
gtctcccacccggcgtcct

SEQ ID NO: 444 - DNA, 18 bp, synthetic construct
  misc_feature 1..18: note = DNA version of guideRNA to target DNA target
  source 1..18: mol_type = other DNA
gctcatgacggcgaacgt

SEQ ID NO: 445 - DNA, 20 bp, synthetic construct
  misc_feature 1..20: note = DNA version of guideRNA to target DNA target
  source 1..20: mol_type = other DNA
gcttcttcgtcgtcgactcc

SEQ ID NO: 446 - DNA, 20 bp, synthetic construct
  misc_feature 1..20: note = DNA version of guideRNA to target DNA target
  source 1..20: mol_type = other DNA
gacttctctcgcgagagagg

SEQ ID NO: 447 - DNA, 17 bp, synthetic construct
  misc_feature 1..17: note = DNA version of guideRNA to target DNA target
  source 1..17: mol_type = other DNA
ggtcgtcagaaatctgc

SEQ ID NO: 448 - DNA, 23 bp, synthetic construct
  misc_feature 1..23: note = DNA version of guideRNA to target DNA target
  source 1..23: mol_type = other DNA
gaaccccggcaggtcggcgaaca

SEQ ID NO: 449 - DNA, 19 bp, synthetic construct
  misc_feature 1..19: note = DNA version of guideRNA to target DNA target
  source 1..19: mol_type = other DNA
gcttgaagtagagcgttcg

SEQ ID NO: 450 - DNA, 17 bp, synthetic construct
  misc_feature 1..17: note = DNA version of guideRNA to target DNA target
  source 1..17: mol_type = other DNA
ggtgtgcatccgcatgc

SEQ ID NO: 451 - DNA, 1000 bp, Triticum aestivum
  source 1..1000: mol_type = genomic DNA
cacagtcacagacatcattccttcagtatataattgccagataaagaaaaaattcctctcatacacagatacactactacggaaaagtaaaataaaacctgcgagttaaaacatgagatccagccattcaggtaaacacaatgtatcacaattcaaagtacattcaggaaagcaccgccatttctatcgtgaattccatttatccatttgtcctgggcacaaagccctgggcctctgcagcagaaaggatcgaagcgccatcaacacacgctgaacgtagcaaagaataagtctgaatgtaacagtcttgaagagaggaagtataggcatctcggactgcacaatgtggattgcaggtcaagtcagtgcctactaaaacctcactcctcacaagaaagctctcggactgtcttcatattatcttcgttgccccaaagtaacatgtaaacccaattcaaaaggtcttcaaacatcccaaatgccatcatatcacacaagtctttaccgaaaaaatcatatcacacaagtattcagagctcataactgaacaaagagcaaaagaggacgtaatacattggaaaatactactcgatcgatctttagtagccataatcacacttaacatttgtgaatcttaaggaaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggcgcatagtgagatgcagcccacgtgagttcagcaacggtctagcgctgggcttttaggcccgcatgatcgggctttgtcgggtggtcgacgtgttcacgattggggagagcaacgcagcagttcctcttagtttagtcccacctcgcctgtccagcagagttctgaccggtttataaactcgcttgctgcatcagactt

SEQ ID NO: 452 - DNA, 1000 bp, Triticum aestivum
  source 1..1000: mol_type = genomic DNA
accgtttagattattgtcagaaaaaaatatcttacgccctgcggctggtcaatgagcaatgttttctgggtaaatttcaacgggagcttctgtatctcgcctgtggtgaaggagacgctccctgttggctcatactgttttcttatttttagatacaattggctacaacaagtaaaaaactactgtagcatgaagcgaaggcaaaggaaaaaagcaagaccctcggcgtgcgtgctccggtcccatgctcgtcgtcgttagttttacatgcattagaggaaatatggcaaatcgtttcatcgtttcagagaactactaggtatagtcccattttgcggtgtttacggtatctctgatccttttgtggatagttcatagcttcatcccaatgttatctggctcaacctagagaagattcttgtaaagccttgaggatctgtccgttttgtacaattcagttgagaactctgctatgatttgggatatctgatatggttagtaaatcattgatgtggatgatcaatgatttgcagaactgccaactccgtctccgtggatcatataatttataatctctgaagcgaaactctgcgaggacttctcttgtgaaaagctcgaaataaatctacatacacgtttatgttaaaatgtgagaagaatgtagaatgtctagtataaaaacggagtcaaataaccagcgttctcccccacggtgaaaaaagaacctatcacatcggtagaccatttttttataggcaaatcatatcggcagactaacaggaaggctcactattccgtactagcacgatggcccattacggtcttccactattttgggccacaccgagagatccagcctactcgtagcccaccgcacgttccgacggcgcttccgtctggacaagcaacggggggagcagaaggctcgtttgtttagtcccacatcggctagcgaaagggagaacgaccagtttataagccacgcgctgcacacagactc

SEQ ID NO: 453 - DNA, 1000 bp, Triticum aestivum
  source 1..1000: mol_type = genomic DNA
cagaggagccgaaccccaaaaagagaaaaagtgatccctggtggtccggatttggcattgccgactgcccaaacatgcctcgccattttcattctccccttgcatatatgttcgatttgaggttcgcatatagtctagtcatatgcggactatttctgaggtttggttggacgacgtttctcctctctcctattcatgatgccttatgggcaagatatttgtttcactgatattttagttttccccgtttcttaaaacatgcttattctctttagcatggccaacaaatttgtaagagacccaccgagaggtgaccgaccaaaatagacaactaagcatgaaaaaatctcaccggcagaaggaaccgcgcggcgcaagcgcgggtatccctctagtacactactacttgcccatagagccagtggtgtacgagtacgaccgacgatgccttagacttagacttcccagtccgtgcaccggaagcacagaaccgtccacgactccacggacagaattcgctccatcaacacccaccaccacaccagcagtagctttcttcacgtgaaaaacaaccacatatacatgcctgccgtagacgacacccatgtactctaccatatatgtacggtcgagacgatacatcggcacatcgcgcctacgtgcccccaaagcatgaaatcaatcgacatgacacatgaacctcacgtgagcacacctctttttgattttatcttttttgcaaatcctcttttttgatttccggctcggctcctctagttgggcccgaagcagggcatatggcaaatgctccagctcgattccttaagtgggccggctgaggaggatcaagtatacacgaggatacagcccacttggccgtttggtggcatcccgcggtcctggtgcttgctgaacgcagcgaacgaccctggtttagtcccacctcgctcaccggaagaagcatcgatcggtttataagccccgcgcttgcaccctcgct

SEQ ID NO: 454 - DNA, 45 bp, Triticum aestivum
  source 1..45: mol_type = genomic DNA
tacagggaggtcatgcagcgctggcacgagggacaacggcagccg

SEQ ID NO: 455 - DNA, 45 bp, Triticum aestivum; identical to SEQ ID NO: 454
  source 1..45: mol_type = genomic DNA

SEQ ID NO: 456 - DNA, 44 bp, Triticum aestivum; identical to SEQ ID NO: 265
  source 1..44: mol_type = genomic DNA

SEQ ID NO: 457 - DNA, 46 bp, Triticum aestivum
  source 1..46: mol_type = genomic DNA
tacagggaggtcatgcagcgctggcaacgagggacaacggcagccg

SEQ ID NO: 458 - DNA, 45 bp, Triticum aestivum; identical to SEQ ID NO: 454
  source 1..45: mol_type = genomic DNA

SEQ ID NO: 459 - DNA, 45 bp, Triticum aestivum
  source 1..45: mol_type = genomic DNA
tacagggaggtcatgcagcgctggctcgagggacaacggcagccg

(19) United States
(12) Patent Application Publication
(10) Pub. No.: US 2024/0254478 A1
(43) Pub. Date: Aug. 1, 2024

(54) COMPOSITIONS AND METHODS OF MODIFYING A PLANT GENOME TO PRODUCE A MS9, MS22, MS26, OR MS45 MALE-STERILE PLANT

(71) Applicant: PIONEER HI-BRED INTERNATIONAL, INC., JOHNSTON, IA (US)

(72) Inventors: ANDREW MARK CIGAN, MADISON, WI (US); TIM FOX, DES MOINES, IA (US); MANJIT SINGH, DES MOINES, IA (US)

(73) Assignee: PIONEER HI-BRED INTERNATIONAL, INC., JOHNSTON, IA (US)

(21) Appl. No.: 18/437,343

(22) Filed: Feb. 9, 2024

Related U.S. Application Data
(63) Continuation of application No. 17/454,510, filed on Nov. 11, 2021, now Pat. No. 11,926,823.
Application No. 17/454,510 is a continuation of application No. 16/214,897, filed on Dec. 10, 2018, now Pat. No. 11,203,752.
(60) Provisional application No. 62/596,998, filed on Dec. 11, 2017.

(51) Int. Cl. C12N 15/11; C07K 14/415; C12N 9/22; C12N 15/82

(52) U.S. Cl. CPC C12N 15/11; C07K 14/415; C12N 9/22; C12N 15/8213; C12N 15/8289; C12N 2310/20; C12N 2800/80

(57) ABSTRACT

Compositions and methods are provided for genome modification of a nucleotide sequence located in or near a male fertility gene of MS9, MS22, MS26, or MS45 in the genome of a plant cell or plant to produce a male-sterile plant. In some examples, the methods and compositions employ a guide RNA/Cas endonuclease system for modifying or altering target sites located in or near a male fertility gene of MS9, MS22, MS26, or MS45 in the genome of a plant cell, plant or seed to produce a male-sterile plant. Also provided are compositions and methods employing a guide polynucleotide/Cas endonuclease system for genome modification a nucleotide sequence located in or near a male fertility gene of MS9, MS22, MS26, or MS45 in the genome of a plant cell to produce a male-sterile plant. Compositions and methods are also provided for restoring fertility to a Ms9, Ms22, Ms26, or Ms45 nucleotide sequence to a male-sterile Ms9, Ms22, Ms26, or Ms45 plant produced using the methods and compositions described herein.

### FIELD

The disclosure relates to the field of plant molecular biology, in particular, to compositions and methods of modifying a plant's genome to alter the male-fertility of a plant.

### CROSS REFERENCE

This patent application claims the benefit of and priority to U.S. Provisional Application No. 62/596,998, filed Dec. 11, 2017, which is incorporated herein by reference in its entirety.

### Reference to Sequence Listing Submitted Electronically

The official copy of the sequence listing is submitted electronically via EFS-Web as an ASCII formatted sequence listing with a file named 7445USNP_SeqLst_ST25.txt, produced on Dec. 6, 2018, and having a size 227 kilobytes and is filed concurrently with the specification. The sequence listing contained in this ASCII formatted document is part of the specification and is herein incorporated by reference in its entirety.

### BACKGROUND

Development of hybrid plant breeding has made possible considerable advances in quality and quantity of crops produced. Increased yield and combination of desirable characteristics, such as resistance to disease and insects, heat and drought tolerance, along with variations in plant composition are all possible because of hybridization procedures. These procedures frequently rely heavily on providing for a male parent contributing pollen to a female parent to produce the resulting hybrid.

Field crops are bred through techniques that take advantage of the plant's method of pollination. A plant is self-pollinating if pollen from one flower is transferred to the same or another flower of the same plant or a genetically identical plant. A plant is cross-pollinated if the pollen comes from a flower on a different plant.

In certain species, such as Brassica campestris, the plant is normally self-sterile and can only be cross-pollinated. In self-pollinating species, such as soybeans, cotton and wheat, the male and female plants are anatomically juxtaposed. During natural pollination, the male reproductive organs of a given flower pollinate the female reproductive organs of the same flower. Maize has male flowers, located on the tassel, and female flowers, located on the ear, on the same plant and can be bred by both self-pollination and cross-pollination techniques,

The development of hybrids requires the crossing of homozygous inbred parents. A hybrid variety is the cross of two such inbred lines, each of which may have one or more desirable characteristics lacked by the other or which complement the other. The new inbreds are crossed with other inbred lines and the hybrids from these crosses are evaluated to determine which have commercial potential. The hybrid progeny of the first generation is designated F₁. In the development of hybrids only the F₁ hybrid plants are sought. The F₁ hybrid is more vigorous than its inbred parents. This hybrid vigor, or heterosis, can be manifested in many ways, including increased vegetative growth and increased yield.

During hybrid seed production, it is desirable to prevent self-pollination of the female inbred to avoid production and harvesting of female inbred seeds, since they exhibit less vigor than the hybrid seeds. To increase commercial quantities of the resulting hybrid seed, hybrid seed is often obtained using male-sterile female parents. Manual emasculation of the female can be labor intensive and/or impractical, depending on the crop. For example, in wheat, both male flowers and female flowers are located within the same floret on a spike making it challenging to prevent self-pollination. As a result, male-sterile female plants created from either chemical or genetic manipulations are often used in hybrid seed production.

### SUMMARY

Provided herein are methods for producing male-sterile plants. In one embodiment, the method includes introducing a genetic modification into at least one or more endogenous MS9, MS22, MS26 or MS45 polynucleotide sequences in a plant cell, wherein the genetic modification confers male sterility to a plant obtained from the plant cell. In one aspect, the genetic modification is introduced using biotechnology approaches. Accordingly, also provided herein are male-sterile plants that contain a genetic modification in at least one or more endogenous MS9, MS22, MS26 or MS45 polynucleotide sequences. The genetic modification confers male sterility to a plant obtained from the plant cell.

In yet another aspect, the method includes providing to a plant cell a guide RNA and a Cas endonuclease. The RNA and Cas endonuclease are capable of forming a complex that enables the Cas endonuclease to introduce a double strand break at a target site located in or near a male fertility gene of MS9, MS22, MS26, or MS45. The method may additionally include identifying at least one plant cell that has the modification. The modification may be at least one deletion, insertion, or substitution of one or more nucleotides in said MS9, MS22, MS26, or MS45 gene that confers male-sterility to a plant. A male-sterile plant may be obtained from the plant cell.

A male-sterile plant may have at least one altered target site that confers male-sterility to the plant. The target site may originate from a corresponding target site that was recognized and cleaved by a guideRNA/Cas endonuclease system. The target site may be located in or near a male fertility gene of MS9, MS22, MS26, or MS45 and affect the expression level of the MS9, MS22, MS26, or MS45 gene so that the plant is male-sterile.

Also provided herein is a method for producing a male sterile plant that includes obtaining or providing a first plant comprising at least one Cas endonuclease capable of introducing a double strand break at a genomic target site located in a male fertility gene locus of MS9, MS22, MS26 or MS45 in the plant genome and a second plant comprising a guide RNA that is capable of forming a complex with the Cas endonuclease. In some aspects, the first and second plants may be crossed and the progeny evaluated for those that have an altered target site. Male-sterile progeny plants may be selected. Accordingly, also included herein are male-sterile progeny plants produced by any of the methods disclosed herein. The progeny plant may include at least one altered target site that originated from a corresponding target site that was recognized and cleaved by a guideRNA/Cas endonuclease system. The altered target site may be located in or near a male fertility gene of MS9, MS22, MS26, or MS45 and affects the expression level of the MS9, MS22, MS26, or MS45 gene so that the plant is male-sterile.

A method of modifying the male-fertility of a plant that includes introducing at least one guide RNA, at least one polynucleotide modification template and at least one Cas endonuclease into a plant cell is provided herein. The Cas endonuclease may introduce a double-strand break at a target site located in or near a MS9, MS22, MS26, or MS45 gene in the genome of the plant cell. The polynucleotide modification template includes at least one nucleotide modification of a nucleotide sequence at the target site, and the modification modifies the expression level of the MS9, MS22, MS26 or MS45 gene. A male-sterile plant may be obtained from the plant cell.

Also provided herein are methods for restoring male fertility in a male-sterile plant. A male sterile plant produced by any of the methods disclosed herein and having one or more endogenous MS9, MS22, MS26 or MS45 genes with a genetic modification that confers male-sterility to the plant may have fertility restored by introducing one or more polynucleotide sequences that encode a MS9, MS22, MS26 or MS45 polypeptide.

Also provided herein are isolated nucleic acids that impact male fertility of a plant. In some aspects, the nucleic acid has a polynucleotide sequence selected from the group consisting of: (a) a polynucleotide comprising the sequence set forth in SEQ ID NO1, 3, 5, 7, 9, 11, 13, 15, 17, or 19; (b) a polynucleotide having at least 85%, 90% or 95% sequence identity to SEQ ID NO1, 3, 5, 7, 9, 11, 13, 15, 17, or 19; (c) a polynucleotide that encodes a polypeptide having at least 85%, 90% or 95% sequence identity to SEQ ID NO: 4, 8, 12, 16, or 20; (d) a polynucleotide that encodes a polypeptide of SEQ ID NO: 4, 8, 12, 16, or 20; (e) a polynucleotide sequence comprising at least 500 consecutive nucleotides of SEQ ID NO: 1, 3, 5, 7, 9, 11, 13, 15, 17, or 19, wherein said polynucleotide encodes a polypeptide that impacts male fertility; and (f) a polynucleotide sequence which hybridizes to the full length of SEQ ID NO: 1, 3, 5, 7, 9, 11, 13, 15, 17, or 19 under highly stringent conditions of a wash of 0.1 SSC, 0.1% (w/v) SDS at 65 degrees Celsius. In some aspects, the nucleic acid is in an expression vector.

Also provided herein is an isolated polypeptide that impacts the male fertility of a plant. The polypeptide has an amino acid sequence selected from the group consisting of: (a) an amino acid sequence that has at least 85%, 90% or 95% sequence identity to the amino acid sequence set forth in SEQ ID NO: 4, 8, 12, 16, or 20, wherein said polypeptide impacts the male fertility of the plant, (b) an amino acid sequence comprising the amino acid sequence set forth in SEQ ID NO: 4, 8, 12, 16, or 20; (c) an amino acid sequence comprising at least 100 contiguous amino acids of the amino acid sequence set forth in SEQ ID NO: 4, 8, 12, 16, or 20, (d) an amino acid sequence encoded by a polynucleotide that has at least 85%, 90% or 95% sequence identity to SEQ ID NO: 1, 3, 5, 7, 9, 11, 13, 15, 17, or 19; and (e) an amino acid sequence encoded by a polynucleotide of SEQ ID NO: 1, 3, 5, 7, 9, 11, 13, 15, 17, or 19; and (f) a polynucleotide sequence which hybridizes to the full length of SEQ ID NO:1, 3, 5, 7, 9, 11, 13, 15, 17, or 19 under highly stringent conditions of a wash of 0.1 SSC, 0.1% (w/v) SDS at 65 degrees Celsius. Also provided herein are plant cells or plants having the nucleic acid and/or expressing the polypeptide.

In another aspect, disclosed herein is an isolated regulatory region driving male-tissue-preferred expression that includes the sequence of SEQ ID NO: 2, 6, 10, 14, and 18 and functional fragments thereof. Also disclosed herein are plant cells comprising the regulatory region. The regulatory region may be operably linked to a heterologous coding sequence. In some aspects, the regulatory region is included in a DNA construct to drive expression of a sequence of interest, for example, a heterologous polynucleotide. The regulatory region may be used to express a gene product in male tissue of a plant. In one aspect, the method includes introducing into the plant a polynucleotide comprising a polynucleotide sequence selected from the group consisting of SEQ ID NO:2, 6, 10, 14, and 18, and functional fragments thereof. The polynucleotide sequence may confer male-tissue-preferred expression of an operably linked sequence.

Also provided herein are methods of expressing a polynucleotide in a plant cell. In one aspect, the method includes introducing into a plant cell a polynucleotide of SEQ ID NO:451, 452 or 453, and functional fragments and variants thereof. The polynucleotide sequence may be used to drive expression of an operably linked heterologous polynucleotide sequence.

### BRIEF DESCRIPTION OF THE FIGURES

FIG. 1A-FIG. 1E: Flower and anther phenotypes from wild-type and TaMs45 mutant wheat plants. FIG. 1A: Male fertile and male sterile wheat heads (white arrows mark extruded anthers). Insert at the bottom of the panel shows anthers from wild-type (left) and Tams45abd plants (right) at late uninucleate pollen stage. FIG. 1B-FIG. 1E: Cross sections of anthers from the following plants; wild-type (FIG. 1B), Tams45abd (FIG. 1C), Tams45 A/a,B/b,d/d (FIG. 1D) and Tams45a,b,d containing ZmMs45pro:OsMs45 (FIG. 1E).

### DETAILED DESCRIPTION

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

Mutations that cause male sterility in plants have the potential to be useful in methods for hybrid seed production. For example, use of a male-sterile female inbred plant as a parent to produce hybrid seed can lower production costs by eliminating the need for the labor-intensive removal of male flowers and self-pollination of the female inbred. Emasculation of wheat can be especially challenging since the male flowers and female flowers are located within the same floret. This makes it difficult to prevent self-pollination of the female and fertilize it with pollen from another wheat plant. Self-pollination results in seed of the female inbred being harvested along with the hybrid seed which is normally produced. Female inbred seed does not exhibit heterosis and therefore is not as commercially desirable as F1 seed. Thus, use of a male-sterile female inbred prevents self-fertilization while maintaining the purity of hybrid seeds.

Mutations that cause male sterility in crop plants such as maize, wheat and rice have been produced by a variety of methods such as X-rays or UV-irradiations, chemical treatments, or transposable element insertions (ms23, ms25, ms26, ms32) (Chaubal et al. 2000) Am J Bot 87:1193-1201). However, such methods are random mutagenesis methods that induce mutations randomly throughout the genome and not just in the gene of interest. Typically, with such random mutagenesis methods, it requires considerable effort to identify a plant that contains a mutation in the gene of interest and it is by no means certain that such a plant will be identified. Furthermore, with random mutagenesis methods, each plant tested is likely to carry multiple mutations. Therefore, a plant that is identified with the mutation in the gene of interest must be backcrossed for several or more generations to eliminate the undesired mutations.

In contrast to such random mutagenesis methods, the described herein are methods for producing male sterile plants by introducing a genetic modification into at least one or more endogenous fertility genes, such as MS9, MS22, MS26 or MS45 polynucleotide sequences, in a plant cell. The introduced genetic modification confers male sterility to a plant arising from the plant cell. Preferably the plant is a crop plant.

U.S. Patent publication US 20150191743 A1, published Jul. 9, 2015, describes a male fertility gene referred to as “MS9” that is located on maize chromosome 1 and encodes a myb transcription factor critical to male fertility. The Ms9 phenotype was first identified in maize in 1932. Beadle, (1932) Genetics 17:413-431. It was found to be linked to the P1 gene on Chromosome 1. Breakdown of male reproductive tissue development occurs very early in premeiosis; tapetal cells may be affected as well. Greyson, et al., (1980) Can. J. Genet. Cytol. 22:153-166. Examples of genomic DNA and polypeptide sequences of maize Ms9 are disclosed in US patent publication 20150191743, published on Jul. 9, 2015.

U.S. Patent publication US 2009-0038026 A1, published Feb. 5, 2009, describes a male fertile gene referred to as “Msca1” or “MS22” that is located on maize chromosome 7 and encodes a protein critical to male fertility. Mutations referred to as ms22 or msca1 were first noted as phenotypically male sterile with anthers which did not extrude from the tassel and lacked sporogenous tissue. West and Albertsen (1985) Maize Newsletter 59:87; Neuffer et al. (1977) Mutants of maize. Cold Spring Harbor Laboratory Press, Cold Spring Harbor, NY. The mutant locus was originally referred to as ms22 but was later changed to msca1, or male sterile converted anther. See Chaubal et al. “The transformation of anthers in the msca1 mutant of maize” Planta (2003)216:778-788. Examples of genomic DNA and polypeptide sequences of wheat Ms22 are disclosed in US patent publication 2014/007559, see for example U.S. Pat. No. 7,919,676).

U.S. Pat. No. 7,517,975, issued Apr. 14, 2009, describes a male fertile gene referred to as “MS26” (also known as SB200 or SBMu200) that is located on maize chromosome 1. MS26 can be used in the systems described above, and other systems impacting male fertility. The term “wheat Ms26 gene” or similar reference means a gene or sequence in wheat that is orthologous to Ms26 in maize or rice, e.g. as disclosed in U.S. Pat. No. 7,919,676 or 8,293,970. Examples of genomic DNA and polypeptide sequences of wheat Ms26 are disclosed in US patent publication 2014/0075597, U.S. Pat. Nos. 7,098,388, 7,517,975, 7,612,251 and described elsewhere herein.

U.S. Pat. No. 5,478,369 issued Dec. 26, 1995 describes a male fertile gene referred to as “MS45” cloned on maize chromosome 9. Examples of genomic DNA and polypeptide sequences of wheat Ms45 are disclosed in US patent publication 2014/0075597, see for example U.S. Pat. No. 6,265,640.

Additionally, the present disclosure includes the following MS9, MS22, MS26, and MS45 polynucleotides and polypeptides:

TABLE 1
Summary of SEQ ID NOs:

| SEQ ID NO: | Description |
|---|---|
| 1 | Wheat Ms9 4AS genomic |
| | (exon/intron) |
| 2 | Wheat Ms9 4AS promoter |
| 3 | Wheat Ms9 4AS coding |
| 4 | Wheat Ms9 4AS amino acid |
| 5 | Wheat Ms9 4BL genomic |
| | (exon/intron) |
| 6 | Wheat Ms9 4BL promoter |
| 7 | Wheat Ms9 4BL coding |
| 8 | Wheat Ms9 4BL amino acid |
| 9 | Wheat Ms9 4DL genomic |
| | (exon/intron) |
| 10 | Wheat Ms9 4DL promoter |
| 11 | Wheat Ms9 4DL coding |
| 12 | Wheat Ms9 4DL amino acid |
| 13 | Maize Ms9 genomic (exon/intron) |
| 14 | Maize Ms9 promoter |
| 15 | Maize Ms9 coding |
| 16 | Maize Ms9 amino acid |
| 17 | Rice Ms9 genomic (exon/intron) |
| 18 | Rice Ms9 promoter |
| 19 | Rice Ms9 coding |
| 20 | Rice Ms9 amino acid |
| 21 | Wheat Ms22 AL genomic |
| | (exon/intron) |
| 22 | Wheat Ms22 AL promoter |
| 23 | Wheat Ms22 AL coding |
| 24 | Wheat Ms22 AL amino acid |
| 25 | Wheat Ms22 BL genomic |
| | (exon/intron) |
| 26 | Wheat Ms22 BL promoter |
| 27 | Wheat Ms22 BL coding |
| 28 | Wheat Ms22 BL amino acid |
| 29 | Wheat Ms22 DL genomic |
| | (exon/intron) |
| 30 | Wheat Ms22 DL promoter |
| 31 | Wheat Ms22 DL coding |
| 32 | Wheat Ms22 DL amino acid |
| 33 | Maize Ms22 genomic (exon/intron) |
| 34 | Maize Ms22 promoter |
| 35 | Maize Ms22 coding |
| 36 | Maize Ms22 amino acid |
| 37 | Rice Ms22 genomic (exon/intron) |
| 38 | Rice Ms22 promoter |
| 39 | Rice Ms22 coding |
| 40 | Rice Ms22 amino acid |
| 41 | Wheat Ms26 4A5 genomic |
| | (exon/intron) |
| 42 | Wheat Ms26 4A5 promoter |
| 43 | Wheat Ms26 4A5 coding |
| 44 | Wheat Ms26 4A5 amino acid |
| 45 | Wheat Ms26 4BL genomic |
| | (exon/intron) |
| 46 | Wheat Ms26 4BL promoter |
| 47 | Wheat Ms26 4BL coding |
| 48 | Wheat Ms26 4BL amino acid |
| 49 | Wheat Ms26 4DL genomic |
| | (exon/intron) |
| 50 | Wheat Ms26 4DL promoter |
| 51 | Wheat Ms26 4DL coding |
| 52 | Wheat Ms26 4DL amino acid |
| 53 | Maize Ms26 genomic (exon/intron) |
| 54 | Maize Ms26 promoter |
| 55 | Maize Ms26 coding |
| 56 | Maize Ms26 amino acid |
| 57 | Rice Ms26 genomic (exon/intron) |
| 58 | Rice Ms26 promoter |
| 59 | Rice Ms26 coding |
| 60 | Rice Ms26 amino acid |
| 61 | Wheat Ms45 4AS genomic |
| | (exon/intron) |
| 62 | Wheat Ms45 4AS promoter |
| 63 | Wheat Ms45 4AS coding |
| 64 | Wheat Ms45 4A5 amino acid |
| 65 | Wheat Ms45 4BL genomic |
| | (exon/intron) |
| 66 | Wheat Ms45 4BL promoter |
| 67 | Wheat Ms45 4BL coding |
| 68 | Wheat Ms45 4BL amino acid |
| 69 | Wheat Ms45 4DL genomic |
| | (exon/intron) |
| 70 | Wheat Ms45 4DL promoter |
| 71 | Wheat Ms45 4DL coding |
| 72 | Wheat Ms45 4DL amino acid |
| 73 | Maize Ms45 genomic (exon/intron) |
| 74 | Maize Ms45 promoter |
| 75 | Maize Ms45 coding |
| 76 | Maize Ms45 amino acid |
| 77 | Rice Ms45 genomic (exon/intron) |
| 78 | Rice Ms45 promoter |
| 79 | Rice Ms45 coding |
| 80 | Rice Ms45 amino acid |
| 81 | Wheat Ms9 4BL CR1 target |
| 82 | Wheat Ms9 4BL CR2 target |
| 83 | Wheat Ms9 4BL CR3 target |
| 84 | Wheat Ms9 4BL CR4 target |
| 85 | Wheat Ms9 4BL CR5 target |
| 86 | Wheat Ms9 4BL CR6 target |
| 87 | Wheat Ms9 4BL CR7 target |
| 88 | Wheat Ms9 4BL CR8 target |
| 89 | Wheat Ms9 4BL CR9 target |
| 90 | Wheat Ms9 4BL CR10 target |
| 91 | Wheat Ms9 4BL CR11 target |
| 92 | Wheat Ms9 4BL CR12 target |
| 93 | Wheat Ms9 4BL CR13 target |
| 94 | Wheat Ms9 4BL CR14 target |
| 95 | Wheat Ms9 4BL CR15 target |
| 96 | Maize Ms9 CR1 target |
| 97 | Maize Ms9 CR2 target |
| 98 | Maize Ms9 CR3 target |
| 99 | Maize Ms9 CR4 target |
| 100 | Maize Ms9 CR5 target |
| 101 | Maize Ms9 CR6 target |
| 102 | Maize Ms9 CR7 target |
| 103 | Maize Ms9 CR8 target |
| 104 | Maize Ms9 CR9 target |
| 105 | Maize Ms9 CR10 target |
| 106 | Maize Ms9 CR11 target |
| 107 | Maize Ms9 CR12 target |
| 108 | Maize Ms9 CR13 target |
| 109 | Maize Ms9 CR14 target |
| 110 | Maize Ms9 CR15 target |
| 111 | Rice Ms9 CR1 target |
| 112 | Rice Ms9 CR2 target |
| 113 | Rice Ms9 CR3 target |
| 114 | Rice Ms9 CR4 target |
| 115 | Rice Ms9 CR5 target |
| 116 | Rice Ms9 CR6 target |
| 117 | Rice Ms9 CR7 target |
| 118 | Rice Ms9 CR8 target |
| 119 | Rice Ms9 CR9 target |
| 120 | Rice Ms9 CR10 target |
| 121 | Rice Ms9 CR11 target |
| 122 | Rice Ms9 CR12 target |
| 123 | Rice Ms9 CR13 target |
| 124 | Rice Ms9 CR14 target |
| 125 | Rice Ms9 CR15 target |
| 126 | Wheat Ms22CR1 target |
| 127 | Wheat Ms22CR2 target |
| 128 | Wheat Ms22CR3 target |
| 129 | Wheat Ms22CR4 target |
| 130 | Wheat Ms22CR5 target |
| 131 | Wheat Ms22CR6 target |
| 132 | Wheat Ms22CR7 target |
| 133 | Wheat Ms22CR8 target |
| 134 | Wheat Ms22CR9 target |
| 135 | Wheat Ms22CR10 target |
| 136 | Wheat Ms22CR11 target |
| 137 | Wheat Ms22CR12 target |
| 138 | Wheat Ms22CR13 target |
| 139 | Wheat Ms22CR14 target |
| 140 | Wheat Ms22CR15 target |
| 141 | Maize Ms22 CR1 target |
| 142 | Maize Ms22 CR2 target |
| 143 | Maize Ms22 CR3 target |
| 144 | Maize Ms22 CR4 target |
| 145 | Maize Ms22 CR5 target |
| 146 | Maize Ms22 CR6 target |
| 147 | Maize Ms22 CR7 target |
| 148 | Maize Ms22 CR8 target |
| 149 | Maize Ms22 CR9 target |
| 150 | Maize Ms22 CR10 target |
| 151 | Maize Ms22 CR11 target |
| 152 | Maize Ms22 CR12 target |
| 153 | Maize Ms22 CR13 target |
| 154 | Maize Ms22 CR14 target |
| 155 | Maize Ms22 CR15 target |
| 156 | Rice Ms22 CR1 target |
| 157 | Rice Ms22 CR2 target |
| 158 | Rice Ms22 CR3 target |
| 159 | Rice Ms22 CR4 target |
| 160 | Rice Ms22 CR5 target |
| 161 | Rice Ms22 CR6 target |
| 162 | Rice Ms22 CR7 target |
| 163 | Rice Ms22 CR8 target |
| 164 | Rice Ms22 CR9 target |
| 165 | Rice Ms22 CR10 target |
| 166 | Rice Ms22 CR11 target |
| 167 | Rice Ms22 CR12 target |
| 168 | Rice Ms22 CR13 target |
| 169 | Rice Ms22 CR14 target |
| 170 | Rice Ms22 CR15 target |
| 171 | Wheat Ms26 CR1 target |
| 172 | Wheat Ms26 CR2 target |
| 173 | Wheat Ms26 CR3 target |
| 174 | Wheat Ms26 CR4 target |
| 175 | Wheat Ms26 CR5 target |
| 176 | Wheat Ms26 CR6 target |
| 177 | Wheat Ms26 CR7 target |
| 178 | Wheat Ms26 CR8 target |
| 179 | Wheat Ms26 CR9 target |
| 180 | Wheat Ms26 CR10 target |
| 181 | Wheat Ms26 CR11 target |
| 182 | Wheat Ms26 CR12 target |
| 183 | Wheat Ms26 CR13 target |
| 184 | Wheat Ms26 CR14 target |
| 185 | Wheat Ms26 CR15 target |
| 186 | Maize Ms26 CR1 target |
| 187 | Maize Ms26 CR2 target |
| 188 | Maize Ms26 CR3 target |
| 189 | Maize Ms26 CR4 target |
| 190 | Maize Ms26 CR5 target |
| 191 | Maize Ms26 CR6 target |
| 192 | Maize Ms26 CR7 target |
| 193 | Maize Ms26 CR8 target |
| 194 | Maize Ms26 CR9 target |
| 195 | Maize Ms26 CR10 target |
| 196 | Maize Ms26 CR11 target |
| 197 | Maize Ms26 CR12 target |
| 198 | Maize Ms26 CR13 target |
| 199 | Maize Ms26 CR14 target |
| 200 | Maize Ms26 CR15 target |
| 201 | Maize Ms26 CR16 target |
| 202 | Maize Ms26 CR17 target |
| 203 | Maize Ms26 CR18 target |
| 204 | Rice Ms26 CR1 target |
| 205 | Rice Ms26 CR2 target |
| 206 | Rice Ms26 CR3 target |
| 207 | Rice Ms26 CR4 target |
| 208 | Rice Ms26 CR5 target |
| 209 | Rice Ms26 CR6 target |
| 210 | Rice Ms26 CR7 target |
| 211 | Rice Ms26 CR8 target |
| 212 | Rice Ms26 CR9 target |
| 213 | Rice Ms26 CR10 target |
| 214 | Rice Ms26 CR11 target |
| 215 | Rice Ms26 CR12 target |
| 216 | Wheat Ms45 CR1 target |
| 217 | Wheat Ms45 CR2 target |
| 218 | Wheat Ms45 CR3 target |
| 219 | Wheat Ms45 CR4 target |
| 220 | Wheat Ms45 CR5 target |
| 221 | Wheat Ms45 CR6 target |
| 222 | Wheat Ms45 CR7 target |
| 223 | Wheat Ms45 CR8 target |
| 224 | Wheat Ms45 CR9 target |
| 225 | Wheat Ms45 CR10 target |
| 226 | Wheat Ms45 CR11 target |
| 227 | Wheat Ms45 CR12 target |
| 228 | Wheat Ms45 CR13 target |
| 229 | Wheat Ms45 CR14 target |
| 230 | Wheat Ms45 CR15 target |
| 231 | Wheat Ms45 CR16 target |
| 232 | Maize Ms45 CR1 target |
| 233 | Maize Ms45 CR2 target |
| 234 | Maize Ms45 CR3 target |
| 235 | Maize Ms45 CR4 target |
| 236 | Maize Ms45 CR5 target |
| 237 | Maize Ms45 CR6 target |
| 238 | Maize Ms45 CR7 target |
| 239 | Maize Ms45 CR8 target |
| 240 | Maize Ms45 CR9 target |
| 241 | Maize Ms45 CR10 target |
| 242 | Maize Ms45 CR11 target |
| 243 | Maize Ms45 CR12 target |
| 244 | Maize Ms45 CR13 target |
| 245 | Maize Ms45 CR14 target |
| 246 | Maize Ms45 CR15 target |
| 247 | Maize Ms45 CR16 target |
| 248 | Maize Ms45 CR17 target |
| 249 | Maize Ms45 CR18 target |
| 250 | Rice Ms45 CR1 target |
| 251 | Rice Ms45 CR2 target |
| 252 | Rice Ms45 CR3 target |
| 253 | Rice Ms45 CR4 target |
| 254 | Rice Ms45 CR5 target |
| 255 | Rice Ms45 CR6 target |
| 256 | Rice Ms45 CR7 target |
| 257 | Rice Ms45 CR8 target |
| 258 | Rice Ms45 CR9 target |
| 259 | Rice Ms45 CR10 target |
| 260 | Rice Ms45 CR11 target |
| 261 | Rice Ms45 CR12 target |
| 262 | Rice Ms45 CR13 target |
| 263 | Rice Ms45 CR14 target |
| 264 | Rice Ms45 CR15 target |
| 265 | Wheat MS45 wildtype |
| 266 | Wheat MS45 A genome mutation |
| 267 | Wheat Ms9 4BL CR1 guide |
| 268 | Wheat Ms9 4BL CR2 guide |
| 269 | Wheat Ms9 4BL CR3 guide |
| 270 | Wheat Ms9 4BL CR4 guide |
| 271 | Wheat Ms9 4BL CR5 guide |
| 272 | Wheat Ms9 4BL CR6 guide |
| 273 | Wheat Ms9 4BL CR7 guide |
| 274 | Wheat Ms9 4BL CR8 guide |
| 275 | Wheat Ms9 4BL CR9 guide |
| 276 | Wheat Ms9 4BL CR10 guide |
| 277 | Wheat Ms9 4BL CR11 guide |
| 278 | Wheat Ms9 4BL CR12 guide |
| 279 | Wheat Ms9 4BL CR13 guide |
| 280 | Wheat Ms9 4BL CR14 guide |
| 281 | Wheat Ms9 4BL CR15 guide |
| 282 | Maize Ms9 CR1 guide |
| 283 | Maize Ms9 CR2 guide |
| 284 | Maize Ms9 CR3 guide |
| 285 | Maize Ms9 CR4 guide |
| 286 | Maize Ms9 CR5 guide |
| 287 | Maize Ms9 CR6 guide |
| 288 | Maize Ms9 CR7 guide |
| 289 | Maize Ms9 CR8 guide |
| 290 | Maize Ms9 CR9 guide |
| 291 | Maize Ms9 CR10 guide |
| 292 | Maize Ms9 CR11 guide |
| 293 | Maize Ms9 CR12 guide |
| 294 | Maize Ms9 CR13 guide |
| 295 | Maize Ms9 CR14 guide |
| 296 | Maize Ms9 CR15 guide |
| 297 | Rice Ms9 CR1 guide |
| 298 | Rice Ms9 CR2 guide |
| 299 | Rice Ms9 CR3 guide |
| 300 | Rice Ms9 CR4 guide |
| 301 | Rice Ms9 CR5 guide |
| 302 | Rice Ms9 CR6 guide |
| 303 | Rice Ms9 CR7 guide |
| 304 | Rice Ms9 CR8 guide |
| 305 | Rice Ms9 CR9 guide |
| 306 | Rice Ms9 CR10 guide |
| 307 | Rice Ms9 CR11 guide |
| 308 | Rice Ms9 CR12 guide |
| 309 | Rice Ms9 CR13 guide |
| 310 | Rice Ms9 CR14 guide |
| 311 | Rice Ms9 CR15 guide |
| 312 | Wheat Ms22CR1 guide |
| 313 | Wheat Ms22CR2 guide |
| 314 | Wheat Ms22CR3 guide |
| 315 | Wheat Ms22CR4 guide |
| 316 | Wheat Ms22CR5 guide |
| 317 | Wheat Ms22CR6 guide |
| 318 | Wheat Ms22CR7 guide |
| 319 | Wheat Ms22CR8 guide |
| 320 | Wheat Ms22CR9 guide |
| 321 | Wheat Ms22CR10 guide |
| 322 | Wheat Ms22CR11 guide |
| 323 | Wheat Ms22CR12 guide |
| 324 | Wheat Ms22CR13 guide |
| 325 | Wheat Ms22CR14 guide |
| 326 | Wheat Ms22CR15 guide |
| 327 | Maize Ms22 CR1 guide |
| 328 | Maize Ms22 CR2 guide |
| 329 | Maize Ms22 CR3 guide |
| 330 | Maize Ms22 CR4 guide |
| 331 | Maize Ms22 CR5 guide |
| 332 | Maize Ms22 CR6 guide |
| 333 | Maize Ms22 CR7 guide |
| 334 | Maize Ms22 CR8 guide |
| 335 | Maize Ms22 CR9 guide |
| 336 | Maize Ms22 CR10 guide |
| 337 | Maize Ms22 CR11 guide |
| 338 | Maize Ms22 CR12 guide |
| 339 | Maize Ms22 CR13 guide |
| 340 | Maize Ms22 CR14 guide |
| 341 | Maize Ms22 CR15 guide |
| 342 | Rice Ms22 CR1 guide |
| 343 | Rice Ms22 CR2 guide |
| 344 | Rice Ms22 CR3 guide |
| 345 | Rice Ms22 CR4 guide |
| 346 | Rice Ms22 CR5 guide |
| 347 | Rice Ms22 CR6 guide |
| 348 | Rice Ms22 CR7 guide |
| 349 | Rice Ms22 CR8 guide |
| 350 | Rice Ms22 CR9 guide |
| 351 | Rice Ms22 CR10 guide |
| 352 | Rice Ms22 CR11 guide |
| 353 | Rice Ms22 CR12 guide |
| 354 | Rice Ms22 CR13 guide |
| 355 | Rice Ms22 CR14 guide |
| 356 | Rice Ms22 CR15 guide |
| 357 | Wheat Ms26 CR1 guide |
| 358 | Wheat Ms26 CR2 guide |
| 359 | Wheat Ms26 CR3 guide |
| 360 | Wheat Ms26 CR4 guide |
| 361 | Wheat Ms26 CR5 guide |
| 362 | Wheat Ms26 CR6 guide |
| 363 | Wheat Ms26 CR7 guide |
| 364 | Wheat Ms26 CR8 guide |
| 365 | Wheat Ms26 CR9 guide |
| 366 | Wheat Ms26 CR10 guide |
| 367 | Wheat Ms26 CR11 guide |
| 368 | Wheat Ms26 CR12 guide |
| 369 | Wheat Ms26 CR13 guide |
| 370 | Wheat Ms26 CR14 guide |
| 371 | Wheat Ms26 CR15 guide |
| 372 | Maize Ms26 CR1 guide |
| 373 | Maize Ms26 CR2 guide |
| 374 | Maize Ms26 CR3 guide |
| 375 | Maize Ms26 CR4 guide |
| 376 | Maize Ms26 CR5 guide |
| 377 | Maize Ms26 CR6 guide |
| 378 | Maize Ms26 CR7 guide |
| 379 | Maize Ms26 CR8 guide |
| 380 | Maize Ms26 CR9 guide |
| 381 | Maize Ms26 CR10 guide |
| 382 | Maize Ms26 CR11 guide |
| 383 | Maize Ms26 CR12 guide |
| 384 | Maize Ms26 CR13 guide |
| 385 | Maize Ms26 CR14 guide |
| 386 | Maize Ms26 CR15 guide |
| 387 | Maize Ms26 CR16 guide |
| 388 | Maize Ms26 CR17 guide |
| 389 | Maize Ms26 CR18 guide |
| 390 | Rice Ms26 CR1 guide |
| 391 | Rice Ms26 CR2 guide |
| 392 | Rice Ms26 CR3 guide |
| 393 | Rice Ms26 CR4 guide |
| 394 | Rice Ms26 CR5 guide |
| 395 | Rice Ms26 CR6 guide |
| 396 | Rice Ms26 CR7 guide |
| 397 | Rice Ms26 CR8 guide |
| 398 | Rice Ms26 CR9 guide |
| 399 | Rice Ms26 CR10 guide |
| 400 | Rice Ms26 CR11 guide |
| 401 | Rice Ms26 CR12 guide |
| 402 | Wheat Ms45 CR1 guide |
| 403 | Wheat Ms45 CR2 guide |
| 404 | Wheat Ms45 CR3 guide |
| 405 | Wheat Ms45 CR4 guide |
| 406 | Wheat Ms45 CR5 guide |
| 407 | Wheat Ms45 CR6 guide |
| 408 | Wheat Ms45 CR7 guide |
| 409 | Wheat Ms45 CR8 guide |
| 410 | Wheat Ms45 CR9 guide |
| 411 | Wheat Ms45 CR10 guide |
| 412 | Wheat Ms45 CR11 guide |
| 413 | Wheat Ms45 CR12 guide |
| 414 | Wheat Ms45 CR13 guide |
| 415 | Wheat Ms45 CR14 guide |
| 416 | Wheat Ms45 CR15 guide |
| 417 | Wheat Ms45 CR16 guide |
| 418 | Maize Ms45 CR1 guide |
| 419 | Maize Ms45 CR2 guide |
| 420 | Maize Ms45 CR3 guide |
| 421 | Maize Ms45 CR4 guide |
| 422 | Maize Ms45 CR5 guide |
| 423 | Maize Ms45 CR6 guide |
| 424 | Maize Ms45 CR7 guide |
| 425 | Maize Ms45 CR8 guide |
| 426 | Maize Ms45 CR9 guide |
| 427 | Maize Ms45 CR10 guide |
| 428 | Maize Ms45 CR11 guide |
| 429 | Maize Ms45 CR12 guide |
| 430 | Maize Ms45 CR13 guide |
| 431 | Maize Ms45 CR14 guide |
| 432 | Maize Ms45 CR15 guide |
| 433 | Maize Ms45 CR16 guide |
| 434 | Maize Ms45 CR17 guide |
| 435 | Maize Ms45 CR18 guide |
| 436 | Rice Ms45 CR1 guide |
| 437 | Rice Ms45 CR2 guide |
| 438 | Rice Ms45 CR3 guide |
| 439 | Rice Ms45 CR4 guide |
| 440 | Rice Ms45 CR5 guide |
| 441 | Rice Ms45 CR6 guide |
| 442 | Rice Ms45 CR7 guide |
| 443 | Rice Ms45 CR8 guide |
| 444 | Rice Ms45 CR9 guide |
| 445 | Rice Ms45 CR10 guide |
| 446 | Rice Ms45 CR11 guide |
| 447 | Rice Ms45 CR12 guide |
| 448 | Rice Ms45 CR13 guide |
| 449 | Rice Ms45 CR14 guide |
| 450 | Rice Ms45 CR15 guide |
| 451 | Wheat U6 RNA polymerase III promoter |
| 452 | Wheat U6 RNA polymerase III promoter |
| 453 | Wheat U6 RNA polymerase III promoter |

An isolated Ms9 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 4, 8, 12, 16, or 20; or (ii) a full complement of the nucleic acid sequence of (i), wherein the full complement and the nucleic acid sequence of (i) consist of the same number of nucleotides and are 100% complementary.

An isolated Ms9 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 4, 8, 12, 16, or 20.

An isolated Ms9 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 1-3, 5-7, 9-11, 13-15, or 17-19 and combinations thereof; or (ii) a full complement of the nucleic acid sequence of (i).

An isolated Ms9 polynucleotide comprising a nucleotide sequence, wherein the nucleotide sequence is hybridizable under stringent conditions with a DNA molecule comprising the full complement of SEQ ID NO: 1-3, 5-7, 9-11, 13-15, or 17-19. The isolated MS9 protein of the present disclosure may also be a protein which is encoded by a nucleic acid comprising a nucleotide sequence hybridizable under stringent conditions with the complementary strand of the nucleotide sequence of SEQ ID NO: 1, 3, 5, 7, 9, 11, 13, 15, 17, or 19.

An isolated Ms9 polynucleotide comprising a nucleotide sequence, wherein the nucleotide sequence is derived from SEQ ID NO: 1-3, 5-7, 9-11, 13-15, or 17-19 by alteration of one or more nucleotides by at least one method selected from the group consisting of: deletion, substitution, addition and insertion.

An isolated Ms9 polynucleotide comprising a nucleotide sequence, wherein the nucleotide sequence corresponds to an allele of SEQ ID NO: 1, 5, 9, 13, or 17.

An isolated Ms22 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 24, 28, 32, 36, or 40; or (ii) a full complement of the nucleic acid sequence of (i), wherein the full complement and the nucleic acid sequence of (i) consist of the same number of nucleotides and are 100% complementary.

An isolated Ms22 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 24, 28, 32, 36, or 40.

An isolated Ms22 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 21-23, 25-27, 29-31, 33-35, or 37-39 and combinations thereof; or (ii) a full complement of the nucleic acid sequence of (i).

An isolated Ms22 polynucleotide comprising a nucleotide sequence, wherein the nucleotide sequence is hybridizable under stringent conditions with a DNA molecule comprising the full complement of SEQ ID NO: 21-23, 25-27, 29-31, 33-35, or 37-39. The isolated MS22 protein of the present disclosure may also be a protein which is encoded by a nucleic acid comprising a nucleotide sequence hybridizable under stringent conditions with the complementary strand of the nucleotide sequence of SEQ ID NO: 21, 23, 25, 27, 29, 31, 33, 35, 37, or 39.

An isolated Ms22 polynucleotide comprising a nucleotide sequence, wherein the nucleotide sequence is derived from SEQ ID NO: 21-23, 25-27, 29-31, 33-35, or 37-39 by alteration of one or more nucleotides by at least one method selected from the group consisting of: deletion, substitution, addition and insertion.

An isolated Ms22 polynucleotide comprising a nucleotide sequence, wherein the nucleotide sequence corresponds to an allele of SEQ ID NO: 21, 25, 29, 33, or 37.

An isolated MS26 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 44, 48, 52, 56, or 60; or (ii) a full complement of the nucleic acid sequence of (i), wherein the full complement and the nucleic acid sequence of (i) consist of the same number of nucleotides and are 100% complementary.

An isolated MS26 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 44, 48, 52, 56, or 60.

An isolated MS26 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 41-43, 45-47, 49-51, 53-55, or 57-59 and combinations thereof; or (ii) a full complement of the nucleic acid sequence of (i).

An isolated MS26 polynucleotide comprising a nucleotide sequence, wherein the nucleotide sequence is hybridizable under stringent conditions with a DNA molecule comprising the full complement of SEQ ID NO: 41-43, 45-47, 49-51, 53-55, or 57-59. The isolated MS26 protein of the present disclosure may also be a protein which is encoded by a nucleic acid comprising a nucleotide sequence hybridizable under stringent conditions with the complementary strand of the nucleotide sequence of SEQ ID NO: 41, 43 45, 47, 49, 51, 53, 55, 57, or 59.

An isolated MS26 polynucleotide comprising a nucleotide sequence, wherein the nucleotide sequence is derived from SEQ ID NO: 41-43, 45-47, 49-51, 53-55, or 57-59 by alteration of one or more nucleotides by at least one method selected from the group consisting of: deletion, substitution, addition and insertion.

An isolated MS26 polynucleotide comprising a nucleotide sequence, wherein the nucleotide sequence corresponds to an allele of SEQ ID NO: 41, 45, 49, 53, or 57.

An isolated MS45 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 64, 68, 72, 76 or 80; or (ii) a full complement of the nucleic acid sequence of (i), wherein the full complement and the nucleic acid sequence of (i) consist of the same number of nucleotides and are 100% complementary.

An isolated MS45 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 64, 68, 72, 76 or 80.

An isolated MS45 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 61-63, 65-67, 69-71, 73-75, or 77-79 and combinations thereof; or (ii) a full complement of the nucleic acid sequence of (i).

An isolated MS45 polynucleotide comprising a nucleotide sequence, wherein the nucleotide sequence is hybridizable under stringent conditions with a DNA molecule comprising the full complement of SEQ ID NO: 61-63, 65-67, 69-71, 73-75, or 77-79. The isolated MS45 protein of the present disclosure may also be a protein which is encoded by a nucleic acid comprising a nucleotide sequence hybridizable under stringent conditions with the complementary strand of the nucleotide sequence of SEQ ID NO: 61, 63, 65, 67, 69, 71, 73, 75, 77, or 79.

An isolated MS45 polynucleotide comprising a nucleotide sequence, wherein the nucleotide sequence is derived from SEQ ID NO: 61-63, 65-67, 69-71, 73-75, or 77-79 by alteration of one or more nucleotides by at least one method selected from the group consisting of: deletion, substitution, addition and insertion.

An isolated MS45 polynucleotide comprising a nucleotide sequence, wherein the nucleotide sequence corresponds to an allele of SEQ ID NO: 61, 65, 69, 73, or 77.

Any of the Ms9, Ms22, Ms26, or Ms45 polynucleotides and polypeptide described herein and known in the art may be utilized in any methods and compositions of the present disclosure.

Because the genetic modification is introduced at a target site located in or near a male fertility gene of MS9, MS22, MS26, or MS45, it is not necessary to screen a population of thousands of plants carrying random mutations, such as those resulting from chemical mutagenesis, in order to identify a plant with the introduced genetic modification. Therefore, the need to backcross a plant to remove undesired mutations that are not the introduced genetic modification is eliminated or at least reduced.

Described herein are compositions and methods for producing male-sterile plants that introduce a genetic modification into a male fertility gene locus of MS9, MS22, MS26 or MS45 in the plant genome in a plant cell and obtaining a plant from that plant cell. The methods may employ a guide RNA/Cas endonuclease system, wherein the Cas endonuclease is guided by the guide RNA to recognize and optionally introduce a double strand break at a specific target site into the genome of a cell. The guide RNA/Cas endonuclease system provides for an effective system for modifying target sites within the genome of a plant, plant cell or seed. The target site recognized by a Cas endonuclease may be located within or outside the MS9, MS22, MS26 or MS45 polynucleotide sequence, for example, within or outside the MS9, MS22, MS26 or MS45 gene locus.

In one embodiment, the method comprises a method for producing a male-sterile plant, the method comprising: a) obtaining a first plant comprising at least one Cas endonuclease capable of introducing a double strand break at a genomic target site located in a male fertility gene locus of MS9, MS22, MS26 or MS45 in the plant genome; b) obtaining a second plant comprising a guide RNA that is capable of forming a complex with the Cas endonuclease of (a),c) crossing the first plant of (a) with the second plant of (b); d) evaluating the progeny of (c) for an alteration in the target site; and e) selecting a progeny plant that is male-sterile.

Compositions and methods are also provided for editing a nucleotide sequence in the genome of a cell. In one embodiment, the disclosure describes a method for editing a nucleotide sequence located in or near a male fertility gene of MS9, MS22, MS26, or MS45 in the genome of a plant cell, the method comprising providing a guide RNA, a polynucleotide modification template, and at least one maize optimized Cas9 endonuclease to a plant cell, wherein the maize optimized Cas9 endonuclease is capable of introducing a double-strand break at a target site in the plant genome, wherein said polynucleotide modification template includes at least one nucleotide modification of said nucleotide sequence. The nucleotide to be edited (the nucleotide sequence of interest) can be located within or outside a target site located in or near a male fertility gene of MS9, MS22, MS26, or MS45 that is recognized and cleaved by a Cas endonuclease. Cells include, but are not limited to, plant cells as well as plants and seeds produced by the methods described herein.

Compositions and methods are also provided for methods of modifying the male-fertility of a plant, the method comprising introducing at least one guide RNA, at least one polynucleotide modification template and at least one Cas endonuclease into a cell. The Cas endonuclease introduces a double-strand break at a target site located in or near a MS9, MS22, MS26, or MS45 gene in the genome of said plant cell and the polynucleotide modification template comprises at least one nucleotide modification of a nucleotide sequence at the target site located in or near a male fertility gene of MS9, MS22, MS26, or MS45 that decreases the expression level of the MS9, MS22, MS26 or MS45 gene, to produce a male-sterile plant.

In another embodiment, the methods include selecting a male-sterile plant, the method comprising selecting at least one male-sterile plant that comprises the introduced genetic modification(s) in at least one or more of the endogenous MS9, MS22, MS26 or MS45 polynucleotide sequences or MS9, MS22, MS26 or MS45 gene locus. Also provided is a plant cell or plant or seed obtained or produced from the methods described herein.

The plant in the embodiments described herein is a monocot or a dicot. More specifically, the monocot is selected from the group consisting of maize, rice, sorghum, rye, barley, wheat, millet, oats, sugarcane, turfgrass, or switchgrass. The dicot is selected from the group consisting of soybean, canola, alfalfa, sunflower, cotton, tobacco, peanut, potato, tobacco, Arabidopsis, or safflower.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

A Cas gene includes a gene that is generally coupled, associated or close to or in the vicinity of flanking CRISPR loci. The terms “Cas gene”, “CRISPR-associated (Cas) gene” are used interchangeably herein. A comprehensive review of the Cas protein family is presented in Haft et al. (2005) Computational Biology, PLOS Comput Biol 1(6): e60. doi:10.1371/journal.pcbi.0010060.

Cas endonuclease relates to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell. As used herein, the term “guide polynucleotide/Cas endonuclease system” includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

In one embodiment, the Cas endonuclease gene is a Cas9 endonuclease, such as but not limited to, Cas9 genes listed in SEQ ID NOs: 462, 474, 489, 494, 499, 505, and 518 of WO2007/025097 published Mar. 1, 2007, and incorporated herein by reference. In another embodiment, the Cas endonuclease gene is plant optimized Cas9 endonuclease, for example, codon-optimized for expression in maize, wheat, or soybean. In another embodiment, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. As used herein, “operably linked” is intended to mean a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked means that the coding regions are in the same reading frame. In one embodiment, the Cas endonuclease gene is a Cas9 endonuclease gene of SEQ ID NO:1, 124, 212, 213, 214, 215, 216, 193 or nucleotides 2037-6329 of SEQ ID NO:5, or any functional fragment or variant thereof of US publication number 20160208272, published Jul. 26, 2016, and incorporated herein by reference.

The terms “functional fragment”, “fragment that is functionally equivalent” and “functionally equivalent fragment” are used interchangeably herein. These terms refer to a portion or subsequence of the polypeptide sequence of the present disclosure in which the polypeptide's native function is retained.

The terms “functional variant”, “Variant that is functionally equivalent” and “functionally equivalent variant” are used interchangeably herein. These terms refer to a variant of a polypeptide of the present disclosure in which which the polypeptide's native function is retained. Fragments and variants can be obtained via methods such as site-directed mutagenesis and synthetic construction.

The Cas endonuclease gene may be a plant codon optimized Streptococcus pyogenes Cas9 gene that can recognize any genomic sequence of the form N(12-30)NGG can in principle be targeted.

The Cas endonuclease may be introduced directly into a cell by any method known in the art, for example, but not limited to transient introduction methods, transfection and/or topical application, including those described in US publication number 20160208272, published Jul. 26, 2016, and incorporated herein by reference. The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

As used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). A guide polynucleotide that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In one embodiment, the size of the fragment of the cRNA naturally occurring in Bacteria and Archaea that is present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In one example, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one example, the RNA that guides the RNA/Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some examples, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide RNA may comprise a cRNA or cRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some examples, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one example, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another example, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

The guide RNA and Cas endonuclease are capable of forming a complex that enables the Cas endonuclease to introduce in the plant genome a double strand break at a DNA target site, for example, in a male fertility gene locus of MS9, MS22, MS26 or MS45 or within MS9, MS22, MS26 or MS45 polynucleotides themselves. The variable target domain may be 12, 13, 14, 15, 16, 17, 18, 19, 20, 21, 22, 23, 24, 25, 26, 27, 28, 29 or 30 nucleotides in length.

In some approaches, the guide RNA comprises a cRNA (or cRNA fragment) and a tracrRNA (or tracrRNA fragment) of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site.

The guide RNA can be introduced into a plant or plant cell directly using any method known in the art such as, but not limited to, particle bombardment or topical applications, for example, as described in US publication number 20160208272, published Jul. 26, 2016, and incorporated herein by reference.

The guide RNA may be introduced indirectly by introducing a recombinant DNA molecule comprising the corresponding guide DNA sequence operably linked to a plant specific promoter that is capable of transcribing the guide RNA in said plant cell. The term “corresponding guide DNA” includes a DNA molecule that is identical to the RNA molecule but has a “T” substituted for each “U” of the RNA molecule. The guide RNA may be introduced via particle bombardment or Agrobacterium transformation of a recombinant DNA construct comprising the corresponding guide DNA operably linked to a plant U6 polymerase III promoter. Some embodiments of the disclosure relate to identified wheat U6 RNA polymerase III promoters, for example, wheat U6 RNA polymerase III promoter (SEQ ID NO: 451), wheat U6 RNA polymerase Ill promoter (SEQ ID NO: 452), and wheat U6 RNA polymerase III promoter (SEQ ID NO: 453). See, for example, Example 4 as described herein.

The RNA that guides the RNA/Cas9 endonuclease complex, may be a duplexed RNA comprising a duplex crRNA-tracrRNA. One advantage of using a guide RNA versus a duplexed crRNA-tracrRNA is that only one expression cassette needs to be made to express the fused guide RNA.

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

The target site may be similar to a DNA recognition site or target site that that is specifically recognized and/or bound by a double-strand break inducing agent such as a LIG3-4 endonuclease (US patent publication 2009-0133152 A1 (published May 21, 2009) or a MS26++ meganuclease (U.S. patent publication 20150184194 published Jul. 2, 2015).

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii). For example, the methods and compositions described herein may be used to produce a Ms9, Ms22, Ms26, or Ms45 modified target site which confers male-sterility to the plant containing the modified Ms9, Ms22, Ms26, or Ms45 target site or introduced genetic modification.

Methods for modifying a plant genomic target site are disclosed herein.

In another embodiment, the method includes modifying a target site located in or near a MS9, MS22, MS26, or MS45 gene in the genome of a plant cell, the method comprising introducing a guide RNA into a plant cell having a Cas endonuclease, wherein said guide RNA and Cas endonuclease are capable of forming a complex that enables the Cas endonuclease to introduce a double strand break at said target site.

Also provided is a method for modifying a target site in the genome of a plant cell, the method comprising introducing a guide RNA and a Cas endonuclease into said plant, wherein said guide RNA and Cas endonuclease are capable of forming a complex that enables the Cas endonuclease to introduce a double strand break at said target site. In some embodiments, the guideRNA can simultaneously modify the same target site in multiple genomes in the plant cell or plant. See, for example, Example 2, demonstrating the generation of ms45 mutations in the a-, b- and d-genomes in wheat using the same guideRNA. Table 2 provided herein shows exemplary wheat guideRNAs and target sequences for making Ms9, MS22, Ms26, or Ms45 mutations in wheat, maize, or rice genomes to confer male-sterility to a plant. Additionally, the target sequences listed for wheat are consensus sequences so that each genome can be modified simultaneously using the same guideRNA to produce the genetic modification.

Further provided is a method for modifying a target site in or near a MS9, MS22, MS26, or MS45 gene in the genome of a plant cell, the method comprising: a) introducing into a plant cell a guide RNA comprising a variable targeting domain and a Cas endonuclease, wherein said guide RNA and Cas endonuclease are capable of forming a complex that enables the Cas endonuclease to introduce a double strand break at said target site; and, b) identifying at least one plant cell that has a modification at said target, wherein the modification includes at least one deletion or substitution of one or more nucleotides in said target site. A plant derived the modified plant cell is male-sterile.

Further provided, a method for modifying a target DNA sequence in or near a MS9, MS22, MS26, or MS45 gene in the genome of a plant cell, the method comprising: a) introducing into a plant cell a first recombinant DNA construct capable of expressing a guide RNA and a second recombinant DNA construct capable of expressing a Cas endonuclease, wherein said guide RNA and Cas endonuclease are capable of forming a complex that enables the Cas endonuclease to introduce a double strand break at said target site; and, b) identifying at least one plant cell that has a modification at said target, wherein the modification includes at least one deletion or substitution of one or more nucleotides in said target site and the modification confers male-sterility to a plant derived from the modified plant cell.

The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs.

In some embodiment, the genomic target site capable of being cleaved by a Cas endonuclease comprises a 12 to 30 nucleotide fragment of a male fertility gene. Exemplary male fertility genes for use in the compositions and methods described here include but are not limited to MS9, MS22, MS26, and MS45. In some embodiments, the MS9, MS22, MS26, and MS45 fertility genes or gene loci to be targeted are from wheat, barley, maize, rice, sorghum, rye, millet, oats, sugarcane, turfgrass, switchgrass, soybean, canola, alfalfa, sunflower, cotton, tobacco, peanut, potato, tobacco, Arabidopsis, or safflower.

TABLE 2
Exemplary Guide RNAs and Target Sequence Description

| | | | | Start and | | | |
|---|---|---|---|---|---|---|---|
| | | | | End | | DNA | |
| Target | Target Sequence | Start | End | Positions | | version of | |
| Sequence | (5′-3′) and | Position | Position | of Target | PAM | Guide | Target |
| Designation | target strand | (bp) | (bp) | Sequence | Sequence | RNA | Site |
| Wheat Ms9 | GCGGCCGCCGTGCTGCG | 6 | 28 | Positions | AGG | GCGGCCGC | Exon1 |
| 4BL CR1 | ACAAGG - | | | relative to | | CGTGCTGC | |
| | Sense | | | ATG in SEQ | | GACA | |
| | (SEQ ID NO: 81) | | | ID NO: 5 | | (SEQ ID | |
| | | | | | | NO: 267) | |
| Wheat Ms9 | GCGTCCTCCTCCGCCGTC | 46 | 68 | Positions | CGG | GCGTCCTC | Exon1 |
| 4BL CR2 | CACGG - | | | relative to | | CTCCGCC | |
| | Complementary | | | ATG in SEQ | | GTCCA | |
| | (SEQ ID NO: 82) | | | ID NO: 5 | | (SEQ ID | |
| | | | | | | NO: 268) | |
| Wheat Ms9 | GAAGAAGGGGCCGTGG | 36 | 58 | Positions | CGG | GAAGAAG | Exon1 |
| 4BL CR3 | ACGGCGG -Sense | | | relative to | | GGGCCGT | |
| | (SEQ ID NO: 83) | | | ATG in SEQ | | GGACGG | |
| | | | | ID NO: 5 | | (SEQ ID | |
| | | | | | | NO: 269) | |
| Wheat Ms9 | GAAGGGGCCGTGGACG | 39 | 61 | Positions | AGG | GAAGGGG | Exon1 |
| 4BL CR4 | GCGGAGG -Sense | | | relative to | | CCGTGGA | |
| | (SEQ ID NO: 84) | | | ATG in SEQ | | CGGCGG | |
| | | | | ID NO: 5 | | (SEQ ID | |
| | | | | | | NO: 270) | |
| Wheat Ms9 | GGGGCCGTGGACGGCG | 42 | 64 | Positions | AGG | GGGGCCG | Exon1 |
| 4BL CR5 | GAGGAGG -Sense | | | relative to | | TGGACGG | |
| | (SEQ ID NO: 85) | | | ATG in SEQ | | CGGAGG | |
| | | | | ID NO: 5 | | (SEQ ID | |
| | | | | | | NO: 271) | |
| Wheat Ms9 | GGTGCCATGGTTGGAGG | 80 | 102 | Positions | AGG | GGTGCCA | Exon1 |
| 4BL CR6 | TGTAGG - | | | relative to | | TGGTTGG | |
| | Complementary | | | ATG in SEQ | | AGGTGT | |
| | (SEQ ID NO: 86) | | | ID NO: 5 | | (SEQ ID | |
| | | | | | | NO: 272) | |
| Wheat Ms9 | GTTGCCGGTGCCATGGT | 86 | 108 | Positions | AGG | GTTGCCG | Exon1 |
| 4BL CR7 | TGGAGG - | | | relative to | | GTGCCAT | |
| | Complementary | | | ATG in SEQ | | GGTTGG | |
| | (SEQ ID NO: 87) | | | ID NO: 5 | | (SEQ ID | |
| | | | | | | NO: 273) | |
| Wheat Ms9 | GGGAACAGAGGTCCAGT | 101 | 123 | Positions | CGG | GGGAACA | Exon1 |
| 4BL CR8 | TGCCGG - | | | relative to | | GAGGTCC | |
| | Complementary | | | ATG in SEQ | | AGTTGC | |
| | (SEQ ID NO: 88) | | | ID NO: 5 | | (SEQ ID | |
| | | | | | | NO: 274 | |
| Wheat Ms9 | GCACGAGAACTTCACGC | 309 | 331 | Positions | AGG | GCACGAG | Exon2 |
| 4BL CR9 | AGGAGG -Sense | | | relative to | | AACTTCAC | |
| | (SEQ ID NO: 89) | | | ATG in SEQ | | GCAGG | |
| | | | | ID NO: 5 | | (SEQ ID | |
| | | | | | | NO: 275) | |
| Wheat Ms9 | GAACTTCACGCAGGAGG | 315 | 337 | Positions | AGG | GAACTTC | Exon2 |
| 4BL CR10 | AGGAGG -Sense | | | relative to | | ACGCAGG | |
| | (SEQ ID NO: 90) | | | ATG in SEQ | | AGGAGG | |
| | | | | ID NO: 5 | | (SEQ ID | |
| | | | | | | NO: 276) | |
| Wheat Ms9 | GCTTCCCAGCATGGCAT | 350 | 372 | Positions | GGG | GCTTCCCA | Exon2 |
| 4BL CR11 | GGAGGG - | | | relative to | | GCATGGC | |
| | Complementary | | | ATG in SEQ | | ATGGA | |
| | (SEQ ID NO: 91) | | | ID NO: 5 | | (SEQ ID | |
| | | | | | | NO: 277) | |
| Wheat Ms9 | GTCACCCTCCATGCCATG | 346 | 368 | Positions | GGG | GTCACCCT | Exon2 |
| 4BL CR12 | CTGGG -Sense | | | relative to | | CCATGCC | |
| | (SEQ ID NO: 92) | | | ATG in SEQ | | ATGCT | |
| | | | | ID NO: 5 | | (SEQ ID | |
| | | | | | | NO: 278 | |
| Wheat Ms9 | GAACCAGCTGCCGGGGC | 882 | 904 | Positions | CGG | GAACCAG | Exon3 |
| 4BL CR13 | GGACGG -Sense | | | relative to | | CTGCCGG | |
| | (SEQ ID NO: 93) | | | ATG in SEQ | | GGCGGA | |
| | | | | ID NO: 5 | | (SEQ ID | |
| | | | | | | NO: 279) | |
| Wheat Ms9 | TGAGGTACACCAACTAC | 275 | 297 | Positions | AGG | TGAGGTAC | Exon2 |
| 4BL CR14 | CTGAGG-Sense | | | relative to | | ACCAACTA | |
| | (SEQ ID NO: 94) | | | ATG in SEQ | | CCTG | |
| | | | | ID NO: 5 | | (SEQ ID | |
| | | | | | | NO: 280) | |
| Wheat Ms9 | GCCAGGGGCGGCGGCG | 1185 | 1207 | Positions | CGG | GCCAGGG | Exon3 |
| 4BL CR15 | TCCCCGG -Sense | | | relative to | | GCGGCGG | |
| | (SEQ ID NO: 95) | | | ATG in SEQ | | CGTCCC | |
| | | | | ID NO: 5 | | (SEQ ID | |
| | | | | | | NO: 281) | |
| Maize Ms9 | GGTGCCATGGGTGGAG | 80 | 102 | Positions | AGG | GGTGCCAT | Exon1 |
| CR1 | GTGTAGG- | | | relative to | | GGGTGGA | |
| | Complementary | | | ATG in SEQ | | GGTGT | |
| | (SEQ ID NO: 96) | | | ID NO: 13 | | (SEQ ID | |
| | | | | | | NO: 282) | |
| Maize Ms9 | GTTGCCGGTGCCATGGG | 86 | 108 | Positions | AGG | GTTGCCG | Exon1 |
| CR2 | TGGAGG- | | | relative to | | GTGCCAT | |
| | Complementary | | | ATG in SEQ | | GGGTGG | |
| | (SEQ ID NO: 97) | | | ID NO: 13 | | (SEQ ID | |
| | | | | | | NO: 283) | |
| Maize Ms9 | GGTCCAGTTGCCGGTGC | 92 | 114 | Positions | GGG | GGTCCAG | Exon1 |
| CR3 | CATGGG- | | | relative to | | TTGCCGG | |
| | Complementary | | | ATG in SEQ | | TGCCAT | |
| | (SEQ ID NO: 98) | | | ID NO: 13 | | (SEQ ID | |
| | | | | | | NO: 284) | |
| Maize Ms9 | GGGCACGTTGGTCCAGT | 101 | 123 | Positions | CGG | GGGCACG | Exon1 |
| CR4 | TGCCGG- | | | relative to | | TTGGTCCA | |
| | Complementary | | | ATG in SEQ | | GTTGC | |
| | (SEQ ID NO: 99) | | | ID NO: 13 | | (SEQ ID | |
| | | | | | | NO: 285) | |
| Maize Ms9 | GCGGCAAGAGCTGCAG | 244 | 266 | Positions | AGG | GCGGCAA | Exon2 |
| CR5 | GCTGAGG-Sense | | | relative to | | GAGCTGC | |
| | (SEQ ID NO: 100 | | | ATG in SEQ | | AGGCTG | |
| | | | | ID NO: 13 | | (SEQ ID | |
| | | | | | | NO: 286) | |
| Maize Ms9 | GAAGTTCTCGTGCTTCA | 286 | 308 | Positions | TGG | GAAGTTC | Exon2 |
| CR6 | GGTTGG- | | | relative to | | TCGTGCTT | |
| | Complementary | | | ATG in SEQ | | CAGGT | |
| | (SEQ ID NO: 101) | | | ID NO: 13 | | (SEQ ID | |
| | | | | | | NO: 287) | |
| Maize Ms9 | GGGTGAAGTTCTCGTGC | 290 | 312 | Positions | AGG | GGGTGAA | Exon2 |
| CR7 | TTCAGG- | | | relative to | | GTTCTCGT | |
| | Complementary | | | ATG in SEQ | | GCTTC | |
| | (SEQ ID NO: 102) | | | ID NO: 13 | | (SEQ ID | |
| | | | | | | NO: 288) | |
| Maize Ms9 | GATGAGGTCTTCCTCCTC | 310 | 332 | Positions | GGG | GATGAGG | Exon2 |
| CR8 | CTGGG-Complementary | | | relative to | | TCTTCCTC | |
| | (SEQ ID NO: 103) | | | ATG in SEQ | | CTCCT | |
| | | | | ID NO: 13 | | (SEQ ID | |
| | | | | | | NO: 289) | |
| Maize Ms9 | GAAGCACGAGAACTTCA | 293 | 315 | Positions | AGG | GAAGCAC | Exon2 |
| CR9 | CCCAGG-Sense | | | relative to | | GAGAACT | |
| | (SEQ ID NO: 104) | | | ATG in SEQ | | TCACCC | |
| | | | | ID NO: 13 | | (SEQ ID | |
| | | | | | | NO: 290) | |
| Maize Ms9 | GCACGAGAACTTCACCC | 296 | 315 | Positions | AGG | GCACGAG | Exon2 |
| CR10 | AGGAGG-Sense | | | relative to | | AACTTCAC | |
| | (SEQ ID NO: 105 | | | ATG in SEQ | | CCAGG | |
| | | | | ID NO: 13 | | (SEQ ID | |
| | | | | | | NO: 291) | |
| Maize Ms9 | GCTTCCGAGCATGGCGT | 337 | 359 | Positions | GGG | GCTTCCG | Exon2 |
| CR11 | GGAGGG- | | | relative to | | AGCATGG | |
| | Complementary | | | ATG in SEQ | | CGTGGA | |
| | (SEQ ID NO: 106) | | | ID NO: 13 | | (SEQ ID | |
| | | | | | | NO: 292) | |
| Maize Ms9 | GTCACCCTCCACGCCATG | 333 | 355 | Positions | CGG | GTCACCCT | Exon2 |
| CR12 | CTCGG-Sense | | | relative to | | CCACGCC | |
| | (SEQ ID NO: 107) | | | ATG in SEQ | | ATGCT | |
| | | | | ID NO: 13 | | (SEQ ID | |
| | | | | | | NO: 293) | |
| Maize Ms9 | GAACCAGCTGCCGGGAA | 1284 | 1306 | Positions | CGG | GAACCAG | Exon3 |
| CR13 | GGACGG-Sense | | | relative to | | CTGCCGG | |
| | (SEQ ID NO: 108) | | | ATG in SEQ | | GAAGGA | |
| | | | | ID NO: 13 | | (SEQ ID | |
| | | | | | | NO: 294) | |
| Maize Ms9 | GCAAGAAGCTGCGGCA | 1343 | 1365 | Positions | GGG | GCAAGAA | Exon3 |
| CR14 | GCGCGGG-Sense | | | relative to | | GCTGCGG | |
| | (SEQ ID NO: 109) | | | ATG in SEQ | | CAGCGC | |
| | | | | ID NO: 13 | | (SEQ ID | |
| | | | | | | NO: 295) | |
| Maize Ms9 | GCGATGGGGCGGTGGG | 1372 | 1394 | Positions | GGG | GCGATGG | Exon3 |
| CR15 | TGAGGGG- | | | relative to | | GGCGGTG | |
| | Complementary | | | ATG in SEQ | | GGTGAG | |
| | (SEQ ID NO: 110) | | | ID NO: 13 | | (SEQ ID | |
| | | | | | | NO: 296) | |
| Rice Ms9 | GCTGCGACAAGGCGAAC | 17 | 43 | Positions | AGG | GCTGCGA | Exon1 |
| CR1 | GTGAAGAAGG-Sense | | | relative to | | CAAGGCG | |
| | (SEQ ID NO: 111) | | | ATG in SEQ | | AACGTGA | |
| | | | | ID NO: 17 | | AGA | |
| | | | | | | (SEQ ID | |
| | | | | | | NO: 297 | |
| Rice Ms9 | GCTGCGACAAGGCGAAC | 17 | 44 | Positions | GGG | GCTGCGAC | Exon1 |
| CR2 | GTGAAGAAGGG-Sense | | | relative to | | AAGGCGAA | |
| | (SEQ ID NO: 112) | | | ATG in SEQ | | CGTGAAGA | |
| | | | | ID NO: 17 | | A | |
| | | | | | | (SEQ ID | |
| | | | | | | NO: 298) | |
| Rice Ms9 | GGCGTACACCTCCACCC | 78 | 104 | Positions | CGG | GGCGTACA | Exon1 |
| CR3 | ACGGCACCGG-Sense | | | relative to | | CCTCCACCC | |
| | (SEQ ID NO: 113) | | | ATG in SEQ | | ACGGCAC | |
| | | | | ID NO: 17 | | (SEQ ID | |
| | | | | | | NO: 299) | |
| Rice Ms9 | GCGCCGGCGCCGCAGGC | 1981 | 2006 | Positions | CGG | GCGCCGGC | Exon3 |
| CR4 | TGCTCTCGG-Sense | | | relative to | | GCCGCAGG | |
| | (SEQ ID NO: 114) | | | ATG in SEQ | | CTGCTCT | |
| | | | | ID NO: 17 | | (SEQ ID | |
| | | | | | | NO: 300) | |
| Rice Ms9 | GCAGGCTGCTCTCGGCC | 1992 | 2017 | Positions | AGG | GCAGGCTG | Exon3 |
| CR5 | AGTATCAGG-Sense | | | relative to | | CTCTCGGC | |
| | (SEQ ID NO: 115) | | | ATG in SEQ | | CAGTATC | |
| | | | | ID NO: 17 | | (SEQ ID | |
| | | | | | | NO: 301) | |
| Rice Ms9 | GGCTGAGGTACACCAAC | 339 | 364 | Positions | AGG | GGCTGAGG | Exon2 |
| CR6 | TACCTGAGG-Sense | | | relative to | | TACACCAA | |
| | (SEQ ID NO: 116) | | | ATG in SEQ | | CTACCTG | |
| | | | | ID NO: 17 | | (SEQ ID | |
| | | | | | | NO: 302) | |
| Rice Ms9 | GAAGCACGAGAACTTCA | 373 | 395 | Positions | AGG | GAAGCACG | Exon2 |
| CR7 | CGCAGG-Sense | | | relative to | | AGAACTTC | |
| | (SEQ ID NO: 117) | | | ATG in SEQ | | ACGC | |
| | | | | ID NO: 17 | | (SEQ ID | |
| | | | | | | NO: 303) | |
| Rice Ms9 | GAAGCACGAGAACTTCA | 373 | 398 | Positions | AGG | GAAGCACG | Exon2 |
| CR8 | CGCAGGAGG-Sense | | | relative to | | AGAACTTC | |
| | (SEQ ID NO: 118) | | | ATG in SEQ | | ACGCAGG | |
| | | | | ID NO: 17 | | (SEQ ID | |
| | | | | | | NO: 304) | |
| Rice Ms9 | GCACGAGAACTTCACGC | 376 | 401 | Positions | AGG | GCACGAGA | Exon2 |
| CR9 | AGGAGGAGG-Sense | | | relative to | | ACTTCACG | |
| | (SEQ ID NO: 119) | | | ATG in SEQ | | CAGGAGG | |
| | | | | ID NO: 17 | | (SEQ ID | |
| | | | | | | NO: 305) | |
| Rice Ms9 | GTCACCCTCCACGCCATG | 413 | 434 | Positions | TGG | GTCACCCT | Exon2 |
| CR10 | CTGG-Sense | | | relative to | | CCACGCCA | |
| | (SEQ ID NO: 120) | | | ATG in SEQ | | TGC | |
| | | | | ID NO: 17 | | (SEQ ID | |
| | | | | | | NO: 306) | |
| Rice Ms9 | GTCACCCTCCACGCCATG | 413 | 435 | Positions | GGG | GTCACCCT | Exon2 |
| CR11 | CTGGG-Sense | | | relative to | | CCACGCCA | |
| | (SEQ ID NO: 121) | | | ATG in SEQ | | TGCT | |
| | | | | ID NO: 17 | | (SEQ ID | |
| | | | | | | NO: 307) | |
| Rice Ms9 | GGCTGCTCTCGGCCAGT | 1995 | 2020 | Positions | AGG | GGCTGCTC | Exon3 |
| CR12 | ATCAGGAGG -Sense | | | relative to | | TCGGCCAG | |
| | (SEQ ID NO: 122) | | | ATG in SEQ | | TATCAGG | |
| | | | | ID NO: 17 | | (SEQ ID | |
| | | | | | | NO: 308) | |
| Rice Ms9 | GGTCGCTGATCGCGAAC | 1613 | 1640 | Positions | CGG | GGTCGCTG | Exon3 |
| CR13 | CAGCTGCCGG-Sense | | | relative to | | ATCGCGAA | |
| | (SEQ ID NO: 123) | | | ATG in SEQ | | CCAGCTGC | |
| | | | | ID NO: 17 | | (SEQ ID | |
| | | | | | | NO: 309) | |
| Rice Ms9 | GACTCGCAGCATCTGCA | 1912 | 1938 | Positions | TGG | GACTCGCA | Exon3 |
| CR14 | GCTCAACTGG-Sense | | | relative to | | GCATCTGC | |
| | (SEQ ID NO: 124) | | | ATG in SEQ | | AGCTCAAC | |
| | | | | ID NO: 17 | | (SEQ ID | |
| | | | | | | NO: 310) | |
| Rice Ms9 | GCCGACCTCATGCAGAG | 1735 | 1757 | Positions | CGG | GCCGACCT | Exon3 |
| CR15 | CATCGG-Sense | | | relative to | | CATGCAGA | |
| | (SEQ ID NO: 125) | | | ATG in SEQ | | GCAT | |
| | | | | ID NO: 17 | | (SEQ ID | |
| | | | | | | NO: 311) | |
| Wheat | GTTGAGGATGCAGCAGC | 3 | 25 | Positions | TGG | GTTGAGG | Exon1 |
| Ms22CR1 | AGGTGG-Sense | | | relative to | | ATGCAGC | |
| | (SEQ ID NO: 126) | | | ATG in SEQ | | AGCAGG | |
| | | | | ID NO: 21 | | (SEQ ID | |
| | | | | | | NO: 312) | |
| Wheat | GAGGATGCAGCAGCAG | 6 | 28 | Positions | AGG | GAGGATG | Exon1 |
| Ms22CR2 | GTGGAGG-Sense | | | relative to | | CAGCAGC | |
| | (SEQ ID NO: 127) | | | ATG in SEQ | | AGGTGG | |
| | | | | ID NO: 21 | | (SEQ ID | |
| | | | | | | NO: 313) | |
| Wheat | GCAGCAGCAGGTGGAG | 12 | 34 | Positions | TGG | GCAGCAG | Exon1 |
| Ms22CR3 | GGCGTGG-Sense | | | relative to | | CAGGTGG | |
| | (SEQ ID NO: 128) | | | ATG in SEQ | | AGGGCG | |
| | | | | ID NO: 21 | | (SEQ ID | |
| | | | | | | NO: 314) | |
| Wheat | GCAGCAGGTGGAGGGC | 15 | 37 | Positions | TGG | GCAGCAG | Exon1 |
| Ms22CR4 | GTGGTGG-Sense | | | relative to | | GTGGAGG | |
| | (SEQ ID NO: 129) | | | ATG in SEQ | | GCGTGG | |
| | | | | ID NO: 21 | | (SEQ ID | |
| | | | | | | NO: 315) | |
| Wheat | GTGGAGGGCGTGGTGG | 22 | 44 | Positions | CGG | GTGGAGG | Exon1 |
| Ms22CR5 | GCGGCGG-Sense | | | relative to | | GCGTGGT | |
| | (SEQ ID NO: 130) | | | ATG in SEQ | | GGGCGG | |
| | | | | ID NO: 21 | | (SEQ ID | |
| | | | | | | NO: 316) | |
| Wheat | TGG-Complementary | 128 | 150 | Positions | TGG | GTGGCAC | Exon1 |
| Ms22CR6 | (SEQ ID NO: 131) | | | relative to | | ATGCAGC | |
| | | | | ATG in SEQ | | AGCCGC | |
| | | | | ID NO: 21 | | (SEQ ID | |
| | | | | | | NO: 317) | |
| Wheat | GTGGTCGTCTTCAGCGC | 112 | 134 | Positions | CGG | GTGGTCG | Exon1 |
| Ms22CR7 | CAGCGG-Sense | | | relative to | | TCTTCAGC | |
| | (SEQ ID NO: 132) | | | ATG in SEQ | | GCCAG | |
| | | | | ID NO: 21 | | (SEQ ID | |
| | | | | | | NO: 318) | |
| Wheat | GCAGGAGGCGCTTGACG | 147 | 169 | Positions | TGG | GCAGGAG | Exon1 |
| Ms22CR8 | ACGTGG- | | | relative to | | GCGCTTG | |
| | Complementary | | | ATG in SEQ | | ACGACG | |
| | (SEQ ID NO: 133) | | | ID NO: 21 | | (SEQ ID | |
| | | | | | | NO: 319) | |
| Wheat | GTCGTCAAGCGCCTCCT | 151 | 173 | Positions | TGG | GTCGTCA | Exon1 |
| Ms22CR9 | GCTTGG-Sense | | | relative to | | AGCGCCT | |
| | (SEQ ID NO: 134) | | | ATG in SEQ | | CCTGCT | |
| | | | | ID NO: 21 | | (SEQ ID | |
| | | | | | | NO: 320) | |
| Wheat | GTGGCCGGCGCCGGGTC | 260 | 282 | Positions | GGG | GTGGCCG | Exon1 |
| Ms22CR10 | CGGGGG- | | | relative to | | GCGCCGG | |
| | Complementary | | | ATG in SEQ | | GTCCGG | |
| | (SEQ ID NO: 135) | | | ID NO: 21 | | (SEQ ID | |
| | | | | | | NO: 321) | |
| Wheat | GGTGGCCGGCGCCGGG | 261 | 283 | Positions | GGG | GGTGGCC | Exon1 |
| Ms22CR11 | TCCGGGG- | | | relative to | | GGCGCCG | |
| | Complementary | | | ATG in SEQ | | GGTCCG | |
| | (SEQ ID NO: 136) | | | ID NO: 21 | | (SEQ ID | |
| | | | | | | NO: 322) | |
| Wheat | GTGGTGGCCGGCGCCG | 263 | 285 | Positions | CGG | GTGGTGG | Exon1 |
| Ms22CR12 | GGTCCGG- | | | relative to | | CCGGCGC | |
| | Complementary | | | ATG in SEQ | | CGGGTC | |
| | (SEQ ID NO: 137) | | | ID NO: 21 | | (SEQ ID | |
| | | | | | | NO: 323) | |
| Wheat | GGACGAGCGTGCCGTTG | 357 | 379 | Positions | TGG | GGACGAG | Exon1 |
| Ms22CR13 | ATGTGG- | | | relative to | | CGTGCCG | |
| | Complementary | | | ATG in SEQ | | TTGATG | |
| | (SEQ ID NO: 138) | | | ID NO: 21 | | (SEQ ID | |
| | | | | | | NO: 324) | |
| Wheat | GTGATGGCGTGCCACAT | 346 | 368 | Positions | CGG | GTGATGG | Exon1 |
| Ms22CR14 | CAACGG-Sense | | | relative to | | CGTGCCA | |
| | (SEQ ID NO: 139) | | | ATG in SEQ | | CATCAA | |
| | | | | ID NO: 21 | | (SEQ ID | |
| | | | | | | NO: 325) | |
| Wheat | GCGCCGGCGTCCTTGAG | 379 | 402 | Positions | CGG | GCGCCGG | Exon1 |
| Ms22CR15 | GAGCGG- | | | relative to | | CGTCCTTG | |
| | Complementary | | | ATG in SEQ | | AGGAG | |
| | (SEQ ID NO: 140) | | | ID NO: 21 | | (SEQ ID | |
| | | | | | | NO: 326) | |
| Maize Ms22 | GTCGGGAGTGAGCGGC | 36 | 58 | Positions | TGG | GTCGGGA | Exon1 |
| CR1 | GGCGTGG-Sense | | | relative to | | GTGAGCG | |
| | (SEQ ID NO: 141) | | | ATG in SEQ | | GCGGCG | |
| | | | | ID NO: 33 | | (SEQ ID | |
| | | | | | | NO: 327) | |
| Maize Ms22 | GGGAGTGAGCGGCGGC | 39 | 61 | Positions | TGG | GGGAGTG | Exon1 |
| CR2 | GTGGTGG-Sense | | | relative to | | AGCGGCG | |
| | (SEQ ID NO: 142) | | | ATG in SEQ | | GCGTGG | |
| | | | | ID NO: 33 | | (SEQ ID | |
| | | | | | | NO: 328) | |
| Maize Ms22 | GCGGACGCGGCGGCGG | 61 | 83 | Positions | CGG | GCGGACG | Exon1 |
| CR3 | CATCCGG-Sense | | | relative to | | CGGCGGC | |
| | (SEQ ID NO: 143) | | | ATG in SEQ | | GGCATC | |
| | | | | ID NO: 33 | | (SEQ ID | |
| | | | | | | NO: 329) | |
| Maize Ms22 | GGACGCGGCGGCGGCA | 63 | 85 | Positions | GGG | GGACGCG | Exon1 |
| CR4 | TCCGGGG-Sense | | | relative to | | GCGGCGG | |
| | (SEQ ID NO: 144) | | | ATG in SEQ | | CATCCG | |
| | | | | ID NO: 33 | | (SEQ ID | |
| | | | | | | NO: 330) | |
| Maize Ms22 | GCCACCATCGTCGTCGTC | 119 | 97 | Positions | CGG | GCCACCA | Exon1 |
| CR5 | GTCGG - | | | relative to | | TCGTCGTC | |
| | Complementary | | | ATG in SEQ | | GTCGT | |
| | (SEQ ID NO: 145) | | | ID NO: 33 | | (SEQ ID | |
| | | | | | | NO: 331 | |
| Maize Ms22 | GCCGACGACGACGACGA | 96 | 118 | Positions | TGG | GCCGACG | Exon1 |
| CR6 | TGGTGG-Sense | | | relative to | | ACGACGA | |
| | (SEQ ID NO: 146) | | | ATG in SEQ | | CGATGG | |
| | | | | ID NO: 33 | | (SEQ ID | |
| | | | | | | NO: 332) | |
| Maize Ms22 | GACGACGACGATGGTG | 102 | 124 | Positions | CGG | GACGACG | Exon1 |
| CR7 | GCCGCGG-Sense | | | relative to | | ACGATGG | |
| | (SEQ ID NO: 147) | | | ATG in SEQ | | TGGCCG | |
| | | | | ID NO: 33 | | (SEQ ID | |
| | | | | | | NO: 333) | |
| Maize Ms22 | GCCAGCGCCGACGCCGA | 149 | 127 | Positions | CGG | GCCAGCG | Exon1 |
| CR8 | GTGCGG- | | | relative to | | CCGACGC | |
| | Complementary | | | ATG in SEQ | | CGAGTG | |
| | (SEQ ID NO: 148) | | | ID NO: 33 | | (SEQ ID | |
| | | | | | | NO: 334) | |
| Maize Ms22 | GGTGGCCGCGGCGCCGC | 114 | 136 | Positions | CGG | GGTGGCC | Exon1 |
| CR9 | ACTCGG-Sense | | | relative to | | GCGGCGC | |
| | (SEQ ID NO: 149) | | | ATG in SEQ | | CGCACT | |
| | | | | ID NO: 33 | | (SEQ ID | |
| | | | | | | NO: 335) | |
| Maize Ms22 | GCCGCACTCGGCGTCGG | 126 | 148 | Positions | TGG | GCCGCAC | Exon1 |
| CR10 | CGCTGG-Sense | | | relative to | | TCGGCGT | |
| | (SEQ ID NO: 150) | | | ATG in SEQ | | CGGCGC | |
| | | | | ID NO: 33 | | (SEQ ID | |
| | | | | | | NO: 336) | |
| Maize Ms22 | GCACTCGGCGTCGGCGC | 129 | 151 | Positions | CGG | GCACTCG | Exon1 |
| CR11 | TGGCGG-Sense | | | relative to | | GCGTCGG | |
| | (SEQ ID NO: 151) | | | ATG in SEQ | | CGCTGG | |
| | | | | ID NO: 33 | | (SEQ ID | |
| | | | | | | NO: 337) | |
| Maize Ms22 | GGCGCTGGCGGTGTACG | 141 | 163 | Positions | GGG | GGCGCTG | Exon1 |
| CR12 | AGCGGG-Sense | | | relative to | | GCGGTGT | |
| | (SEQ ID NO: 152) | | | ATG in SEQ | | ACGAGC | |
| | | | | ID NO: 33 | | (SEQ ID | |
| | | | | | | NO: 338) | |
| Maize Ms22 | GCTGGCGGTGTACGAGC | 144 | 166 | Positions | TGG | GCTGGCG | Exon1 |
| CR13 | GGGTGG-Sense | | | relative to | | GTGTACG | |
| | (SEQ ID NO: 153) | | | ATG in SEQ | | AGCGGG | |
| | | | | ID NO: 33 | | (SEQ ID | |
| | | | | | | NO: 339) | |
| Maize Ms22 | GTACGAGCGGGTGGCG | 153 | 175 | Positions | TGG | GTACGAG | Exon1 |
| CR14 | CGCATGG-Sense | | | relative to | | CGGGTGG | |
| | (SEQ ID NO: 154) | | | ATG in SEQ | | CGCGCA | |
| | | | | ID NO: 33 | | (SEQ ID | |
| | | | | | | NO: 340) | |
| Maize Ms22 | GAGCGGGTGGCGCGCA | 157 | 179 | Positions | GGG | GAGCGGG | Exon1 |
| CR15 | TGGCGGG-Sense | | | relative to | | TGGCGCG | |
| | (SEQ ID NO: 155) | | | ATG in SEQ | | CATGGC | |
| | | | | ID NO: 33 | | (SEQ ID | |
| | | | | | | NO: 341) | |
| Rice Ms22 | GCGATGATGTCGGTGTA | 55 | 82 | Positions | TGG | GCGATGAT | Exon1 |
| CR1 | CGAGAGGGTGG-Sense | | | relative to | | GTCGGTGT | |
| | (SEQ ID NO: 156) | | | ATG in SEQ | | ACGAGAGG | |
| | | | | ID NO: 37 | | G | |
| | | | | | | (SEQ ID | |
| | | | | | | NO: 342) | |
| Rice Ms22 | GCGGTGGTGGTGTTCAG | 103 | 128 | Positions | CGG | GCGGTGGT | Exon1 |
| CR2 | CGCGAGCGG-Sense | | | relative to | | GGTGTTCA | |
| | (SEQ ID NO: 157) | | | ATG in SEQ | | GCGCGAG | |
| | | | | ID NO: 37 | | (SEQ ID | |
| | | | | | | NO: 343) | |
| Rice Ms22 | GCGGTGGTGGTGTTCAG | 103 | 129 | Positions | GGG | GCGGTGGT | Exon1 |
| CR3 | CGCGAGCGGG-Sense | | | relative to | | GGTGTTCA | |
| | (SEQ ID NO: 158) | | | ATG in SEQ | | GCGCGAGC | |
| | | | | ID NO: 37 | | (SEQ ID | |
| | | | | | | NO: 344) | |
| Rice Ms22 | GCTCGCCGCCGCCGCCG | 204 | 229 | Positions | AGG | GCTCGCCG | Exon1 |
| CR4 | ACATCCAGG-Sense | | | relative to | | CCGCCGCC | |
| | (SEQ ID NO: 159) | | | ATG in SEQ | | GACATCC | |
| | | | | ID NO: 37 | | (SEQ ID | |
| | | | | | | NO: 345) | |
| Rice Ms22 | GCGCTGTCGCAGCTCCT | 232 | 257 | Positions | GGG | GCGCTGTC | Exon1 |
| CR5 | CCCGCCGGG-Sense | | | relative to | | GCAGCTCC | |
| | (SEQ ID NO: 160) | | | ATG in SEQ | | TCCCGCC | |
| | | | | ID NO: 37 | | (SEQ ID | |
| | | | | | | NO: 346) | |
| Rice Ms22 | GTTCGTCGGCGGCAGGC | 279 | 305 | Positions | CGG | GTTCGTCG | Exon1 |
| CR6 | TCCTCGGCGG-Sense | | | relative to | | GCGGCAGG | |
| | (SEQ ID NO: 161) | | | ATG in SEQ | | CTCCTCGG | |
| | | | | ID NO: 37 | | (SEQ ID | |
| | | | | | | NO: 347) | |
| Rice Ms22 | GAAGGTGATGGCGTGCC | 312 | 338 | Positions | TGG | GAAGGTGA | Exon1 |
| CR7 | ACATCAATGG-Sense | | | relative to | | TGGCGTGC | |
| | ((SEQ ID NO: 162) | | | ATG in SEQ | | CACATCAA | |
| | | | | ID NO: 37 | | (SEQ ID | |
| | | | | | | NO: 348) | |
| Rice Ms22 | GGCACCCTCGTCCCCCTC | 337 | 364 | Positions | AGG | GGCACCCT | Exon1 |
| CR8 | CTCAAGCAGG-Sense | | | relative to | | CGTCCCCCT | |
| | (SEQ ID NO: 163)) | | | ATG in SEQ | | CCTCAAGC | |
| | | | | ID NO: 37 | | (SEQ ID | |
| | | | | | | NO: 349) | |
| Rice Ms22 | GTCCCCCTCCTCAAGCAG | 346 | 368 | Positions | CGG | GTCCCCCTC | Exon1 |
| CR9 | GCCGG-Sense | | | relative to | | CTCAAGCA | |
| | (SEQ ID NO: 164) | | | ATG in SEQ | | GGC | |
| | | | | ID NO: 37 | | (SEQ ID | |
| | | | | | | NO: 350) | |
| Rice Ms22 | GAGGGGGGTCAAAGGA | 1379 | 1398 | Positions | TGG | GAGGGGG | Exon1 |
| CR10 | GTGG-Complementary | | | in SEQ ID | | GTCAAAGG | |
| | (SEQ ID NO: 165) | | | NO: 38 | | AG | |
| | | | | | | (SEQ ID | |
| | | | | | | NO: 351) | |
| Rice Ms22 | GGTGCGAGGGGGGTCA | 1384 | 1403 | Positions | AGG | GGTGCGAG | Exon1 |
| CR11 | AAGG-Complementary | | | in SEQ ID | | GGGGGTCA | |
| | (SEQ ID NO: 166) | | | NO: 38 | | A | |
| | | | | | | (SEQ ID | |
| | | | | | | NO: 352) | |
| Rice Ms22 | GAATTGGTGTGGTGCGA | 1391 | 1413 | Positions | GGG | GAATTGGT | Exon1 |
| CR12 | GGGGGG- | | | in SEQ ID | | GTGGTGCG | |
| | Complementary | | | NO: 38 | | AGGG | |
| | (SEQ ID NO: 167) | | | | | (SEQ ID | |
| | | | | | | NO: 353) | |
| Rice Ms22 | GAATTGGTGTGGTGCGA | 1393 | 1413 | Positions | GGG | GAATTGGT | Exon1 |
| CR13 | GGGG-Complementary | | | in SEQ ID | | GTGGTGCG | |
| | (SEQ ID NO: 168) | | | NO: 38 | | AG | |
| | | | | | | (SEQ ID | |
| | | | | | | NO: 354) | |
| Rice Ms22 | GTCGAGCTCGTAGACGG | 181 | 201 | Positions | GGG | GTCGAGCT | Exon1 |
| CR14 | CGGG-Complementary | | | relative to | | CGTAGACG | |
| | (SEQ ID NO: 169) | | | ATG in SEQ | | GC | |
| | | | | ID NO: 37 | | (SEQ ID | |
| | | | | | | NO: 355) | |
| Rice Ms22 | GTCGAGCTCGTAGACGG | 182 | 201 | Positions | CGG | GTCGAGCT | Exon1 |
| CR15 | CGG-Complementary | | | relative to | | CGTAGACG | |
| | (SEQ ID NO: 170) | | | ATG in SEQ | | G | |
| | | | | ID NO: 37 | | (SEQ ID | |
| | | | | | | NO: 356) | |
| Wheat Ms26 | GGCAGGGCTCCACAAGT | 66 | 88 | Positions | TGG | GGCAGGG | Exon 1 |
| CR1 | TCATGG -Sense | | | relative to | | CTCCACAA | |
| | (SEQ ID NO: 171) | | | ATG in SEQ | | GTTCA | |
| | | | | ID NO: 41 | | (SEQ ID | |
| | | | | | | NO: 357) | |
| Wheat Ms26 | GCTTCCTCAGGCTCCACC | 123 | 145 | Positions | TGG | GCTTCCTC | Exon 1 |
| CR2 | AGTGG - | | | relative to | | AGGCTCC | |
| | Complementary | | | ATG in SEQ | | ACCAG | |
| | (SEQ ID NO: 172 | | | ID NO: 41 | | (SEQ ID | |
| | | | | | | NO: 358) | |
| Wheat Ms26 | GTGGAGCCTGAGGAAG | 129 | 151 | Positions | AGG | GTGGAGC | Exon 1 |
| CR3 | CAGAAGG -Sense | | | relative to | | CTGAGGA | |
| | (SEQ ID NO: 173) | | | ATG in SEQ | | AGCAGA | |
| | | | | ID NO: 41 | | (SEQ ID | |
| | | | | | | NO: 359) | |
| Wheat Ms26 | GGAGCCTGAGGAAGCA | 131 | 153 | Positions | GGG | GGAGCCT | Exon 1 |
| CR4 | GAAGGGG -Sense | | | relative to | | GAGGAAG | |
| | (SEQ ID NO: 174) | | | ATG in SEQ | | CAGAAG | |
| | | | | ID NO: 41 | | (SEQ ID | |
| | | | | | | NO: 360) | |
| Wheat Ms26 | GCCGGTCATCGGCGCGA | 165 | 187 | Positions | TGG | GCCGGTC | Exon 1 |
| CR5 | CGCTGG -Sense | | | relative to | | ATCGGCG | |
| | (SEQ ID NO: 175) | | | ATG in SEQ | | CGACGC | |
| | | | | ID NO: 41 | | (SEQ ID | |
| | | | | | | NO: 361) | |
| Wheat Ms26 | GCGCGACGCTGGAGCA | 176 | 198 | Positions | AGG | GCGCGAC | Exon 1 |
| CR6 | GCTGAGG -Sense | | | relative to | | GCTGGAG | |
| | (SEQ ID NO: 176) | | | ATG in SEQ | | CAGCTG | |
| | | | | ID NO: 41 | | (SEQ ID | |
| | | | | | | NO: 362) | |
| Wheat Ms26 | GTACCTGTCCAAGCACC | 231 | 253 | Positions | CGG | GTACCTGT | Exon 1 |
| CR7 | GGACGG -Sense | | | relative to | | CCAAGCA | |
| | (SEQ ID NO: 177) | | | ATG in SEQ | | CCGGA | |
| | | | | ID NO: 41 | | (SEQ ID | |
| | | | | | | NO: 363) | |
| Wheat Ms26 | GGAGGTGAAGGGCATG | 257 | 279 | Positions | CGG | GGAGGTG | Exon 1 |
| CR8 | TCGACGG - | | | relative to | | AAGGGCA | |
| | Complementary | | | ATG in SEQ | | TGTCGA | |
| | (SEQ ID NO: 178) | | | ID NO: 41 | | (SEQ ID | |
| | | | | | | NO: 364) | |
| Wheat Ms26 | GATGTAGGTGTAGGAG | 269 | 291 | Positions | AGG | GATGTAG | Exon 1 |
| CR9 | GTGAAGG - | | | relative to | | GTGTAGG | |
| | Complementary | | | ATG in SEQ | | AGGTGA | |
| | (SEQ ID NO: 179) | | | ID NO: 41 | | (SEQ ID | |
| | | | | | | NO: 365) | |
| Wheat Ms26 | GTCGGCGATGTAGGTGT | 275 | 297 | Positions | AGG | GTCGGCG | Exon 1 |
| CR10 | AGGAGG - | | | relative to | | ATGTAGG | |
| | Complementary | | | ATG in SEQ | | TGTAGG | |
| | (SEQ ID NO: 180) | | | ID NO: 41 | | (SEQ ID | |
| | | | | | | NO: 366) | |
| Wheat Ms26 | GTTCACCGGGTCGGCGA | 284 | 306 | Positions | AGG | GTTCACC | Exon 1 |
| CR11 | TGTAGG - | | | relative to | | GGGTCGG | |
| | Complementary | | | ATG in SEQ | | CGATGT | |
| | (SEQ ID NO: 181) | | | ID NO: 41 | | (SEQ ID | |
| | | | | | | NO: 367) | |
| Wheat Ms26 | GCCGAGCAGCACGTCCA | 456 | 478 | Positions | AGG | GCCGAGC | Exon 2 |
| CR12 | TGTAGG - | | | relative to | | AGCACGT | |
| | Complementary | | | ATG in SEQ | | CCATGT | |
| | (SEQ ID NO: 182) | | | ID NO: 41 | | (SEQ ID | |
| | | | | | | NO: 368) | |
| Wheat Ms26 | GGAGGTGTACAGGTCCT | 442 | 464 | Positions | TGG | GGAGGTG | Exon 2 |
| CR13 | ACATGG -Sense | | | relative to | | TACAGGT | |
| | (SEQ ID NO: 183) | | | ATG in SEQ | | CCTACA | |
| | | | | ID NO: 41 | | (SEQ ID | |
| | | | | | | NO: 369) | |
| Wheat Ms26 | GCGAGCTCTGGAGGAA | 501 | 523 | Positions | AGG | GCGAGCT | Exon 2 |
| CR14 | GCAGAGG -Sense | | | relative to | | CTGGAGG | |
| | (SEQ ID NO: 184) | | | ATG in SEQ | | AAGCAG | |
| | | | | ID NO: 41 | | (SEQ ID | |
| | | | | | | NO: 370) | |
| Wheat Ms26 | GCCTGCGCGAAGCTGTT | 821 | 843 | Positions | CGG | GCCTGCG | Exon 3 |
| CR15 | CTCCGG - | | | relative to | | CGAAGCT | |
| | Complementary | | | ATG in SEQ | | GTTCTC | |
| | (SEQ ID NO: 185) | | | ID NO: 41 | | (SEQ ID | |
| | | | | | | NO: 371) | |
| Maize Ms26 | GCGATGGCGTCGCCGGC | 15 | 37 | Positions | AGG | GCGATGG | Exon 1 |
| CR1 | GTGAGG - | | | relative to | | CGTCGCC | |
| | Complementary | | | ATG in SEQ | | GGCGTG | |
| | (SEQ ID NO: 186) | | | ID NO: 53 | | (SEQ ID | |
| | | | | | | NO: 372) | |
| Maize Ms26 | GGAGGAAGCTCACCTCA | 3 | 25 | Positions | CGG | GGAGGAA | Exon 1 |
| CR2 | CGCCGG -Sense | | | relative to | | GCTCACCT | |
| | (SEQ ID NO: 187) | | | ATG in SEQ | | CACGC | |
| | | | | ID NO: 53 | | (SEQ ID | |
| | | | | | | NO: 373) | |
| Maize Ms26 | GCTAGTGGGAAGAATG | 31 | 53 | Positions | TGG | GCTAGTG | Exon 1 |
| CR3 | GCGATGG - | | | relative to | | GGAAGAA | |
| | Complementary | | | ATG in SEQ | | TGGCGA | |
| | (SEQ ID NO: 188) | | | ID NO: 53 | | (SEQ ID | |
| | | | | | | NO: 374) | |
| Maize Ms26 | GGCCCTGCTAGTGGGAA | 37 | 59 | Positions | TGG | GGCCCTG | Exon 1 |
| CR4 | GAATGG - | | | relative to | | CTAGTGG | |
| | Complementary | | | ATG in SEQ | | GAAGAA | |
| | (SEQ ID NO: 189) | | | ID NO: 53 | | (SEQ ID | |
| | | | | | | NO: 375) | |
| Maize Ms26 | GGTTGTCCTCTCATGGAT | 84 | 106 | Positions | TGG | GGTTGTC | Exon 1 |
| CR5 | CCTGG -Sense | | | relative to | | CTCTCATG | |
| | (SEQ ID NO: 190) | | | ATG in SEQ | | GATCC | |
| | | | | ID NO: 53 | | (SEQ ID | |
| | | | | | | NO: 376) | |
| Maize Ms26 | GCTTCCTCAGGCTCCACC | 108 | 130 | Positions | TGG | GCTTCCTC | Exon 1 |
| CR6 | TCTGG -Complementary | | | relative to | | AGGCTCC | |
| | (SEQ ID NO: 191) | | | ATG in SEQ | | ACCTC | |
| | | | | ID NO: 53 | | (SEQ ID | |
| | | | | | | NO: 377) | |
| Maize Ms26 | GCCAGTCATCGGCGCAA | 150 | 172 | Positions | TGG | GCCAGTC | Exon 1 |
| CR7 | CGGTGG -Sense | | | relative to | | ATCGGCG | |
| | (SEQ ID NO: 192) | | | ATG in SEQ | | CAACGG | |
| | | | | ID NO: 53 | | (SEQ ID | |
| | | | | | | NO: 378) | |
| Maize Ms26 | GCGCAACGGTGGAGCA | 161 | 183 | Positions | AGG | GCGCAAC | Exon 1 |
| CR8 | GCTGAGG -Sense | | | relative to | | GGTGGAG | |
| | (SEQ ID NO: 193) | | | ATG in SEQ | | CAGCTG | |
| | | | | ID NO: 53 | | (SEQ ID | |
| | | | | | | NO: 379) | |
| Maize Ms26 | GGATGCACGACTGGCTT | 194 | 216 | Positions | GGG | GGATGCA | Exon 1 |
| CR9 | GTCGGG -Sense | | | relative to | | CGACTGG | |
| | (SEQ ID NO: 194) | | | ATG in SEQ | | CTTGTC | |
| | | | | ID NO: 53 | | (SEQ ID | |
| | | | | | | NO: 380) | |
| Maize Ms26 | GGCTTGTCGGGTACCTG | 206 | 228 | Positions | CGG | GGCTTGT | Exon 1 |
| CR10 | TCACGG -Sense | | | relative to | | CGGGTAC | |
| | (SEQ ID NO: 195) | | | ATG in SEQ | | CTGTCA | |
| | | | | ID NO: 53 | | (SEQ ID | |
| | | | | | | NO: 381) | |
| Maize Ms26 | GGAAGTGAACGGCATGT | 242 | 264 | Positions | CGG | GGAAGTG | Exon 1 |
| CR11 | CGACGG - | | | relative to | | AACGGCA | |
| | Complementary | | | ATG in SEQ | | TGTCGA | |
| | (SEQ ID NO: 196) | | | ID NO: 53 | | (SEQ ID | |
| | | | | | | NO: 382) | |
| Maize Ms26 | GGACATGCTCGACATTC | 282 | 304 | Positions | GGG | GGACATG | Exon 1 |
| CR12 | ACCGGG - | | | relative to | | CTCGACAT | |
| | Complementary | | | ATG in SEQ | | TCACC | |
| | (SEQ ID NO: 197) | | | ID NO: 53 | | (SEQ ID | |
| | | | | | | NO: 383) | |
| Maize Ms26 | GACGGCATCTTCAACGC | 455 | 477 | Positions | CGG | GACGGCA | Exon 2 |
| CR13 | CGACGG -Sense | | | relative to | | TCTTCAAC | |
| | (SEQ ID NO: 198) | | | ATG in SEQ | | GCCGA | |
| | | | | ID NO: 53 | | (SEQ ID | |
| | | | | | | NO: 384) | |
| Maize Ms26 | GCGAGCTGTGGAGGAA | 477 | 499 | Positions | AGG | GCGAGCT | Exon 2 |
| CR14 | GCAGAGG -Sense | | | relative to | | GTGGAGG | |
| | (SEQ ID NO: 199) | | | ATG in SEQ | | AAGCAG | |
| | | | | ID NO: 53 | | (SEQ ID | |
| | | | | | | NO: 385) | |
| Maize Ms26 | GTGGAGGAAGCAGAGG | 484 | 506 | Positions | CGG | GTGGAGG | Exon 2 |
| CR15 | AAGACGG -Sense | | | relative to | | AAGCAGA | |
| | (SEQ ID NO: 200) | | | ATG in SEQ | | GGAAGA | |
| | | | | ID NO: 53 | | (SEQ ID | |
| | | | | | | NO: 386) | |
| Maize Ms26 | GTACTCCATCCGCCCCAT | 1724 | 1747 | Positions | GGG | GTACTCCA | EXON 4 |
| CR16 | CGAGTAGGG - | | | relative to | | TCCGCCCC | |
| | Complementary | | | ATG in SEQ | | ATCGAGTA | |
| | (SEQ ID NO: 201) | | | ID NO: 53 | | (SEQ ID | |
| | | | | | | NO: 387) | |
| Maize Ms26 | GCACGTACGTCACCATC | 1698 | 1721 | Positions | CGG | GCACGTAC | EXON 4 |
| CR17 | CCGCCGG - | | | relative to | | GTCACCAT | |
| | Complementary | | | ATG in SEQ | | CCCGC | |
| | (SEQ ID NO: 202) | | | ID NO: 53 | | (SEQ ID | |
| | | | | | | NO: 388) | |
| Maize Ms26 | GACGTACGTGCCCTACT | 1711 | 1731 | Positions | GGG | GACGTACG | EXON 4 |
| CR18 | CGATGGG - | | | relative to | | TGCCCTACT | |
| | Sense | | | ATG in SEQ | | CGAT | |
| | (SEQ ID NO: 203) | | | ID NO: 53 | | (SEQ ID | |
| | | | | | | NO: 389) | |
| Rice Ms26 | GTGGATTCCTGCTACTG | 45 | 69 | Positions | GAAGAAT | GTGGATT | Exon 1 |
| CR1 | GGAAGAAT - | | | relative to | | CCTGCTAC | |
| | Complementary | | | ATG in SEQ | | TGG | |
| | (SEQ ID NO: 204) | | | ID NO: 57 | | (SEQ ID | |
| | | | | | | NO: 390) | |
| Rice Ms26 | GGCCTTGCATGCTTGGC | 599 | 622 | Positions | TCAGAAT | GGCCTTG | Exon 1 |
| CR2 | TCAGAAT - | | | relative to | | CATGCTTG | |
| | Complementary | | | ATG in SEQ | | GC | |
| | (SEQ ID NO: 205) | | | ID NO: 57 | | (SEQ ID | |
| | | | | | | NO: 391) | |
| Rice Ms26 | GTCCTGAAGACCAACTT | 307 | 388 | Positions | TACCCCA | GTCCTGA | Exon 1 |
| CR3 | CACCAATTACCCCAA - | | | relative to | A | AGACCAA | |
| | Sense | | | ATG in SEQ | | CTTCACCA | |
| | (SEQ ID NO: 206) | | | ID NO: 57 | | AT | |
| | | | | | | (SEQ ID | |
| | | | | | | NO: 392) | |
| Rice Ms26 | GGAAGACGGCGAGCTTC | 510 | 540 | Positions | GCCTCCA | GGAAGAC | Exon 2 |
| CR4 | GAGTTTGCCTCCAA - | | | relative to | A | GGCGAGC | |
| | Sense | | | ATG in SEQ | | TTCGAGTT | |
| | (SEQ ID NO: 207) | | | ID NO: 57 | | T | |
| | | | | | | (SEQ ID | |
| | | | | | | NO: 393) | |
| Rice Ms26 | GCCTCTACCCGGCGGTG | 1521 | 1551 | Positions | GACCCCA | GCCTCTAC | Exon 4 |
| CR5 | CCGCAGGACCCCAA - | | | relative to | A | CCGGCGG | |
| | Sense | | | ATG in SEQ | | TGCCGCA | |
| | (SEQ ID NO: 208) | | | ID NO: 57 | | G | |
| | | | | | | (SEQ ID | |
| | | | | | | NO: 394) | |
| Rice Ms26 | GACATCATTCTTCCCAGT | 39 | 69 | Positions | AATCCAC | GACATCA | Exon 1 |
| CR6 | AGCAGGAATCCAC - | | | relative to | | TTCTTCCC | |
| | Sense | | | ATG in SEQ | | AGTAGCA | |
| | (SEQ ID NO: 209) | | | ID NO: 57 | | GG | |
| | | | | | | (SEQ ID | |
| | | | | | | NO: 395) | |
| Rice Ms26 | GTTGTCCTCTCATGGATC | 91 | 117 | Positions | GGTCCAC | GTTGTCCT | Exon 1 |
| CR7 | TTGGTCCAC -Sense | | | relative to | | CTCATGG | |
| | (SEQ ID NO: 210) | | | ATG in SEQ | | ATCTT | |
| | | | | ID NO: 57 | | (SEQ ID | |
| | | | | | | NO: 396) | |
| Rice Ms26 | GCGCGACAGTGGAGCA | 167 | 198 | Positions | CTACCAC | GCGCGAC | Exon 1 |
| CR8 | ACTGAAGAACTACCAC - | | | relative to | | AGTGGAG | |
| | Sense | | | ATG in SEQ | | CAACTGA | |
| | (SEQ ID NO: 211) | | | ID NO: 57 | | AGAA | |
| | | | | | | (SEQ ID | |
| | | | | | | NO: 397) | |
| Rice Ms26 | GGCGGGATGGTGACGT | 1601 | 1632 | Positions | ACTCCAT | GGCGGGA | Exon 4 |
| CR9 | ACGTGCCCTACTCCAT - | | | relative to | | TGGTGAC | |
| | Sense | | | ATG in SEQ | | GTACGTG | |
| | (SEQ ID NO: 212) | | | ID NO: 57 | | CCCT | |
| | | | | | | (SEQ ID | |
| | | | | | | NO: 398) | |
| Rice Ms26 | GCCTACCTCCAGATGAA | 1784 | 1815 | Positions | TCGCCAT | GCCTACCT | Exon 4 |
| CR10 | GATGGCGCTCGCCAT - | | | relative to | | CCAGATG | |
| | Sense | | | ATG in SEQ | | AAGATGG | |
| | (SEQ ID NO: 213) | | | ID NO: 57 | | CGC | |
| | | | | | | (SEQ ID | |
| | | | | | | NO: 399) | |
| Rice Ms26 | GACCACCCCGTCAAGTA | 1849 | 1882 | Positions | TGACCAT | GACCACC | Exon 4 |
| CR11 | CCGGATGATGACCAT - | | | relative to | | CCGTCAA | |
| | Sense | | | ATG in SEQ | | GTACCGG | |
| | (SEQ ID NO: 214) | | | ID NO: 57 | | ATGA | |
| | | | | | | (SEQ ID | |
| | | | | | | NO: 400) | |
| Rice Ms26 | ACGTACGTGCCCTACTCC | 1613 | 1632 | Positions | | ACGTACGT | Exon 2 |
| CR12 | AT -Sense | | | relative to | | GCCCTACT | |
| | (SEQ ID NO: 215) | | | ATG in SEQ | | CCAT | |
| | | | | ID NO: 57 | | (SEQ ID | |
| | | | | | | NO: 401) | |
| Wheat Ms45 | GGGCCGCGATGAAGAG | 60 | 82 | Positions | GGG | GGGCCGC | Exon 1 |
| CR1 | GTGCGGG | | | relative to | | GATGAAG | |
| | Complementary | | | ATG in SEQ | | AGGTGC | |
| | (SEQ ID NO: 216) | | | ID NO: 61 | | (SEQ ID | |
| | | | | | | NO: 402) | |
| Wheat Ms45 | GCGCCAGGGCCGCGATG | 66 | 88 | Positions | AGG | GCGCCAG | Exon 1 |
| CR2 | AAGAGG - | | | relative to | | GGCCGCG | |
| | Complementary | | | ATG in SEQ | | ATGAAG | |
| | (SEQ ID NO: 217 | | | ID NO: 61 | | (SEQ ID | |
| | | | | | | NO: 403) | |
| Wheat Ms45 | GTACCCGCACCTCTTCAT | 57 | 79 | Positions | CGG | GTACCCG | Exon 1 |
| CR3 | CGCGG -Sense | | | relative to | | CACCTCTT | |
| | (SEQ ID NO: 218) | | | ATG in SEQ | | CATCG | |
| | | | | ID NO: 61 | | (SEQ ID | |
| | | | | | | NO: 404) | |
| Wheat Ms45 | GCACCTCTTCATCGCGGC | 63 | 85 | Positions | TGG | GCACCTCT | Exon 1 |
| CR4 | CCTGG -Sense | | | relative to | | TCATCGC | |
| | (SEQ ID NO: 219) | | | ATG in SEQ | | GGCCC | |
| | | | | ID NO: 61 | | (SEQ ID | |
| | | | | | | NO: 405) | |
| Wheat Ms45 | GCCAGCGGGCCGAGGT | 109 | 131 | Positions | GGG | GCCAGCG | Exon 1 |
| CR5 | GGAAGGG - | | | relative to | | GGCCGAG | |
| | Complementary | | | ATG in SEQ | | GTGGAA | |
| | (SEQ ID NO: 220) | | | ID NO: 61 | | (SEQ ID | |
| | | | | | | NO: 406) | |
| Wheat Ms45 | GGCGCCAGCTCGTGCTT | 148 | 170 | Positions | CGG | GGCGCCA | Exon 1 |
| CR6 | CACCGG - | | | relative to | | GCTCGTG | |
| | Complementary | | | ATG in SEQ | | CTTCAC | |
| | (SEQ ID NO: 221) | | | ID NO: 61 | | (SEQ ID | |
| | | | | | | NO: 407) | |
| Wheat Ms45 | GGTCATGCAGCGCTGGC | 180 | 202 | Positions | GGG | GGTCATG | Exon 1 |
| CR7 | CGAGGG -Sense | | | relative to | | CAGCGCT | |
| | (SEQ ID NO: 222) | | | ATG in SEQ | | GGCCGA | |
| | | | | ID NO: 61 | | (SEQ ID | |
| | | | | | | NO: 408) | |
| Wheat Ms45 | GCAGCGCTGGCCGAGG | 186 | 209 | Positions | CGG | GCAGCGC | Exon 1 |
| CR8 | GACAACGG- Sense | | | relative to | | TGGCCGA | |
| | (SEQ ID NO: 223) | | | ATG in SEQ | | GGGACAA | |
| | | | | ID NO: 61 | | (SEQ ID | |
| | | | | | | NO: 409 | |
| Wheat Ms45 | GGGACAACGGCAGCCG | 200 | 222 | Positions | AGG | GGGACAA | Exon 1 |
| CR9 | CCTCAGG -Sense | | | relative to | | CGGCAGC | |
| | (SEQ ID NO: 224) | | | ATG in SEQ | | CGCCTC | |
| | | | | ID NO: 61 | | (SEQ ID | |
| | | | | | | NO: 410) | |
| Wheat Ms45 | GCAGCCGCCTCAGGCTC | 209 | 231 | Positions | AGG | GCAGCCG | Exon 1 |
| CR10 | GGCAGG -Sense | | | relative to | | CCTCAGG | |
| | (SEQ ID NO: 225) | | | ATG in SEQ | | CTCGGC | |
| | | | | ID NO: 61 | | (SEQ ID | |
| | | | | | | NO: 411) | |
| Wheat Ms45 | GAGTTCGTCAACGAGGT | 235 | 257 | Positions | CGG | GAGTTCG | Exon 1 |
| CR11 | GTTCGG -Sense | | | relative to | | TCAACGA | |
| | (SEQ ID NO: 226) | | | ATG in SEQ | | GGTGTT | |
| | | | | ID NO: 61 | | (SEQ ID | |
| | | | | | | NO: 412) | |
| Wheat Ms45 | GACGCGGCCGTCGGCGA | 302 | 324 | Positions | CGG | GACGCGG | Exon 1 |
| CR12 | GCCCGG - | | | relative to | | CCGTCGG | |
| | Complementary | | | ATG in SEQ | | CGAGCC | |
| | (SEQ ID NO: 227) | | | ID NO: 61 | | (SEQ ID | |
| | | | | | | NO: 413) | |
| Wheat Ms45 | GGCCGCGTCGTGCGGTG | 316 | 338 | Positions | GGG | GGCCGCG | Exon 1 |
| CR13 | GATGGG -Sense | | | relative to | | TCGTGCG | |
| | (SEQ ID NO: 228) | | | ATG in SEQ | | GTGGAT | |
| | | | | ID NO: 61 | | (SEQ ID | |
| | | | | | | NO: 414) | |
| Wheat Ms45 | GCCGCGTCGTGCGGTGG | 317 | 339 | Positions | GGG | GCCGCGT | Exon 1 |
| CR14 | ATGGGG -Sense | | | relative to | | CGTGCGG | |
| | (SEQ ID NO: 229) | | | ATG in SEQ | | TGGATG | |
| | | | | ID NO: 61 | | (SEQ ID | |
| | | | | | | NO: 415) | |
| Wheat Ms45 | GACGGCGAACGTCTCCC | 347 | 369 | Positions | CGG | GACGGCG | Exon 1 |
| CR15 | ACCCGG - | | | relative to | | AACGTCTC | |
| | Complementary | | | ATG in SEQ | | CCACC | |
| | (SEQ ID NO: 230) | | | ID NO: 61 | | (SEQ ID | |
| | | | | | | NO: 416) | |
| Wheat Ms45 | GAAAGTTTGTGCTAACG | 466 | 488 | Positions | TGG | GAAAGTT | Exon 2 |
| CR16 | GAGTGG -Sense | | | relative to | | TGTGCTA | |
| | (SEQ ID NO: 231) | | | ATG in SEQ | | ACGGAG | |
| | | | | ID NO: 61 | | (SEQ ID | |
| | | | | | | NO: 417) | |
| Maize Ms45 | GGAACCTGCAGTGGCGG | 11 | 33 | Positions | GGG | GGAACCT | Exon 1 |
| CR1 | CGAGGG -Sense | | | relative to | | GCAGTGG | |
| | (SEQ ID NO: 232) | | | ATG in SEQ | | CGGCGA | |
| | | | | ID NO: 73 | | (SEQ ID | |
| | | | | | | NO: 418) | |
| Maize Ms45 | GGGCCGCGAAGAAGAG | 54 | 76 | Positions | GGG | GGGCCGC | Exon 1 |
| CR2 | GTGAGGG - | | | relative to | | GAAGAAG | |
| | Complementary | | | ATG in SEQ | | AGGTGA | |
| | (SEQ ID NO: 233) | | | ID NO: 73 | | (SEQ ID | |
| | | | | | | NO: 419) | |
| Maize Ms45 | GCGCCAGGGCCGCGAA | 60 | 82 | Positions | AGG | GCGCCAG | Exon 1 |
| CR3 | GAAGAGG - | | | relative to | | GGCCGCG | |
| | Complementary | | | ATG in SEQ | | AAGAAG | |
| | (SEQ ID NO: 234) | | | ID NO: 73 | | (SEQ ID | |
| | | | | | | NO: 420) | |
| Maize Ms45 | GTACCCTCACCTCTTCTT | 51 | 73 | Positions | CGG | GTACCCTC | Exon 1 |
| CR4 | CGCGG -Sense | | | relative to | | ACCTCTTC | |
| | (SEQ ID NO: 235) | | | ATG in SEQ | | TTCG | |
| | | | | ID NO: 73 | | (SEQ ID | |
| | | | | | | NO: 421) | |
| Maize Ms45 | GACTAGGAGGGCCAGC | 74 | 96 | Positions | GGG | GACTAGG | Exon 1 |
| CR5 | GCCAGGG - | | | relative to | | AGGGCCA | |
| | Complementary | | | ATG in SEQ | | GCGCCA | |
| | (SEQ ID NO: 236) | | | ID NO: 73 | | (SEQ ID | |
| | | | | | | NO: 422) | |
| Maize Ms45 | GAACGGGTCCGCGACTA | 86 | 108 | Positions | GGG | GAACGGG | Exon 1 |
| CR6 | GGAGGG - | | | relative to | | TCCGCGA | |
| | Complementary | | | ATG in SEQ | | CTAGGA | |
| | (SEQ ID NO: 237) | | | ID NO: 73 | | (SEQ ID | |
| | | | | | | NO: 423 | |
| Maize Ms45 | GGCCGAACGGGTCCGCG | 90 | 112 | Positions | AGG | GGCCGAA | Exon 1 |
| CR7 | ACTAGG - | | | relative to | | CGGGTCC | |
| | Complementary | | | ATG in SEQ | | GCGACT | |
| | (SEQ ID NO: 238) | | | ID NO: 73 | | (SEQ ID | |
| | | | | | | NO: 424) | |
| Maize Ms45 | GGCGCTGGCCCTCCTAG | 78 | 100 | Positions | CGG | GGCGCTG | Exon 1 |
| CR8 | TCGCGG -Sense | | | relative to | | GCCCTCCT | |
| | (SEQ ID NO: 239) | | | ATG in SEQ | | AGTCG | |
| | | | | ID NO: 73 | | (SEQ ID | |
| | | | | | | NO: 425) | |
| Maize Ms45 | GCCAGCGGACTGAGGCC | 103 | 125 | Positions | CGG | GCCAGCG | Exon 1 |
| CR9 | GAACGG - | | | relative to | | GACTGAG | |
| | Complementary | | | ATG in SEQ | | GCCGAA | |
| | (SEQ ID NO: 240) | | | ID NO: 73 | | (SEQ ID | |
| | | | | | | NO: 426) | |
| Maize Ms45 | GGCGCGAGCTCGTGCTT | 142 | 164 | Positions | CGG | GGCGCGA | Exon 1 |
| CR10 | CACCGG - | | | relative to | | GCTCGTG | |
| | Complementary | | | ATG in SEQ | | CTTCAC | |
| | (SEQ ID NO: 241) | | | ID NO: 73 | | (SEQ ID | |
| | | | | | | NO: 427) | |
| Maize Ms45 | GGCCGAGGTCGACTACC | 123 | 145 | Positions | CGG | GGCCGAG | Exon 1 |
| CR11 | GGCCGG -Sense | | | relative to | | GTCGACT | |
| | (SEQ ID NO: 242) | | | ATG in SEQ | | ACCGGC | |
| | | | | ID NO: 73 | | (SEQ ID | |
| | | | | | | NO: 428) | |
| Maize Ms45 | GCACGAGCTCGCGCCGT | 150 | 172 | Positions | GGG | GCACGAG | Exon 1 |
| CR12 | ACGGGG -Sense | | | relative to | | CTCGCGC | |
| | (SEQ ID NO: 243) | | | ATG in SEQ | | CGTACG | |
| | | | | ID NO: 73 | | (SEQ ID | |
| | | | | | | NO: 429) | |
| Maize Ms45 | GCGCCGTACGGGGAGG | 160 | 182 | Positions | GGG | GCGCCGT | Exon 1 |
| CR13 | TCATGGG -Sense | | | relative to | | ACGGGGA | |
| | (SEQ ID NO: 244) | | | ATG in SEQ | | GGTCAT | |
| | | | | ID NO: 73 | | (SEQ ID | |
| | | | | | | NO: 430) | |
| Maize Ms45 | GAGCCGGCTGGCATTGT | 191 | 213 | Positions | TGG | GAGCCGG | Exon 1 |
| CR14 | CTCTGG - | | | relative to | | CTGGCATT | |
| | Complementary | | | ATG in SEQ | | GTCTC | |
| | (SEQ ID NO: 245) | | | ID NO: 73 | | (SEQ ID | |
| | | | | | | NO: 431) | |
| Maize Ms45 | GGCCCAGAGACAATGCC | 188 | 210 | Positions | CGG | GGCCCAG | Exon 1 |
| CR15 | AGCCGG -Sense | | | relative to | | AGACAAT | |
| | (SEQ ID NO: 246) | | | ATG in SEQ | | GCCAGC | |
| | | | | ID NO: 73 | | (SEQ ID | |
| | | | | | | NO: 432) | |
| Maize Ms45 | GCTGGCCGAGGTCGACT | 120 | 141 | Positions | CGG | GCTGGCCG | Exon 1 |
| CR16 | ACCGG -Sense | | | relative to | | AGGTCGAC | |
| | (SEQ ID NO: 247) | | | ATG in SEQ | | TAC | |
| | | | | ID NO: 73 | | (SEQ ID | |
| | | | | | | NO: 433) | |
| Maize Ms45 | GGCCGAGGTCGACTACC | 123 | 142 | Positions | CGG | GGCCGAGG | Exon 1 |
| CR17 | GGCCGG -Sense | | | relative to | | TCGACTAC | |
| | (SEQ ID NO: 248) | | | ATG in SEQ | | CGGC | |
| | | | | ID NO: 73 | | (SEQ ID | |
| | | | | | | NO: 434) | |
| Maize Ms45 | GGCGCGAGCTCGTGCTT | 145 | 164 | Positions | CGG | GGCGCGAG | Exon 1 |
| CR18 | CACCGG - | | | relative to | | CTCGTGCTT | |
| | Complementary | | | ATG in SEQ | | CAC | |
| | (SEQ ID NO: 249) | | | ID NO: 73 | | (SEQ ID | |
| | | | | | | NO: 435) | |
| Rice Ms45 | GCGTACTACGGGCTCAT | 608 | 639 | Positions | GGTCCGA | GCGTACT | Exon 2 |
| CR1 | GTCCGTCGGTCCGAA - | | | relative to | A | ACGGGCT | |
| | Sense | | | ATG in SEQ | | CATGTCC | |
| | (SEQ ID NO: 250) | | | ID NO: 77 | | GTC | |
| | | | | | | (SEQ ID | |
| | | | | | | NO: 436) | |
| Rice Ms45 | GGGAGGCTGCTCAGATA | 894 | 926 | Positions | AAACCAA | GGGAGGC | Exon 3 |
| CR2 | TGACCCAGAAACCAAA - | | | relative to | A | TGCTCAG | |
| | Sense | | | ATG in SEQ | | ATATGAC | |
| | (SEQ ID NO: 251) | | | ID NO: 77 | | CCAG | |
| | | | | | | (SEQ ID | |
| | | | | | | NO: 437) | |
| Rice Ms45 | GTCGTGCTGAGCGGGCT | 936 | 964 | Positions | TTCCCGA | GTCGTGC | Exon 3 |
| CR3 | GGTCTTCCCGAA -Sense | | | relative to | A | TGAGCGG | |
| | (SEQ ID NO: 252) | | | ATG in SEQ | | GCTGGTC | |
| | | | | ID NO: 77 | | (SEQ ID | |
| | | | | | | NO: 438) | |
| Rice Ms45 | GCTGGCCCTGCTCCTCAC | 90 | 120 | Positions | GTTCCAC | GCTGGCC | Exon 1 |
| CR4 | CGACCCGTTCCAC - | | | relative to | | CTGCTCCT | |
| | Sense | | | ATG in SEQ | | CACCGAC | |
| | (SEQ ID NO: 253) | | | ID NO: 77 | | CC | |
| | | | | | | (SEQ ID | |
| | | | | | | NO: 439) | |
| Rice Ms45 | GTCGGAGAGGTGTTCGG | 244 | 272 | Positions | AGTCCAT | GTCGGAG | Exon 1 |
| CR5 | GCCGGAGTCCAT -Sense | | | relative to | | AGGTGTT | |
| | (SEQ ID NO: 254) | | | ATG in SEQ | | CGGGCCG | |
| | | | | ID NO: 77 | | G | |
| | | | | | | (SEQ ID | |
| | | | | | | NO: 440) | |
| Rice Ms45 | GTGCCTCACCGGCCGGT | 139 | 162 | Positions | AGTCCAC | GTGCCTC | Exon 1 |
| CR6 | AGTCCAC - | | | relative to | | ACCGGCC | |
| | Complementary | | | ATG in SEQ | | GGT | |
| | (SEQ ID NO: 255) | | | ID NO: 77 | | (SEQ ID | |
| | | | | | | NO: 441) | |
| Rice Ms45 | GCCGGCTGCCGTTGTCC | 195 | 220 | Positions | CGGCCAC | GCCGGCT | Exon 1 |
| CR7 | CGCGGCCAC - | | | relative to | | GCCGTTG | |
| | Complementary | | | ATG in SEQ | | TCCCG | |
| | (SEQ ID NO: 256) | | | ID NO: 77 | | (SEQ ID | |
| | | | | | | NO: 442) | |
| Rice Ms45 | GTCTCCCACCCGGCGTCC | 337 | 362 | Positions | CCCCCAT | GTCTCCCA | exon 1 |
| CR8 | TCCCCCAT - | | | relative to | | CCCGGCG | |
| | Complementary | | | ATG in SEQ | | TCCT | |
| | (SEQ ID NO: 257) | | | ID NO: 77 | | (SEQ ID | |
| | | | | | | NO: 443) | |
| Rice Ms45 | GCTCATGACGGCGAACG | 354 | 378 | Positions | CTCCCAC | GCTCATG | Exon 1 |
| CR9 | TCTCCCAC - | | | relative to | | ACGGCGA | |
| | Complementary | | | ATG in SEQ | | ACGT | |
| | (SEQ ID NO: 258) | | | ID NO: 77 | | (SEQ ID | |
| | | | | | | NO: 444) | |
| Rice Ms45 | GCTTCTTCGTCGTCGACT | 501 | 527 | Positions | ACCCCAT | GCTTCTTC | Exon 2 |
| CR10 | CCACCCCAT - | | | relative to | | GTCGTCG | |
| | Complementary | | | ATG in SEQ | | ACTCC | |
| | (SEQ ID NO: 259) | | | ID NO: 77 | | (SEQ ID | |
| | | | | | | NO: 445) | |
| Rice Ms45 | GACTTCTCTCGCGAGAG | 647 | 673 | Positions | TCGCCAC | GACTTCTC | Exon 2 |
| CR11 | AGGTCGCCAC - | | | relative to | | TCGCGAG | |
| | Complementary | | | ATG in SEQ | | AGAGG | |
| | (SEQ ID NO: 260) | | | ID NO: 77 | | (SEQ ID | |
| | | | | | | NO: 446) | |
| Rice Ms45 | GGTCGTCAGAAATCTGC | 964 | 987 | Positions | ACGCCAT | GGTCGTC | Exon 3 |
| CR12 | ACGCCAT - | | | relative to | | AGAAATC | |
| | Complementary | | | ATG in SEQ | | TGC | |
| | (SEQ ID NO: 261) | | | ID NO: 77 | | (SEQ ID | |
| | | | | | | NO: 447) | |
| Rice Ms45 | GAACCCCGGCAGGTCGG | 1157 | 1186 | Positions | CCTCCAC | GAACCCC | Exon 4 |
| CR13 | CGAACACCTCCAC - | | | relative to | | GGCAGGT | |
| | Complementary | | | ATG in SEQ | | CGGCGAA | |
| | (SEQ ID NO: 262) | | | ID NO: 77 | | CA | |
| | | | | | | (SEQ ID | |
| | | | | | | NO: 448) | |
| Rice Ms45 | GCTTGAAGTAGAGCGTT | 1288 | 1313 | Positions | CAGCCAC | GCTTGAA | Exon 4 |
| CR14 | CGCAGCCAC - | | | relative to | | GTAGAGC | |
| | Complementary | | | ATG in SEQ | | GTTCG | |
| | (SEQ ID NO: 263) | | | ID NO: 77 | | (SEQ ID | |
| | | | | | | NO: 449) | |
| Rice Ms45 | GGTGTGCATCCGCATGC | 1343 | 1366 | Positions | TGACCAT | GGTGTGC | Exon 4 |
| CR15 | TGACCAT - | | | relative to | | ATCCGCAT | |
| | Complementary | | | ATG in SEQ | | GC | |
| | (SEQ ID NO: 264) | | | ID NO: 77 | | (SEQ ID | |
| | | | | | | NO: 450) | |

Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease. Assays to measure the double-strand break of a target site by an endonuclease are known in the art and generally measure the overall activity and specificity of the agent on DNA substrates containing recognition sites.

As used herein, a “genomic region” is a segment of a chromosome in the genome of a plant cell. The genomic region may be present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932.

Alteration of the genome of a plant cell, for example, through homologous recombination (HR), is a powerful tool for genetic engineering. Despite the low frequency of homologous recombination in higher plants, there are a few examples of successful homologous recombination of plant endogenous genes. The parameters for homologous recombination in plants have primarily been investigated by rescuing introduced truncated selectable marker genes. In these experiments, the homologous DNA fragments were typically between 0.3 kb to 2 kb. Observed frequencies for homologous recombination were on the order of 10⁻⁴ to 10⁻⁵. See, for example, Halfter et al., (1992) Mol Gen Genet 231:186-93; Offringa et al., (1990) EMBO J 9:3077-84; Offringa et al., (1993) Proc. Natl. Acad. Sci. USA 90:7346-50; Paszkowski et al., (1988) EMBO J 7:4021-6; Hourda and Paszkowski, (1994) Mol Gen Genet 243:106-11; and Risseeuw et al., (1995) Plant J 7:109-19.

Once a double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9).

Alternatively, the double-strand break can be repaired by homologous recombination between homologous DNA sequences.

### Genome Editing Using the Guide RNA/Cas Endonuclease System

Further provided is a method for modifying a target site at or near a Ms9, Ms22, Ms26, or Ms45 gene in the genome of a plant cell, the method comprising introducing a guide RNA and a donor DNA into a plant cell having a Cas endonuclease, wherein said guide RNA and Cas endonuclease are capable of forming a complex that enables the Cas endonuclease to introduce a double strand break at said target site, wherein said donor DNA comprises a polynucleotide of interest that when inserted confers male-sterility to a plant obtained from the modified plant cell.

As described herein, the guide RNA/Cas endonuclease system can be used in combination with a co-delivered polynucleotide modification template to allow for editing of a genomic nucleotide sequence of interest, MS9, MS22, MS26, or MS45, to confer male-sterility to a plant. Also, as described herein, for each embodiment that uses a guide RNA/Cas endonuclease system, a similar guide polynucleotide/Cas endonuclease system can be deployed where the guide polynucleotide does not solely comprise ribonucleic acids but wherein the guide polynucleotide comprises a combination of RNA-DNA molecules or solely comprise DNA molecules.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In one embodiment provided herein, the method comprises contacting a plant cell with the donor DNA and the endonuclease. Once a double-strand break is introduced in the target site by the endonuclease, the first and second regions of homology of the donor DNA can undergo homologous recombination with their corresponding genomic regions of homology resulting in exchange of DNA between the donor and the genome. As such, the provided methods result in the integration of the polynucleotide of interest of the donor DNA into the double-strand break in the target site in the plant genome so that the endogenous male fertility gene of MS9, MS22, MS26, or MS45 is disrupted, thereby altering the original target site and producing an altered genomic target site that confers male sterility to the plant.

The donor DNA may be introduced by any means known in the art. For example, a plant having a target site is provided. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome to disrupt an endogenous male fertility gene of MS9, MS22, MS26, or MS45.

In one embodiment, the disclosure describes a method for editing a nucleotide sequence in the genome of a cell, the method comprising providing a guide RNA, a polynucleotide modification template, and at least one Cas endonuclease to a cell, wherein the Cas endonuclease is capable of introducing a double-strand break at a target sequence in the genome of said cell to confer male-sterility, wherein said polynucleotide modification template includes at least one nucleotide modification of said nucleotide sequence. The nucleotide to be edited can be located within or outside a target site of one or more MS9, MS22, MS26 or MS45 genes recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

In another embodiment of genome editing, editing of an endogenous MS9, MS22, MS26, and MS45 gene in a plant cell or plant is disclosed herein. In some embodiments, the polynucleotide modification template (male fertility gene polynucleotide modification template) includes a partial fragment of the MS9, MS22, MS26 or MS45 gene (and therefore does not encode a fully functional MS9, MS22, MS26 or MS45 polypeptide by itself).

In one embodiment of the disclosure, a wheat Ms9, Ms22, Ms26, or Ms45 mutant plant is produced by the method described herein, said method comprising: a) providing a guide RNA, a polynucleotide modification template and at least one Cas endonuclease to a plant cell, wherein the Cas endonuclease introduces a double strand break at a target site within a wheat Ms9, Ms22, Ms26, or Ms45 (male sterility 45) genomic sequence in the plant genome, wherein said polynucleotide modification template comprises at least one nucleotide modification of the Ms9, Ms22, Ms26, or Ms45 genomic sequence; b) obtaining a plant from the plant cell of (a); c) evaluating the plant of (b) for the presence of said at least one nucleotide modification and d) selecting a progeny plant exhibiting male sterility from the modification of the endogenous Ms9, Ms22, Ms26, or Ms45 gene. The nucleotide sequence to be edited may be a sequence that is endogenous to the cell that is being edited.

### Regulatory Sequence Modifications Using the Guide Polynucleotide/Cas Endonuclease System

In one example, the nucleotide sequence to be modified can be a regulatory sequence such as a promoter, for example, for an endogenous MS9, MS22, MS26, and MS45 gene in a plant cell or plant. In some examples, the promoter may be modified to include or remove an element in the promoter. In one embodiment, the guide polynucleotide/Cas endonuclease system can be used to allow for the deletion of a promoter or promoter element, wherein the promoter deletion (or promoter element deletion) results in any one of the following or any one combination of the following: a permanently inactivated gene locus, a decreased promoter activity, a decreased promoter tissue specificity, a modification of the timing or developmental progress of gene expression, a mutation of DNA binding elements and/or an addition of DNA binding elements. Promoter elements to be deleted can be, but are not limited to, promoter core elements, such as, but not limited to, a CAAT box, a CCAAT box, a Pribnow box, TATA box, and/or translational regulation sequences, promoter enhancer elements. The promoter or promoter fragment to be deleted may be endogenous to the cell that is being edited, for example, the promoter of an endogenous MS9, MS22, MS26, and MS45 fertility gene.

### Additional Regulatory Sequence Modifications Using the Guide Polynucleotide/Cas Endonuclease System

The guide polynucleotide/Cas endonuclease system may be used to modify or replace a regulatory sequence in the genome of a cell. A regulatory sequence is a segment of a nucleic acid molecule which is capable of increasing or decreasing the expression of specific genes within an organism and/or is capable of altering tissue specific expression of genes within an organism. Examples of regulatory sequences include, but are not limited to, 3′ UTR (untranslated region) region, 5′ UTR region, transcription activators, transcriptional enhancers transcriptions repressors, translational repressors, splicing factors, miRNAs, siRNA, artificial miRNAs, promoter elements, polyadenylation signals, and polyubiquitination sites. In one example, the editing (modification) or replacement of a regulatory element results in altered protein translation, RNA cleavage, RNA splicing, transcriptional termination or post translational modification that confers male-sterility to a plant. In one embodiment, regulatory elements can be identified within a promoter and these regulatory elements can be edited or modified do to optimize these regulatory elements for down regulation of the promoter to create a male sterile plant.

In one embodiment, the genomic sequence of interest to be modified is an intron or UTR site, wherein the modification consists of inserting at least one microRNA into said intron or UTR site, wherein expression of the gene comprising the intron or UTR site also results in expression of said microRNA, which in turn can silence any gene targeted by the microRNA without disrupting the gene expression of the native/transgene comprising said intron.

Modifications of Splicing Sites and/or Introducing Alternate Splicing Sites Using the Guide Polynucleotide/Cas Endonuclease System

The guide polynucleotide/Cas endonuclease system can be used in combination with a co-delivered polynucleotide modification template to edit an endogenous MS9, MS22, MS26, or MS45 gene to introduce a canonical splice site at a described junction or any variant of a splicing site that disrupts the splicing pattern of pre-mRNA molecules so that the plant with the introduced genetic modification is male-sterile.

### Modifications of Nucleotide Sequences Encoding a Protein of Interest Using the Guide Polynucleotide/Cas Endonuclease System

In one embodiment, the guide polynucleotide/Cas endonuclease system can be used to modify or replace a coding sequence in the fertility gene locus of MS9, MS22, MS26 or MS45 in the genome of a plant cell, wherein the modification or replacement results in conferring male-sterility to the plant. In one embodiment, the protein knockout is due to the introduction of a stop codon into the coding sequence of interest. In one embodiment, the protein knockout is due to the deletion of a start codon into the coding sequence of interest.

### Gene Silencing by Expressing an Inverted Repeat or Antisense Using the Guide Polynucleotide/Cas Endonuclease System

In one embodiment, the guide polynucleotide/Cas endonuclease system can be used in combination with a co-delivered polynucleotide sequence to insert an inverted gene fragment into a gene of interest in the genome of an organism, wherein the insertion of the inverted gene fragment can allow for an in-vivo creation of an inverted repeat (hairpin) and results in the silencing of said endogenous gene of MS9, MS22, MS26 or MS45, for example, a hairpin promoter inverted repeat (pIR) directed to MS9, MS22, MS26 or MS45.

In one embodiment, the insertion of the inverted gene fragment can result in the formation of an in-vivo created inverted repeat (hairpin) in a native (or modified) promoter of a gene and/or in a native 5′ end of the native gene. The inverted gene fragment can further comprise an intron which can result in an enhanced silencing of the targeted endogenous MS9, MS22, MS26 or MS45 gene.

In one embodiment, the region of interest can be flanked by two independent guide polynucleotide/CAS endonuclease target sequences. Cutting would be done concurrently. The deletion event would be the repair of the two chromosomal ends without the region of interest. Alternative results would include inversions of the region of interest, mutations at the cut sites and duplication of the region of interest. Furthermore, the introduced genetic modification may also comprise antisense sequences complementary to at least a portion of the messenger RNA (mRNA) for MS9, MS22, MS26 or MS45. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the introduced genetic modification may also be a polynucleotide arranged in the sense orientation to suppress the expression of endogenous MS9, MS22, MS26, or MS45 genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

Protocols for introducing polynucleotides and polypeptides into plants may vary depending on the type of plant or plant cell targeted for transformation, such as monocot or dicot. Suitable methods of introducing polynucleotides and polypeptides into plant cells include but are not limited to Agrobacterium-meditated transformation (U.S. Pat. Nos. 5,563,055 and 5,981,840), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin). Wheat transformation may be carried out by any suitable technique known to one skilled in the art, including those described in published patent application no. 20140173781 published on Jun. 19, 2014.

### Methods for Identifying at Least One Plant Cell Comprising in its Genome the Introduced Genetic Modification at the Target Site.

Further provided are methods for identifying at least one plant cell, comprising in its genome, the introduced genetic modification at the target site. A variety of methods are available for identifying those plant cells with the introduced genetic modification into the genome at or near to the target site without using a screenable marker phenotype. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference. The method also comprises recovering a male-sterile plant from the plant cell having the introduced genetic modification in its genome.

The present disclosure further provides expression constructs for expressing in a plant, plant cell, or plant part a guide RNA/Cas system that is capable of binding to and creating a double strand break in a target site of the fertility gene locus of MS9, MS22, MS26 or MS45. In one embodiment, the expression constructs of the disclosure comprise a promoter operably linked to a nucleotide sequence encoding a Cas gene and a promoter operably linked to a guide RNA of the present disclosure. The promoter is capable of driving expression of an operably linked nucleotide sequence in a plant cell.

A promoter is a region of DNA involved in recognition and binding of RNA polymerase and other proteins to initiate transcription. A plant promoter is a promoter capable of initiating transcription in a plant cell, for a review of plant promoters, see, Potenza et al., (2004) In Vitro Cell Dev Biol 40:1-22. Constitutive promoters include, for example, the core promoter of the Rsyn7 promoter and other constitutive promoters disclosed in WO99/43838 and U.S. Pat. No. 6,072,050; the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; Christensen et al., (1992) Plant Mol Biol 18:675-89); pEMU (Last et al., (1991) Theor Appl Genet 81:581-8); MAS (Velten et al., (1984) EMBO J 3:2723-30); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611. In some examples, an inducible promoter may be used. Pathogen-inducible promoters induced following infection by a pathogen include, but are not limited to those regulating expression of PR proteins, SAR proteins, beta-1,3-glucanase, chitinase, etc.

### Marker Assisted Selection and Breeding of Plants

Use of markers, and/or genetically-linked nucleic acids is an effective method for selecting plant having the desired traits in breeding programs. For example, one advantage of marker-assisted selection over field evaluations is that MAS can be done at any time of year regardless of the growing season. Moreover, environmental effects are irrelevant to marker-assisted selection.

A plant breeder can advantageously use molecular markers to identify individuals containing any of the targeted genome edits by identifying marker alleles that show a statistically significant probability of co-segregation with male sterility, manifested as linkage disequilibrium. This is referred to as marker assisted selection (MAS). Thus, methods for the selection of mutant wheat plants that are homozygous or heterozygous for a mutation in the Ms9, Ms 22, Ms 26, Ms 45 gene, are also provided.

To perform MAS, a nucleic acid corresponding to the marker nucleic acid allele is detected in a biological sample from a plant to be selected. This detection can take the form of hybridization of a probe nucleic acid to a marker allele or amplicon thereof, e.g., using allele-specific hybridization, Southern analysis, northern analysis, in situ hybridization, hybridization of primers followed by PCR amplification of a region of the marker, DNA sequencing of a PCR amplification product, or the like. For any of the marker sequences described herein, one of ordinary skill in the art would understand how to obtain the allele at a marker locus in a particular wheat line or variety using known DNA amplification and sequencing techniques. For the purposes described herein, the lines or varieties that were used were publicly available. Hence, DNA could be obtained, and one of ordinary skill in the art could either use the provided primers or develop primers from the provided reference sequence to amplify and obtain the sequence at each marker locus from each line or variety.

After the presence (or absence) of a particular marker allele in the biological sample is verified, the plant is selected and is crossed to a second plant, optionally a wheat plant from an elite line. The progeny plants produced by the cross can be evaluated for that specific marker allele, and only those progeny plants that have the desired marker allele will be chosen.

Through marker assisted selection, a plant breeder can follow the presence of the male sterility trait through controlled crosses to obtain, when desired, a new plant containing a Ms9, Ms 22, Ms 26, Ms 45 gene mutation in either the homozygous or heterozygous state, thus maintaining the Ms9, Ms 22, Ms 26, Ms 45 gene mutations. In addition, marker assisted selection can be used to produce mutant male sterile seed parents that would be used as female, i.e. plants that need pollination by a pollen donor plant, to produce seeds of commercial interest. Alternatively, marker assisted selection could be used to produce F1 hybrids containing a Ms9, Ms 22, Ms 26, Ms 45 gene mutation in the heterozygous state.

Any of the markers provided herein, as well as any marker linked to and associated with any of those markers, can be used for marker assisted selection of the male sterility trait.

Compositions and methods for restoring male fertility to a male-sterile plant are provided. In some examples, the male-sterile plants are homozygous recessive for the fertility gene of Ms9, Ms22, Ms26 or Ms45. In some embodiments, the male-sterile phenotype is caused by the introduction of genetic modification of a target site located in a male fertility gene locus of Ms9, Ms22, Ms26 or Ms45 in a plant cell's genome. In some examples, the wheat genomes (A, B, and D) contain homologous genes that have similar gene structure and function, requiring triple mutants to result in a male-sterile phenotype. Male-sterile plants may be created using the methods and compositions described herein and those known to one skilled in the art. In some embodiments, provided herein are compositions and methods to complement and restore male fertility to wheat plants containing mutations or introduced genetic modifications in MS9, MS22, MS26 or MS45 genes or MS9, MS22, MS26 or MS45 locus.

Male-sterile plants may be restored to male fertility when a functional copy of the Ms9, Ms22, Ms26 or Ms45 fertility gene, from the same or different species, is used to complement the Ms9, Ms22, Ms26 or Ms45 mutation or introduced genetic modification. See, for example, Example 3 which demonstrates that a single copy of a rice ms45 gene can restore fertility and maintain tams45-abd triple homozygous wheat mutants.

When the male-fertility MS9, MS22, MS26 or MS45 polynucleotide, fragment or variant is expressed, the plant is able to successfully produce mature pollen grains because the male-fertility polynucleotide restores the plant to a fertile condition. In some examples, the MS9, MS22, MS26 or MS45 polynucleotide, fragment, or variant thereof is maintained in a hemizygous state in a plant, so that only certain daughter cells will inherit the MS9, MS22, MS26 or MS45 polynucleotide, fragment, or variant in the process of pollen grain formation. Hemizygosity is a genetic condition existing when there is only one copy of a gene (or set of genes) with no allelic counterpart.

In some embodiments, the male-fertility MS9, MS22, MS26 or MS45 polynucleotide, fragment, or variants thereof, is operably linked to a promoter, to express the MS9, MS22, MS26 or MS45 polynucleotide, fragment, or variant and modulate, e.g., restore, the male fertility of a plant. In some examples, the MS9, MS22, MS26 or MS45 polynucleotide, fragment, or variant are expressed from an expression cassette. In some embodiments, the male-fertility MS9, MS22, MS26 or MS45 polynucleotides or expression cassette disclosed herein are maintained in a hemizygous state in a plant.

In particular embodiments, the male-fertility MS9, MS22, MS26 or MS45 polynucleotide, or fragment or variant thereof, is operably linked to a promoter. In certain embodiments, plant promoters can preferentially initiate transcription in certain tissues, such as stamen, anther, filament, and pollen, or developmental growth stages, such as sporogenous tissue, microspores, and microgametophyte. Such plant promoters are referred to as “tissue-preferred,” “cell-type-preferred,” or “growth-stage preferred.” Promoters which initiate transcription only in certain tissue are referred to as “tissue-specific.” Likewise, promoters which initiate transcription only at certain growth stages are referred to as “growth-stage-specific.” A “cell-type-specific” promoter drives expression only in certain cell types in one or more organs, for example, stamen cells, or individual cell types within the stamen such as anther, filament, or pollen cells.

A “male-fertility promoter” may initiate transcription exclusively or preferentially in a cell or tissue involved in the process of microsporogenesis or microgametogenesis. Male-fertility polynucleotides disclosed herein, and active fragments and variants thereof, can be operably linked to male-tissue-specific or male-tissue-preferred promoters including, for example, stamen-specific or stamen-preferred promoters, anther-specific or anther-preferred promoters, pollen-specific or pollen-preferred promoters, tapetum-specific promoters or tapetum-preferred promoters, and the like. Promoters can be selected based on the desired outcome. For example, the Ms9, Ms22, Ms26, or Ms45 polynucleotides can be operably linked to constitutive, tissue-preferred, growth stage-preferred, or other promoters for expression in plants.

In one embodiment, the promoters may be those which express an operably-linked Ms9, Ms22, Ms26, or Ms45 polynucleotide exclusively or preferentially in the male tissues of the plant. Any suitable male-fertility tissue-preferred or tissue-specific promoter may be used in the process; and any of the many such promoters known to one skilled in the art may be employed. One such promoter is the 5126 promoter, which preferentially directs expression of the polynucleotide to which it is linked to male tissue of the plants, as described in U.S. Pat. Nos. 5,837,851 and 5,689,051. Other exemplary promoters include the native promoter of Ms9, Ms22, Ms26, or Ms45, including those known and disclosed herein in SEQ ID NO:2, 6, 10, 14, 18, 22, 26, 30, 34, 38, 42, 46, 50, 54, 58, 62, 66, 70, 74, and 78.

In some examples, a termination region is operably linked to the male-fertility MS9, MS22, MS26 or MS45 polynucleotide, fragment or variant. In some examples, the terminator region is the native terminator of Ms9, Ms22, Ms26, or Ms45, including those known and disclosed herein.

Where appropriate, the MS9, MS22, MS26 or MS45 polynucleotides may be optimized for increased expression in the plant. That is, the MS9, MS22, MS26 or MS45 polynucleotides can be synthesized or altered to use plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

A male-fertility MS9, MS22, MS26 or MS45 polynucleotide disclosed herein can be provided in an expression cassette for expression in a plant of interest. The cassette can include 5′ and 3′ regulatory sequences operably linked to a male-fertility polynucleotide as disclosed herein. In some examples, the expression cassette includes in addition to the polynucleotide encoding the Ms9, Ms22, Ms26, or Ms45 polypeptide a male-gamete-disruptive polynucleotide, that is, a polynucleotide which interferes with the function, formation, or dispersal of male gametes. A male-gamete-disruptive polynucleotide can operate to prevent function, formation, or dispersal of male gametes by any of a variety of methods. By way of example but not limitation, this can include use of polynucleotides which encode a gene product such as DAM-methylase or barnase (See, for example, U.S. Pat. No. 5,792,853 or 5,689,049; PCT/EP89/00495); encode a gene product which interferes with the accumulation of starch, degrades starch, or affects osmotic balance in pollen, such as alpha-amylase (See, for example, U.S. Pat. Nos. 7,875,764; 8,013,218; 7,696,405, 8,614,367); inhibit formation of a gene product important to male gamete function, formation, or dispersal (See, for example, U.S. Pat. Nos. 5,859,341; 6,297,426). In some examples, the male-gamete-disruptive polynucleotide is operably linked to a male-tissue-preferred promoter.

When the expression cassette is introduced into the plant in a hemizygous condition, only certain daughter cells will inherit the expression cassette in the process of pollen grain formation. The daughter cells that inherit the expression cassette containing the male-fertility MS9, MS22, MS26 or MS45 polynucleotide will not develop into mature pollen grains due to the male-tissue-preferred expression of the stacked encoded male-gamete-disruptive gene product. Those pollen grains that do not inherit the expression cassette will continue to develop into mature pollen grains and be functional, but will not contain the male-fertility polynucleotide of the expression cassette and therefore will not transmit the male-fertility polynucleotide to progeny through pollen. See, for example, U.S. Pat. Nos. 7,875,764; 8,013,218; 7,696,405, 8,614,367, herein incorporated by reference in its entirety.

In one embodiment, the homozygous recessive condition of a male-sterile plant produced using methods described herein is maintained. A method of maintaining the homozygous recessive condition of a male-sterile plant may include fertilizing the homozygous recessive male-sterile plant with pollen from a plant expressing (1) a MS9, MS22, MS26, or MS45 fertility gene that when the gene is expressed in the plant restores male fertility to the male-sterile plant and (2) a polynucleotide sequence that inhibits the function or formation of viable male gametes, which are driven by promoters that preferentially expresses the sequence in male plant cells, such as male gametes. See, for example, U.S. Pat. No. 8,614,367. The progeny produced will continue to be male sterile as a result of maintaining homozygosity for the fertility gene, e.g. MS9, MS22, MS26, and MS45. The progeny will not contain the introduced restoring fertility gene-male gamete inhibition construct. The plant having the restorer nucleotide sequence may be self-fertilized, that is pollen from the plant transferred to the flower of the same plant to achieve the propagation of the restorer plants. Note that in referring to “self fertilization”, it includes the situation where the plant producing the pollen is fertilized with that same pollen, and the situation where two or more identical inbred plants are planted together and pollen from the identical inbred plant pollinate a different identical inbred plant. The pollen will not have the restoring transgene construct but it will be contained in 50% of the ovules (the female gamete). The seed resulting from the self-fertilization can be planted, and selection made for the seed having the restoring fertility gene-male gamete inhibition construct. Selection will allow for the identification of those plants produced from the seed having the restoring fertility gene-male gamete inhibition construct.

Also provided herein are wheat U6 promoters. As used herein, the terms “wheat U6 promoter”, “wheat U6 RNA polymerase III promoter”, and “wheat U6 snRNA polymerase III promoter” are used interchangeably herein. The term “wheat U6 promoter” includes a native wheat promoter (or a functional fragment thereof) and an engineered sequence comprising at least a fragment of the native wheat promoter with a DNA linker attached to facilitate cloning. A DNA linker may comprise a restriction enzyme site.

In some aspects, the wheat U6 promoter may be used to express a coding sequence or functional RNA. Functional RNA includes, but is not limited to, crRNA, tracerRNA, guide RNA, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene or functional RNA in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation of the promoter may also have promoter activity.

“Promoter functional in a plant” is a promoter capable of controlling transcription in plant cells whether or not its origin is from a plant cell.

“Constitutive promoter” refers to promoters active in all or most tissues or cell types of a plant at all or most developing stages. As with other promoters classified as “constitutive” (e.g., ubiquitin), some variation in absolute levels of expression can exist among different tissues or stages.

The wheat U6 promoter nucleotide sequences and methods disclosed herein are useful in regulating constitutive expression of any heterologous nucleotide sequences (such as but not limited to RNA sequences) in a host plant in order to alter the phenotype of a plant. In one embodiment, this disclosure concerns a recombinant DNA construct comprising a nucleotide sequence comprising any of the sequences set forth in SEQ ID NOs: 451, 452, or 453, or a functional fragment thereof, operably linked to at least one heterologous sequence, wherein said nucleotide sequence is a constitutive promoter.

While this nucleotide sequence is heterologous to the promoter sequence, it may be homologous, or native, or heterologous, or foreign, to the plant host. However, it is recognized that the instant wheat U6 promoters may be used with their native coding sequences to increase or decrease expression resulting in a change in phenotype in a transformed seed. The terms “heterologous nucleotide sequence”, “heterologous polynucleotide sequence”, “heterologous sequence”, “heterologous nucleic acid fragment”, and “heterologous nucleic acid sequence” are used interchangeably herein.

The present disclosure encompasses recombinant DNA constructs comprising functional fragments of the wheat U6 promoter sequences or Ms9, MS22, MS26, or MS45 promoter sequences disclosed herein.

With regard to promoters, the term a “functional fragment” refer to a portion or subsequence of the promoter sequence of the present disclosure in which the ability to initiate transcription or drive gene expression (such as to produce a certain phenotype) of a polynucleotide. For example, a “functional fragment” of a wheat U6 promoter would retain the ability to express a functional RNA.

Fragments can be obtained via methods such as site-directed mutagenesis and synthetic construction. As with the provided promoter sequences described herein, the functional fragments operate to promote the expression of an operably linked heterologous polynucleotide sequence, forming a recombinant DNA construct (also, a chimeric gene). For example, the wheat U6 promoter fragment can be used in the design of recombinant DNA constructs to produce guide RNAs capable of interacting with Cas enducleases and forming a guideRNA/Cas endonuclease complex. Recombinant DNA constructs can be designed for use in co-suppression or antisense by linking a promoter fragment in the appropriate orientation relative to a heterologous polynucleotide sequence.

In an embodiment of the present disclosure, the promoters disclosed herein can be modified. Those skilled in the art can create promoters that have variations in the polynucleotide sequence. The polynucleotide sequence of the promoters of SEQ ID NO: 2, 6, 10, 14, 18, 22, 26, 30, 34, 38, 42, 46, 50, 54, 58, 62, 66, 70, 74, 78, 451, 452, or 453 may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

A “variant promoter”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

The promoter sequences disclosed herein may be comprised in a recombinant DNA construct.

Moreover, the skilled artisan recognizes that substantially similar nucleic acid sequences encompassed by this disclosure are also defined by their ability to hybridize, under moderately stringent conditions (for example, 0.5×SSC, 0.1% SDS, 60 degree C.) with the sequences exemplified herein, or to any portion of the nucleotide sequences reported herein and which are functionally equivalent to the promoter of the disclosure. Estimates of such homology are provided by either DNA-DNA or DNA-RNA hybridization under conditions of stringency as is well understood by those skilled in the art (Hames and Higgins, Eds.; In Nucleic Acid Hybridization; IRL Press: Oxford, U K, 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6.times.SSC, 0.5% SDS at room temperature for 15 min, then repeated with 2×SSC, 0.5% SDS at 45 degree C. for 30 min, and then repeated twice with 0.2×SSC, 0.5% SDS at 50 degree C. for 30 min. Another set of stringent conditions uses higher temperatures in which the washes are identical to those above except for the temperature of the final two 30 min washes in 0.2×SSC, 0.5% SDS was increased to 60 degree C. Another set of highly stringent conditions uses two final washes in 0.1×SSC, 0.1% SDS at 65 degree C.

Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 71% to 100%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%. Also included herein are promoter that may comprise a polynucleotide having at least 85%, 90%, 95%, 96%, 97%, 98%, or 99% sequence identity to SEQ ID NO: 2, 6, 10, 14, 18, 22, 26, 30, 34, 38, 42, 46, 50, 54, 58, 62, 66, 70, 74, 78, 451, 452, or 453. In some aspects, the % sequence identity is determined over the entire length of the nucleotide sequence, for example, over the full-length.

A “substantially homologous sequence” refers to variants of the disclosed sequences such as those that result from site-directed mutagenesis, as well as synthetically derived sequences. A substantially homologous sequence of the present disclosure also refers to those fragments of a particular promoter nucleotide sequence disclosed herein that operate to promote the expression of an operably linked heterologous nucleic acid fragment. These promoter fragments may comprise at least about 100 contiguous nucleotides, 150 contiguous nucleotides, 200 contiguous nucleotides, 250 contiguous nucleotides, 300 contiguous nucleotides, 350 contiguous nucleotides, 400 contiguous nucleotides, 450 contiguous nucleotides, 500 contiguous nucleotides, 550 contiguous nucleotides, 600 contiguous nucleotides, 650 contiguous nucleotides, 700 contiguous nucleotides, 750 contiguous nucleotides, 800 contiguous nucleotides, 850 contiguous nucleotides, 900 contiguous nucleotides, 950 contiguous nucleotides, or 1000 contiguous nucleotides of the particular promoter nucleotide sequence disclosed herein, for example, of SEQ ID NO: 2, 6, 10, 14, 18, 22, 26, 30, 34, 38, 42, 46, 50, 54, 58, 62, 66, 70, 74, 78, 451, 452, or 453. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis, et al., (1987) Methods Enzymol. 155:335-350, and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present disclosure.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED-4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Also included herein is a recombinant DNA construct comprising a MS9, MS22, MS26, or MS45 or wheat U6 promoter. This disclosure also concerns a recombinant DNA construct comprising a promoter wherein said promoter consists essentially of the nucleotide sequence set forth in SEQ ID NOS: 2, 6, 10, 14, 18, 22, 26, 30, 34, 38, 42, 46, 50, 54, 58, 62, 66, 70, 74, 78, 451, 452, or 453 or a functional fragment of SEQ ID NOS: 2, 6, 10, 14, 18, 22, 26, 30, 34, 38, 42, 46, 50, 54, 58, 62, 66, 70, 74, 78, 451, 452, or 453. This disclosure also concerns an isolated polynucleotide comprising a promoter wherein said promoter comprises the nucleotide sequence set forth in SEQ ID NOS: 2, 6, 10, 14, 18, 22, 26, 30, 34, 38, 42, 46, 50, 54, 58, 62, 66, 70, 74, 78, 451, 452, or 453, or a functional fragment of SEQ ID NOS: 2, 6, 10, 14, 18, 22, 26, 30, 34, 38, 42, 46, 50, 54, 58, 62, 66, 70, 74, 78, 451, 452, or 453.

In some aspects, one of the wheat U6 promoters disclosed herein is operably linked to a nucleotide sequence encoding a suitable functional RNA such as but not limited to a crRNA, a tracerRNA or a single guide RNA. This expression cassette may be transformed into an appropriate plant for expression of the promoter. There are a variety of appropriate plants which can be used as a host for transformation that are well known to those skilled in the art, including the dicots, Arabidopsis, tobacco, soybean, oilseed rape, peanut, sunflower, safflower, cotton, tomato, potato, cocoa and the monocots, corn, wheat, rice, barley and palm. The cassette may be tested for expression of the wheat U6 promoter in various cell types of transgenic plant tissues, e.g., leaves, roots, flowers, seeds, transformed with the chimeric U6 promoter-reporter gene expression cassette by assaying for expression of the reporter gene product.

In another embodiment, this disclosure concerns host cells comprising either the recombinant DNA constructs of the disclosure as described herein or isolated polynucleotides of the disclosure as described herein. Examples of host cells which can be used to practice the disclosure include, but are not limited to, yeast, bacteria, and plants. Plasmid vectors comprising the instant recombinant DNA construct can be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene.

Non-limiting examples of methods and compositions disclosed herein are as follows:
1. A recombinant DNA construct comprising a nucleotide sequence comprising any one of the sequence set forth in SEQ ID NO: 451, 452, or 453, or a functional fragment thereof, operably linked to at least one heterologous sequence, wherein said nucleotide sequence is a promoter.
2. The recombinant DNA construct of embodiment 1, wherein said nucleotide sequence has at least 95% identity, based on the Clustal V method of alignment with pairwise alignment default parameters (KTUPLE=2, GAP PENALTY=5, WINDOW=4 and DIAGONALS SAVED=4), when compared to any one of the sequence set forth in SEQ ID NO: 451, 452, or 453.
3. A vector comprising the recombinant DNA construct of embodiment 1.
4. A cell comprising the recombinant DNA construct of embodiment 1.
5. The cell of embodiment 4, wherein the cell is a plant cell.
6. A transgenic plant having stably incorporated into its genome the recombinant DNA construct of embodiment 1.
7. The transgenic plant of embodiment 6 wherein said plant is a monocot plant.
8. The transgenic plant of embodiment 7 wherein the plant is wheat.
9. A transgenic seed produced by the transgenic plant of embodiment 7, wherein the transgenic seed comprises the recombinant DNA construct.
10. The recombinant DNA construct of embodiment 1 wherein the at least one heterologous sequence codes for a functional RNA molecule selected from the group consisting of crRNA, tracrRNA and guide-RNA.
11. The recombinant DNA construct of embodiment 1, wherein the at least one heterologous sequence encodes a single guide RNA that is capable of forming a guide RNA/Cas endonuclease complex, wherein said guide-RNA hybridizes to a DNA target site.
12. A method of expressing a functional RNA in a plant comprising:
a) introducing the recombinant DNA construct of embodiment 1 into the plant, wherein the at least one heterologous sequence encodes a functional RNA;
b) growing the plant of step a); and
c) selecting a plant displaying expression of the functional RNA of the recombinant DNA construct.
13. The method of Embodiment 12 wherein the plant is a monocot plant.
14. The method of Embodiment 12 wherein the plant is a wheat plant.
15. A plant stably transformed with a recombinant DNA construct comprising a wheat U6 polymerase III promoter and a heterologous nucleic acid fragment operably linked to said U6 polymerase Ill promoter, wherein said U6 polymerase III promoter is a capable of controlling expression of said heterologous nucleic acid fragment in a plant cell, and further wherein said U6 polymerase III promoter comprises any of the sequences set forth in SEQ ID NO: 451, 452, or 453.
16. A recombinant DNA construct comprising a wheat U6 polymerase III promoter driving a heterologous nucleic acid fragment encoding a guide polynucleotide, a crRNA, or a tracrRNA, wherein said promoter comprises any of the sequences set forth in SEQ ID NO: 451, 452, or 453, or a functional fragment thereof.
17. A method for modifying a target site in the genome of a plant cell, the method comprising introducing a guide RNA and a Cas endonuclease into said plant cell, wherein said guide RNA and Cas endonuclease are capable of forming a complex that enables the Cas endonuclease to introduce a double strand break at said target site, wherein said guide RNA is expressed by a recombinant DNA construct comprising a promoter comprising any of the sequences set forth in SEQ ID NO451, 452, or 453, or a functional fragment thereof.
18. A recombinant DNA construct comprising a wheat U6 polymerase III promoter driving a heterologous nucleic acid fragment encoding a guide polynucleotide comprising: (i) a first nucleotide sequence domain that is complementary to a nucleotide sequence in a target DNA; and, (ii) a second nucleotide sequence domain that interacts with a Cas endonuclease, wherein the first nucleotide sequence domain and the second nucleotide sequence domain are composed of deoxyribonucleic acids (DNA), ribonucleic acids (RNA), or a combination thereof, wherein the guide polynucleotide does not solely comprises ribonucleic acids.
1. A recombinant DNA construct comprising a nucleotide sequence comprising any one of the sequence set forth in SEQ ID NO: 2, 6, 10, 14, 18, 22, 26, 30, 34, 38, 42, 46, 50, 54, 58, 62, 66, 70, 74, or 78, or a functional fragment thereof, operably linked to at least one heterologous sequence, wherein said nucleotide sequence is a promoter.
2. The recombinant DNA construct of embodiment 1, wherein said nucleotide sequence has at least 95% identity, based on the Clustal V method of alignment with pairwise alignment default parameters (KTUPLE=2, GAP PENALTY=5, WINDOW=4 and DIAGONALS SAVED-4), when compared to any one of the sequence set forth in SEQ ID NO: 2, 6, 10, 14, 18, 22, 26, 30, 34, 38, 42, 46, 50, 54, 58, 62, 66, 70, 74, or 78.
3. A vector comprising the recombinant DNA construct of embodiment 1.
4. A cell comprising the recombinant DNA construct of embodiment 1.
5. The cell of embodiment 4, wherein the cell is a plant cell.
6. A transgenic plant having stably incorporated into its genome the recombinant DNA construct of embodiment 1.
7. The transgenic plant of embodiment 6 wherein said plant is a monocot plant.
8. The transgenic plant of embodiment 7 wherein the plant is wheat.
9. A transgenic seed produced by the transgenic plant of embodiment 7, wherein the transgenic seed comprises the recombinant DNA construct.
10. The recombinant vector where the promoter is a male-tissue preferred or male-tissue specific promoter.

### Definitions

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Cosuppression” generally refers to the production of sense RNA transcripts capable of suppressing the expression of the target gene or gene product. “Sense” RNA generally refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

The terms “full complement” and “full-length complement” are used interchangeably herein, and refer to a complement of a given nucleotide sequence, wherein the complement and the nucleotide sequence consist of the same number of nucleotides and are 100% complementary.

“Gamete” refers to a reproductive cell having the 1n set (haploid number) of chromosomes that can fuse with another gamete of the opposite sex during fertilization in organisms undergoing sexual reproduction. As used herein, a gamete in organisms undergoing asexual reproduction refers to a cell having a 2n number (an unreduced number) of chromosomes.

The term “gene” as used herein refers to a polynucleotide that is expressed by at least one of transcription and translation. An example of a gene is a nucleic acid fragment capable of being transcribed into mRNA or translated into a protein. A “gene” may or may not include a coding region or a regulatory sequence of a 5′-non coding sequence and a 3′-non coding sequence in addition to the coding region. For example, a Ms9 gene refers to a Ms9 polynucleotide that is expressed by at least one of transcription and translation.

As used herein, the term “gene locus” refers to the position of a gene on a genome. For example, Ms9 gene locus refers to the position of a Ms9 gene on genome.

The term “genome” refers to the entire complement of genetic material (genes and non-coding sequences) that is present in each cell of an organism, or virus or organelle; and/or a complete set of chromosomes inherited as a (haploid) unit from one parent.

“Heterologous” with respect to sequence means a sequence that originates from a foreign species, or, if from the same species, is substantially modified from its native form in composition and/or genomic locus by deliberate human intervention.

The term “introduced” in the context of inserting a nucleic acid into a cell,” and includes reference to the incorporation of a nucleic acid or nucleic acid fragment into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. The term also embraces recombinant polynucleotides and chemically synthesized polynucleotides.

As used herein, a “male sterile plant” is a plant that does not produce male gametes that are viable or otherwise capable of fertilization.

The term “miRNA* sequence” refers to a sequence in the miRNA precursor that is highly complementary to the miRNA sequence. The miRNA and miRNA* sequences form part of the stem region of the miRNA precursor hairpin structure.

The terms “monocot” and “monocotyledonous plant” are used interchangeably herein. A monocot of the current disclosure includes the Gramineae.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. In certain embodiments, sequence identity may be based on the Clustal V or Clustal W method of alignment. The term “about” when used herein in context with percent sequence identity means+/−1.0%.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollen, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

“Polypeptide”, “peptide”, “amino acid sequence” and “protein” are used interchangeably herein to refer to a polymer of amino acid residues.

“Progeny” comprises any subsequent generation of a plant.

“Promoter functional in a plant” is a promoter capable of controlling transcription in plant cells whether or not its origin is from a plant cell.

As used herein “promoter” includes reference to a region of DNA upstream from the start of transcription and involved in recognition and binding of RNA polymerase and other proteins to initiate transcription.

A “plant promoter” is a promoter capable of initiating transcription in plant cells.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” means conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence. The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50 ºC and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68 ºC, 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The terms “suppress”, “suppressed”, “suppression”, “suppressing” and “silencing”, are used interchangeably herein and include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches and the like.

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

The term “under stringent conditions” means that two sequences hybridize under moderately or highly stringent conditions.

As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some embodiments, the wheat plant is suitable for commercial production of grain, such as commercial varieties of hexaploid wheat or durum wheat, having suitable agronomic characteristics which are known to those skilled in the art.

The meaning of abbreviations is as follows: “see” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “umole” mean micromole(s), “g” means gram(s), “μg” means microgram(s), “ng” means nanogram(s), “U” means unit(s), “bp” means base pair(s) and “kb” means kilobase(s).

Also, as described herein, for each example or embodiment that cites a guide RNA, a similar guide polynucleotide can be designed wherein the guide polynucleotide does not solely comprise ribonucleic acids but wherein the guide polynucleotide comprises a combination of RNA-DNA molecules or solely comprises DNA molecules.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1: Maize Optimized Expression Cassettes for Guide RNA/Cas Endonuclease Based Genome Modification in Wheat Plants

For genome engineering applications, the type II CRISPR/Cas system minimally requires the Cas9 protein and a duplexed crRNA/tracrRNA molecule or a synthetically fused crRNA and tracrRNA (guide RNA) molecule for DNA target site recognition and cleavage (See, published PCT application, WO2015026883 published on Feb. 26, 201). Described herein is a guideRNA/Cas endonuclease system that is based on the type II CRISPR/Cas system and consists of a Cas endonuclease and a guide RNA (or duplexed crRNA and tracrRNA) that together can form a complex that recognizes a genomic target site in a wheat plant and introduces a double-strand-break into said target site.

To test the guide RNA/Cas endonuclease system in wheat, the CRISPR/Cas system for maize as described by Cigan et al. was used (See, published PCT application, WO2015026883 published on Feb. 26, 2015). Briefly, this system consists of a maize codon optimized Cas9 gene from Streptococcus pyogenes M1 GAS (SF370) and the potato ST-LS1 intron was introduced in order to eliminate its expression in E. coli and Agrobacterium. To facilitate nuclear localization of the Cas9 protein in cells, monopartite amino terminal nuclear localization signal from Simian virus 40 (SV40), and bipartite VirD2 T-DNA border endonuclease carboxyl terminal nuclear localization signal from Agrobacterium tumefaciens were incorporated at the amino and carboxyl-termini of the Cas9 open reading frame, respectively. The maize optimized Cas9 gene was operably linked to a constitutive promoter from maize by standard molecular biological techniques.

The second component necessary to form a functional guide RNA/Cas endonuclease system for genome engineering applications is a duplex of the crRNA and tracrRNA molecules or a synthetic fusing of the crRNA and tracrRNA molecules, a guide RNA. To confer efficient guide RNA expression (or expression of the duplexed crRNA and tracrRNA), the maize U6 polymerase III promoter and maize U6 polymerase III terminator were operably fused to the termini of a guide RNA using standard molecular biology techniques.

Two crRNAs, a 20 nucleotide molecule (GGTCATGCAGCGCTGGCCGA SEQ ID: 222) and a 21 nucleotide molecule (GCAGCGCTGGCCGAGGGACAA SEQ ID:223), containing a region complementary to one strand of the double strand DNA target (referred to as the variable targeting domain) was designed upstream of a PAM sequence (GGG) and (CGG) respectively. Guide RNA (gRNA) also consisted of a 77 nucleotide tracrRNA fusion transcripts used to direct Cas9 to cleave sequence of interest. The construct also included a DsRed2 gene under control of the maize Ubiquitin promoter (see, e.g., U.S. Pat. No. 5,525,716) and PINII terminator for selection during transformation. This construct was transformed directly into wheat by Agrobacterium-mediated transformation methods as described in Cigan A M et al. (2017), yielding several independent T-DNA insertion events for construct evaluation. See Cigan et al, Targeted mutagenesis of a conserved anther-expressed P450 gene confers male sterility in monocots. Plant Biotechology Journal In Press: (2017) 15:379-389.

T0 wheat plants containing variable number of transgenes were grown to maturity and seed harvested. T1 plants were grown and examined for the presence of NHEJ mutations by deep sequencing. A total of 28 T1 plants were identified with 35 different mutations, including three plants with mutations in two different genomes and two plants with a mutation in each of three genomes (Table 3).

TABLE 3
CRISPR-CAS induced NHEJ mutations in wheat Ms45 gene

| | | | | | | Cas9 |
|---|---|---|---|---|---|---|
| Plant | | | A- | B- | D- | TDNA |
| ID | Genotype | CRISPR | genome | genome | genome | Copy # |
| 1 | Fielder | MS45-CR1 | | | 20bp | 3 |
| | | | | | del | |
| 2 | Fielder | MS45-CR1 | | G ins | | 5 |
| 3 | Fielder | MS45-CR2 | 23 bp | | | 4 |
| | | | del | | | |
| 4 | Fielder | MS45-CR1 | | 27 bp | | 1 |
| | | | | indel | | |
| 5 | Fielder | MS45-CR1 | | | C ins | 3 |
| 6 | Fielder | MS45-CR1 | | | T ins | >5 |
| 7 | Fielder | MS45-CR1 | T ins | | | 2 |
| 8 | Fielder | MS45-CR1 | 18 bp | | | 5 |
| | | | indel | | | |
| 9 | Fielder | MS45-CR1 | 20 bp | A ins | | 2 |
| | | | del | | | |
| 10 | Fielder | MS45-CR2 | 18 bp | | | 7 |
| | | | del | | | |
| 11 | Fielder | MS45-CR1 | 7 bp | A ins | A ins | 5 |
| | | | del | | | |
| 12 | Fielder | MS45-CR1 | | | 20 bp | 2 |
| | | | | | del | |
| 13 | Fielder | MS45-CR1 | | | A ins | 1 |
| 14 | Fielder | MS45-CR1 | | | A ins | 2 |
| 15 | Fielder | MS45-CR1 | | | A ins | 8 |
| 16 | Fielder | MS45-CR2 | | | 10 bp | 2 |
| | | | | | ins/ | |
| | | | | | subst | |
| 17 | Fielder | MS45-CR2 | GA del | | | 1 |
| 18 | Fielder | MS45-CR1 | A ins* | | | 4 |
| | | | (A or | | | |
| | | | D) | | | |
| 19 | Fielder | MS45-CR1 | | | T ins | 2 |
| 20 | Fielder | MS45-CR1 | | G ins | | 4 |
| 21 | Fielder | MS45-CR2 | 23 bp | | | 2 |
| | | | del | | | |
| 22 | Fielder | MS45-CR2 | | 23 bp | | 1 |
| | | | | del | | |
| 23 | SBC0456D* | MS45-CR1 | T ins | | | 4 |
| | | | (A or | | | |
| | | | D) | | | |
| 24 | SBC0456D | MS45-CR1 | A ins | A ins | | 2 |
| | | | (A or | | | |
| | | | D) | | | |
| 25 | SBC0456D | MS45-CR1 | AA ins | A ins | T ins | 1 |
| | | | (A or | | (A or D) | |
| | | | D) | | | |
| 26 | SBC0456D | MS45-CR1 | | 20 bp | | 1 |
| | | | | del | | |
| 27 | SBC0456D | MS45-CR1 | T ins | | | 1 |
| | | | (A or | | | |
| | | | D) | | | |
| 28 | SBC0456D | MS45-CR1 | T ins | | G ins | 1 |
| | | | (A or | | (a or D) | |
| | | | D) | | | |

SBC0456D is a pioneer elite spring wheat variety, see US published patent application no. 20140173781.

### Example 2: Simultaneous Tams45 Mutations in A-, B- and D-Genomes with CRISPR-Cas9 Results in Male Sterile Wheat

This example shows that triple homozygous mutant plants of TaMs45 gene derived from CRISPR-Cas9 induced mutations in the A, B, and D-genomes result in male sterile phenotype.

The genetic nature of TaMs45 mutant alleles in hexaploid wheat is denoted as follows:

Homozygous wild-type TaMs45 alleles in the genome A, B, and D are designated as TaMs45-ABD.

Homozygous mutant alleles are designated by a small letter; for example A-genome homozygous mutations are designated as TaMs-aBD.

Heterozygous mutant alleles are designated by a double letter; for example A-genome heterozygous mutations are designated as TaMs-AaBD.

Triple homozygous mutant alleles are designated as Tams-abd.

Triple heterozygous mutant alleles are designated as TaMs-AaBbDd.

Two plants with heterozygous mutations in all three homeologs (TaMs-AaBbDd), plant #11 and plant #25 in Table 3, were identified and allowed to self-pollinate to produce T2-T4 generations. Progeny from these generations was screened by deep sequencing and 17 triple homozygous mutant plants were identified (Tams45-abd; Table 4). Four plants were identified in the T4 progeny of plant #25 which were homozygous for all three mutations but without Cas9-sgRNA T-DNA (Table 5).

Table 4. Mutations and deep sequencing read counts from the three TaMs45 homeologs of Plant #11 and Plant #25. Bold text represents insertions, (−) represents a deletion mutation or a gap in the WT sequence due to insertion mutations

Triple homozygous mutants (Tams45-abd) were similar to wild-type plants in vegetative growth and flowering characteristics. However, the anthers in Tams45-abd plants were shrunken in shape and did not extrude or dehisce like anthers from

TABLE 4
Mutations and deep sequencing read counts from the three TaMs45
homeologs of Plant #11 and Plant #25.

| | | Read | % of | Zygosity |
|---|---|---|---|---|
| Plant #11 | Mutation | Count | counts | in T1 |
| WT | TACAGGGAGGTCATGCAGCGCTGGCCGAGGGACAACGGCAGCCG | 172,654 | 47.4 | |
| | (SEQ ID NO: 265) | | | |
| A-genome | TACAGGGAGGTCATGCAG-------CGAGGGACAACGGCAGCCG | 59,842 | 16.4 | HET |
| | (SEQ ID NO: 266) | | | |
| B-genome | TACAGGGAGGTCATGCAGCGCTGGCACGAGGGACAACGGCAGCCG | 45,578 | 12.5 | HET |
| | (SEQ ID NO: 454) | | | |
| D-genome | TACAGGGAGGTCATGCAGCGCTGGCACGAGGGACAACGGCAGCCG | 73,592 | 20.2 | HET |
| | (SEQ ID NO: 455 | | | |
| Plant #25 | | | | |
| WT | TACAGGGAGGTCATGCAGCGCTGGC-CGAGGGACAACGGCAGCCG | 363,042 | 48.6 | |
| | (SEQ ID NO: 456) | | | |
| A-/D-genome | TACAGGGAGGTCATGCAGCGCTGGCAACGAGGGACAACGGCAGCCG | 134,946 | 18.1 | HET |
| | (SEQ ID NO: 457) | | | |
| B-genome | TACAGGGAGGTCATGCAGCGCTGGCACGAGGGACAACGGCAGCCG | 94,464 | 12.6 | HET |
| | (SEQ ID NO: 458) | | | |
| A-/D-genome | TACAGGGAGGTCATGCAGCGCTGGCTCGAGGGACAACGGCAGCCG | 124,340 | 16.6 | HET |
| | (SEQ ID NO: 459) | | | |

Bold text represents insertions, (-) represents a deletion mutation or a gap in the WT sequence due to insertion mutations

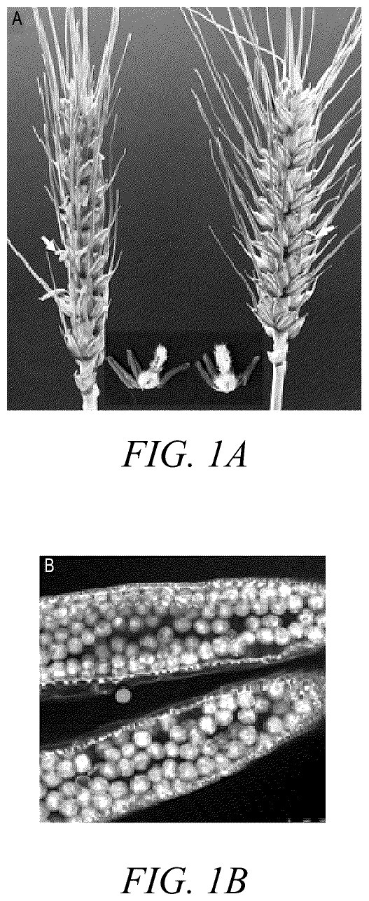

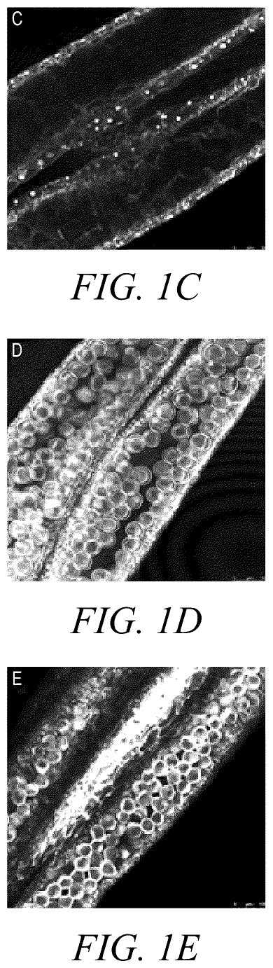

a fertile wild-type plant (See FIG. 1).

Seed from Tams45-abd individual plants was pooled and counted as a qualitative measure of male fertility. As shown in Table 5, at maturity plants containing triple homozygous ms45 mutations (tams45-abd), were male sterile did not set self-seed. (Note, seed observed in two of these plants was likely to due to open fertilization as these heads were not bagged prior to anthesis).

Previous studies of ms45 mutants in maize de<onstrated that the breakdown of microspores occurred shortly after tetrad release (Cigan et al., 2001). To determine whether plants containing mutations in the wheat Ms45 gene conferred a similar phenotype, anthers at the vacuolate stage of microspore development were examined for the presence of microspores using confocal microscopy. As shown in FIG. 1B and FIG. 1C, anthers from a wild-type plant contained many developing microspores, while anthers from a Tams45-abd plant contained only a few vestigial microspores.

Anther phenotype and microspore development observed in double homozygote-single heterozygote plants were normal and comparable to wild-type plants (FIG. 1D) and at maturity, the double homozygote-single heterozygotes showed seed set comparable to the wild-type plants (Table 5).

TABLE 5
Male fertility as indicated by seed set in TaMs45
mutants (T1, T2 and T3 generations).

| | Seed Set-Fertility | | |
|---|---|---|---|
| Genotype of | | | Seed per |
| TaMs45 homeologs¹ | Plants | Total Seed | Plant |
| Tams45-abd (T2; Plant #11) | 2 | 2 | ~0 |
| Tams45-abd (T3; Plant #25) | 11 | 0 | 0 |
| Tams45-abd (T4; Plant #25; | 4 | 0 | 0 |
| No Cas9-sgRNA) | | | |
| Total Tams45-abd plants | 17 | 2 | ~0 |
| Tams45-Aabd or -aBbd or | 2 | 235 | 118 |
| -abDd (Plant #11) | | | |
| Tams45-Aabd or -aBbd or | 9 | 1249 | 139 |
| -abDd (Plant #25) | | | |
| TaMs45-ABD | 5 | 855 | 171 |

¹(plant generation, T0 plant ID, No TDNA indicates the absence of Cas9-gRNA TDNA insertion).

### Example 3: A Single Copy of Monocot Ms45 Gene can Restore Fertility and Maintain Tams45-Abd Triple Homozygous Mutants

To propagate the Tams45-abd male sterile plants, it would be advantageous to generate a maintainer line. The main component required for maintaining a male sterile plant is the ability of an introduced wild-type copy of the gene to complement the mutations and render the plant fertile. To accomplish this, the rice Ms45 gene under control of the maize Ms45 promoter was linked to a DsRed2 gene under control of the maize ubiquitin promoter and also carrying a PINII terminator sequence (OsMs45-DsRED). This construct was transformed directly into wheat by Agrobacterium-mediated transformation methods as referenced elsewhere herein, yielding several independent T-DNA insertion events for evaluation. Wheat plants containing single-copy OsMs45-DsRED cassette were used as male plants to pollinate Tams45-abd plants. Seeds were harvested, planted, and progeny screened by PCR to confirm the presence of OsMs45-DsRED and TaMS45-A, -B and -D mutations and allowed to self-pollinate.

F2 seed were planted and progeny were genotyped for the OsMs45 gene and TaMs45 mutant alleles. Triple homozygous mutant plants with and without the complementation OsMs45-DsRED T-DNA were identified and analyzed for male fertility through microscopy of anthers and determination of seed set on individual plants. In total, six triple homozygous Tams45-abd mutant plants were analyzed, four with one copy of OsMs45-DsRED T-DNA and two lacking the T-DNA (Table 6). Pollen development in the anthers of plants with OsMs45 was similar to wild-type plants (FIG. 1E). All four Tams45-abd plants that carried OsMs45 were male fertile with seed set comparable to wild-type plants while the two plants without the OsMs45 showed negligible seed-set (Table 6; Note, seed observed in two of these plants was likely to due to open fertilization as these heads were not bagged prior to anthesis). This data exhibits that the male sterility observed in Tams45-abd plants is due to mutations in the TaMs45 gene and a single, transformed copy of Ms45 gene can complement and restore fertility and maintain the triple homozygous mutants.

TABLE 6
Complementation of TaMs45 triple hornozygous mutants.

| | Seed Set-Fertility | | | | |
|---|---|---|---|---|---|
| Genotype of | | Total | Seed per | Male | Male |
| TaMs45 homeologs¹ | Plants | Seed | Plant | fertile | sterile |
| Tams45-abd/OsMs45 | 4 | 565 | 141.5 | 4 | 0 |
| Tams45-abd | 2 | 3 | 1.5 | 0 | 2 |
| TaMs45-ABD | 5 | 855 | 171 | 5 | 0 |

### Example 4: Wheat U6 RNA Polymerase Ill Promoters

Wheat U6 RNA polymerase III promoter sequences were identified for use in expressing guide RNA to direct Cas9 nuclease (or other endonuclease) to a designated genomic site, see, for example, SEQ ID NOs: 451-453 identified on Triticum aestivum Chromosome 2, Chromosome 4 and Chromosome 5 respectively. The first nucleotide of a 20 bp variable targeting domain may contain a G residue for use by a RNA polymerase Ill for transcription see, for example, the target site sequences shown in Table 2. The wheat U6 snRNA polymerase Ill promoters may be synthesized and/or then cloned with an appropriate DNA sequence that encodes a guide RNA into an appropriate vector to make, for example, a DNA construct. This U6 RNA polymerase III promoter may be used to express other Cas endonuclease components, including but not limited to a Cas9 endonuclease.

1.-23. (canceled)

24. A method for restoring male fertility, the method comprising:
a) introducing a genetic modification into at least one or more endogenous wheat MS9, MS22, MS26 or MS45 polynucleotide sequences in a wheat plant cell, wherein the genetic modification confers male sterility to a wheat plant obtained from the plant cell, wherein the genetic modification is introduced by an endonuclease guided by at least one guide RNA; and
b) introducing into and expressing in the wheat plant cell one or more polynucleotides that encode a MS9, MS22, MS26, or MS45 polypeptide that confers male fertility to the male-sterile wheat plant obtained from the wheat plant cell.

25. The method of claim 24, the method further comprising obtaining a MS9, MS22, MS26, or MS45 male-fertile wheat plant from the wheat plant cell.

26. The method of claim 24, wherein the at least one or more endogenous wheat MS9 polynucleotide sequences is selected from the group consisting of:
(a) a polynucleotide comprising the sequence set forth in SEQ ID NO:1-3, 5-7, or 9-11;
(b) a polynucleotide having at least 85%, 90% or 95% sequence identity to SEQ ID NO: 1-3, 5-7, or 9-11;
(c) a polynucleotide that encodes a polypeptide having at least 85%, 90% or 95% sequence identity to SEQ ID NO: 4, 8, or 12; and
(d) a polynucleotide that encodes a polypeptide of SEQ ID NO: 4, 8, or 12.

27. The method of claim 24, wherein the at least one or more endogenous wheat MS22 polynucleotide sequence is selected from the group consisting of:
(a) a polynucleotide comprising the sequence set forth in SEQ ID NO:21-23, 25-27, or 29-31;
(b) a polynucleotide having at least 85%, 90% or 95% sequence identity to SEQ ID NO: 21-23, 25-27, or 29-31;
(c) a polynucleotide that encodes a polypeptide having at least 85%, 90% or 95% sequence identity to SEQ ID NO:24, 28, or 32; and
(d) a polynucleotide that encodes a polypeptide of SEQ ID NO: 24, 28, or 32.

28. The method of claim 24, wherein the at least one or more endogenous wheat MS26 polynucleotide sequence is selected from the group consisting of:
(a) a polynucleotide comprising the sequence set forth in SEQ ID NO:41-43, 45-47, or 49-51;
(b) a polynucleotide having at least 85%, 90% or 95% sequence identity to SEQ ID NO: 41-43, 45-47, or 49-51;
(c) a polynucleotide that encodes a polypeptide having at least 85%, 90% or 95% sequence identity to SEQ ID NO:44, 48, or 52; and
(d) a polynucleotide that encodes a polypeptide of SEQ ID NO: 44, 48, or 52.

29. The method of claim 24, wherein the at least one or more endogenous wheat MS45 polynucleotide sequence is selected from the group consisting of:
(a) a polynucleotide comprising the sequence set forth in SEQ ID NO:61-63, 65-67, or 69-71;
(b) a polynucleotide having at least 85%, 90% or 95% sequence identity to SEQ ID NO: 61-63, 65-67, or 69-71;
(c) a polynucleotide that encodes a polypeptide having at least 85%, 90% or 95% sequence identity to SEQ ID NO:64, 68, or 72; and
(d) a polynucleotide that encodes a polypeptide of SEQ ID NO: 64, 68, or 72.

30. The method of claim 24, wherein said the genetic modification is introduced by a TALEN, a meganuclease, a zinc finger nuclease, or a CRISPR-associated nuclease.

31. The method of claim 24, wherein the MS9, MS22, MS26, or MS45 polypeptide that confers male fertility to the male-sterile wheat plant is from a monocot.

32. The method of claim 24, wherein the MS9, MS22, MS26, or MS45 polypeptide that confers male fertility to the male-sterile wheat plant is from wheat or a wheat species.

33. The method of claim 24, wherein the MS9, MS22, MS26, or MS45 polypeptide that confers male fertility to the male-sterile wheat plant is from a species different than wheat.

34. The method of claim 24, wherein the genetic modification is introduced by a Cas9 endonuclease guided by at least one guide RNA.

35. The method of claim 24, wherein the genetic modification introduces one or more nucleotide substitutions, additions and/or deletions into at least one or more endogenous wheat MS9, MS22, MS26 or MS45 polynucleotide sequences.

36. The method of claim 24, further comprising crossing the male-fertile wheat plant with itself to produce wheat seed.

37. A wheat plant having restored male-fertility, the wheat plant comprising at least one altered target site that confers modified male-fertility to the wheat plant, wherein the at least one altered target site originated from a corresponding target site that was recognized and cleaved by a guideRNA/CRISPR-associated nuclease system, and wherein the at least one altered target site is located in or near one or more endogenous wheat male fertility polynucleotides of MS9, MS22, MS26, or MS45 and the altered target site affects the expression level of the one or more endogenous MS9, MS22, MS26, or MS45 polynucleotides so that the wheat plant is male-sterile and wherein the wheat plant comprises one or more polynucleotides that encodes a MS9, MS22, MS26, or MS45 polypeptide that confers male fertility to the male-sterile wheat plant when expressed in the wheat plant's male-tissue.

38. The wheat plant of claim 37, wherein the one or more endogenous wheat MS9 polynucleotide sequences is selected from the group consisting of:
(a) a polynucleotide comprising the sequence set forth in SEQ ID NO: 1-3, 5-7, or 9-11:
(b) a polynucleotide having at least 85%, 90% or 95% sequence identity to SEQ ID NO: 1-3, 5-7, or 9-11;
(c) a polynucleotide that encodes a polypeptide having at least 85%, 90% or 95% sequence identity to SEQ ID NO: 4, 8, or 12; and
(d) a polynucleotide that encodes a polypeptide of SEQ ID NO: 4, 8, or 12.

39. The wheat plant of claim 37, wherein the one or more endogenous wheat MS22 polynucleotide sequences is selected from the group consisting of:
(a) a polynucleotide comprising the sequence set forth in SEQ ID NO:21-23, 25-27, or 29-31;
(b) a polynucleotide having at least 85%, 90% or 95% sequence identity to SEQ ID NO: 21-23, 25-27, or 29-31;
(c) a polynucleotide that encodes a polypeptide having at least 85%, 90% or 95% sequence identity to SEQ ID NO:24, 28, or 32; and
(d) a polynucleotide that encodes a polypeptide of SEQ ID NO: 24, 28, or 32.

40. The wheat plant of claim 37, wherein the one or more endogenous wheat MS26 polynucleotide sequences is selected from the group consisting of:
(a) a polynucleotide comprising the sequence set forth in SEQ ID NO:41-43, 45-47, or 49-51;
(b) a polynucleotide having at least 85%, 90% or 95% sequence identity to SEQ ID NO: 41-43, 45-47, or 49-51;
(c) a polynucleotide that encodes a polypeptide having at least 85%, 90% or 95% sequence identity to SEQ ID NO:44, 48, or 52; and
(d) a polynucleotide that encodes a polypeptide of SEQ ID NO: 44, 48, or 52.

41. The wheat plant of claim 37, wherein the one or more endogenous wheat MS45 polynucleotide sequences is selected from the group consisting of:
(a) a polynucleotide comprising the sequence set forth in SEQ ID NO:61-63, 65-67, or 69-71;
(b) a polynucleotide having at least 85%, 90% or 95% sequence identity to SEQ ID NO: 61-63, 65-67, or 69-71;
(c) a polynucleotide that encodes a polypeptide having at least 85%, 90% or 95% sequence identity to SEQ ID NO:64, 68, or 72; and
(d) a polynucleotide that encodes a polypeptide of SEQ ID NO: 64, 68, or 72.

42. The wheat plant of claim 37, wherein the at least one altered target site comprises a genetic modification of one or more nucleotide substitutions, additions and/or deletions in or near one or more endogenous wheat male fertility MS9, MS22, MS26 or MS45 polynucleotides.

43. The wheat plant of claim 42, wherein the genetic modification is introduced by a Cas9 endonuclease guided by at least one guide RNA.

44. The wheat plant of claim 37, wherein the MS9, MS22, MS26, or MS45 polypeptide that confers male fertility to the male-sterile wheat plant is from a monocot.

45. The wheat plant of claim 37, wherein the MS9, MS22, MS26, or MS45 polypeptide that confers male fertility to the male-sterile wheat plant is from wheat or a wheat species.

46. The wheat plant of claim 37, wherein the MS9, MS22, MS26, or MS45 polypeptide that confers male fertility to the male-sterile wheat plant is from a species different than wheat.
